(19) United States
(12) Patent Application Publication
(10) Pub. No.: US 2012/0088737 A2
(43) Pub. Date: Apr. 12, 2012

(54) NOVEL ACYL GUANIDINE DERIVATIVES

(75) Inventors: Wataru MIYANAGA, Kawasaki-shi, Kanagawa (JP); Yoichiro SHIMA, Kawasaki-shi, Kanagawa (JP); Misato NOGUCHI, Kawasaki-shi, Kanagawa (JP); Akiko OONUKI, Kawasaki-shi, Kanagawa (JP); Yayoi KAWATO, Kawasaki-shi, Kanagawa (JP); Hiroshi IWATA, Kawasaki-shi, Kanagawa (JP); Eri HARADA, Kawasaki-shi, Kanagawa (JP); Ryuta TAKASHITA, Kawasaki-shi, Kanagawa (JP); Hirokazu UENO, Kawasaki-shi, Kanagawa (JP); Tadakiyo NAKAGAWA, Kawasaki-shi, Kanagawa (JP)

(73) Assignee: AJINOMOTO CO., INC, Tokyo (JP)

(21) Appl. No.: 12/896,387

(22) Filed: Oct. 1, 2010

(30) Foreign Application Priority Data
Oct. 2, 2009 (JP) 2009-230640
Jun. 4, 2010 (JP) 2010-129190

(51) Int. Cl. A61K 31/165; C07C 235/34; C07C 235/78; C07C 311/08; C07C 235/84; C07F 5/02; C07C 255/61; C07D 317/60; C07D 319/16; C07D 333/24; C07D 207/337; C07D 295/155; A61K 31/5375; A61K 31/24; A61K 31/18; A61K 31/195; A61K 31/69; A61K 31/277; A61K 31/36; A61K 31/357; A61K 31/381; A61K 31/40; A61P 13/12; A61P 43/00; A61P 9/12; A61P 25/20; C07C 233/40

(52) U.S. Cl. 514/64; 564/161; 564/171; 564/169; 560/34; 564/99; 564/156; 562/439; 564/8; 564/166; 558/414; 549/441; 549/366; 549/77; 548/561; 544/168; 514/238.5; 514/617; 514/622; 514/621; 514/539; 514/605; 514/616; 514/563; 514/619; 514/522; 514/466; 514/452; 514/438; 514/427

(57) ABSTRACT

The present invention provides a pharmaceutical which possesses an excellent inhibitory effect on NHE3 (Na⁺/H⁺ exchanger type 3) and effectively improves diseases or conditions of organs in which NHE3 is expressed.

### BACKGROUND OF THE INVENTION

The present invention relates to pharmaceuticals, particularly to novel acyl guanidine derivatives which possess inhibitory effects on Na⁺/H⁺ exchanger type 3 (hereinafter sometimes referred to as NHE3) and can be orally administered, methods of producing thereof, synthetic intermediates thereof and pharmaceutical compositions comprising the acyl guanidine derivatives.

Na⁺/H⁺ exchanger (NHE) is a transporter protein having 12-transmembrane domain which exists on the cell membrane and nine isoforms (NHE1/SLC9A1˜NHE8/SLC9A9) have been identified (Malo M E, Fliegel L. Can J. Physiol. Pharmacol. 2006; 84(11): 1081-95). In its C-terminal end domain which is located inside the cell, there are binding domains for variety of factors which are involved in intracellular signal regulations and it is believed that it plays a role in regulating cellular functions by interacting with such factors (Rhysiol. Review 2007, v87, pp 825-872). NHE is a very important protein, since it is involved in maintaining intracellular pH or moisture and regulating cell proliferation through exchange transport of H⁺ ions to the extracellular region by using concentration gradients, as driving force, which are generated between the intracellular region and the extracellular region.

NHE3 is highly-expressed in renal tubule and gastrointestinal tract and in particular, plays an important role in regulating Na concentration and pH in the body fluid (Bookstein C, DePaol A M, Xie Y, Niu P, Musch M W, Rao M C, Chang E B. J Clin Invest. 1994; 93(1): 106-13). It has been reported that proteinuria and loose stool occur in NHE3 knockout mice (Schultheis P J, Clarke L L, Meneton P, Miller M L, Soleimani M, Gawenis L R, Riddle T M, Duffy J J, Doetschman T, Wang T, Giebisch G, Aronson P S, Lorenz J N, Shull G E. Nat Genet. 1998; 19(3): 282-5) and thus its link to protein reabsorption and regulation of the amount of water in stool have been functionally demonstrated.

In recent years, involvements of NHE3 in pathological conditions such as diabetic nephropathy and metabolic syndrome-related nephropathy has been reported (Klisic J, Nief V, Reyes L, Ambuhl P M. Nephron Physiol. 2006; 102(2): 27-35). Glomerular hyperfiltration occurs in an early stage of these pathological conditions and results in edema or hypertension through enhancement of Na⁺ reabsorption. It is NHE3 that plays an important role in such an event. It has been reported that NHE3 expression is enhanced by albumin or glucose load in vitro (Stevens V A, Saad S, Poronnik P, Fenton-Lee C A, Polhill T S, Pollock C A. Nephrol Dial Transplant. 2008; 23(6): 1834-43) and thus it has been also believed that NHE3 expression is increased in kidney diseases such as diabetic nephropathy and hypertension, which leads to worsen early symptoms.

NHE inhibitors have long been developed and clinical trials have been conducted to validate whether such inhibitors can be used as pharmaceuticals. It has been demonstrated that Cariporide which is a selective inhibitor against NHE1 is effective for myocardial ischemic injury and it is assumed that Cariporide inhibits progression of myocardial damage by inhibiting H⁺ increase occurred during ischemic reperfusion and enhancement of Na⁺ excretion associated with the H⁺ increase. In addition, it has been reported that S3226 which has been reported to selectively inhibit NHE3 shows an ameliorating effect on renal ischemia and reperfusion injury (Hropot M, Juretschke H P, Langer K H, Schwark J R. Kidney Int. 2001; 60(6): 2283-2289).

As NHE3 inhibitors, derivatives having a diacylguanidine structure such as S3226 (EP0825178A and WO2001/87829), derivatives having an aminoimidazole structure (WO2005/26173), derivatives having a tetrahydroisoquinolin structure (WO2004/85404) and the like have been known.

Although derivatives having a structure represented by formula (I) wherein one of R⁶, R⁷, R⁸, R⁹ and R¹⁰ is a sulfonamide group have been reported as derivatives having a monoacylguanidine structure (WO2002/24637), no specific substitution position, no functional group other than sulfonamide group or no specificity is described. In addition, although derivatives having a structure represented by formula (I) wherein X is selected from the group consisting of a single bond, an oxygen atom and a sulfur atom have been reported as NHE inhibitors (Japanese Patent Unexmined Publication Hei 10-175939), no specific substitution position or no specific function group which is required for R⁶, R⁷, R⁸, R⁹ and R¹⁰ is described.

Any excellent NHE3 inhibitor or any NHE3 inhibitor targeted for diseases or conditions of organs in which NHE3 is expressed has not yet been obtained and thus there has been a demand for such a NHE3 inhibitor.

### DISCLOSURE OF INVENTION

An object of the present invention is to provide a pharmaceutical which possesses an inhibitory effect on NHE3 (Na⁺/H⁺ exchanger type 3) and effectively improves diseases or conditions of organs in which NHE3 is expressed.

Another object of the present invention is to provide a novel acylguanidine compound.

Yet another object of the present invention is to provide a novel acylguanidine compound which has good oral absorption.

Further, another object of the present invention is to provide a pharmaceutical composition.

The inventors of the present invention have intensively studied about compounds having inhibitory effects on NHE3 which are useful as pharmaceuticals improving diseases or conditions of organs in which NHE3 is expressed. As a result, compounds of formulae (I), (II) and (III) have been found to achieve the present invention.

Namely, the present invention provides a pharmaceutical comprising, as an active ingredient, an acylguanidine compound of formula (I), (II) or (III) or a pharmaceutically acceptable salt thereof, which effectively improves diseases or conditions of organs in which NHE3 is expressed.

The inventors of the present invention have intensively studied about compounds having inhibitory effects on Na⁺/H⁺ exchanger. As a result, it has been found that novel acylguanidine compounds have excellent inhibitory effects on Na⁺/H⁺ exchanger type 3 and thus the novel acylguanidine compounds are useful as pharmaceuticals which effectively improve diseases or conditions of organs in which NHE3 is expressed to achieve the present invention.

More specifically, the present invention provides an acylguanidine compound of the following formula (I) or a pharmaceutically acceptable salt thereof.

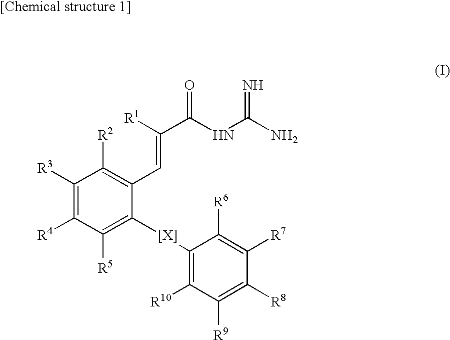

wherein

R¹ is a hydrogen atom, a halogen atom, a substituted or unsubstituted C₁₋₆-alkyl group;

R², R³, R⁴ and R⁵ are each independently selected from the group consisting of a hydrogen atom, a halogen atom, a hydroxy group, a substituted or unsubstituted C₁₋₆-alkyl group, a substituted or unsubstituted C₁₋₆-alkenyl group, a substituted or unsubstituted C₁₋₆-alkynyl group, a substituted or unsubstituted C₁₋₆-alkoxy group, a substituted or unsubstituted C₁₋₆-alkylthio group, a substituted or unsubstituted phenyloxy group and a substituted or unsubstituted phenyl group;

X is a single bond, —O— or —S—;

R⁶, R⁷, R⁸, R⁹ and R¹⁰ are each independently selected from the group consisting of a hydrogen atom, a halogen atom, a nitro group, a nitrile group, a carboxyl group, a hydroxy group, —B(OH)₂, a substituted or unsubstituted amidino group, a substituted or unsubstituted C₁₋₆-alkyl group, a substituted or unsubstituted C₁₋₆-alkenyl group, a substituted or unsubstituted C₁₋₆-alkynyl group, a substituted or unsubstituted C₁₋₆-alkoxy group, a substituted or unsubstituted C₁₋₆-alkylthio group, a substituted or unsubstituted aminocarbonyl group, a substituted or unsubstituted C₁₋₆-alkyl-carbonyl group, a substituted or unsubstituted C₁₋₆-alkoxy-carbonyl group, a substituted or unsubstituted C₁₋₆-alkyl-S(═O)₂—NH group and —OP, or

two adjacent groups from R⁶, R⁷, R⁸ and R⁹ together may form a 5-membered or 6-membered heterocyclic ring which has one or two oxygen atom(s) as a hetero atom(s) constituting the ring; and

P is selected from the group consisting of a substituted or unsubstituted C₁₋₆-acyl group, a substituted or unsubstituted C₁₋₆-alkoxycarbonyl group and a substituted or unsubstituted C₁₋₆-alkylaminocarbonyl group.

In one embodiment, the followings are preferred in formula (I).

R², R³, R⁴ and R⁵ are each independently selected from the group consisting of a hydrogen atom, a halogen atom, a hydroxy group, a substituted or unsubstituted C₁₋₆-alkyl group, a substituted or unsubstituted C₁₋₆-alkenyl group, a substituted or unsubstituted C₁₋₆-alynyl group, a substituted or unsubstituted C₁₋₆-alkoxy group, a substituted or unsubstituted C₁₋₆-alkylthio group and a substituted or unsubstituted phenyl group; and

R⁶, R⁷, R⁸, R⁹ and R¹⁰ are each independently selected from the group consisting of a hydrogen atom, a halogen atom, a nitro group, a nitrile group, a carboxyl group, a hydroxy group, —B(OH)₂, a substituted or unsubstituted C₁₋₆-alkyl group, a substituted or unsubstituted C₁₋₆-alkenyl group, a substituted or unsubstituted C₁₋₆-alkynyl group, a substituted or unsubstituted C₁₋₆-alkoxy group, a substituted or unsubstituted C₁₋₆-alkylthio group, an aminocarbonyl, a substituted or unsubstituted C₁₋₆-alkylcarbonyl group, a substituted or unsubstituted C₁₋₆-alkoxycarbonyl group and a substituted or unsubstituted C₁₋₆-alkyl-S(═O)₂—NH group, or

two adjacent groups from R⁶, R⁷, R⁸ and R⁹ together may form a 5-membered or 6-membered heterocyclic ring which has one or two oxygen atom(s) as a hetero atom(s) constituting the ring.

The present invention also provides a compound of the following formula (II) or a pharmaceutically acceptable salt thereof.

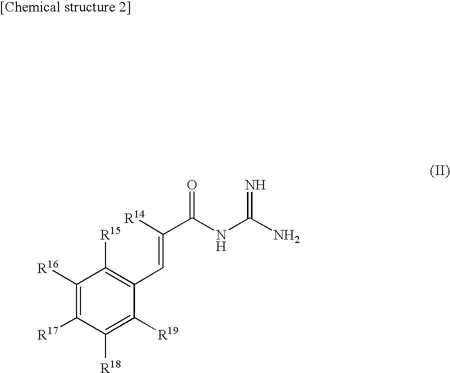

wherein

R¹⁴ is selected from the group consisting of a hydrogen atom, a halogen atom and a substituted or unsubstituted C₁₋₆-alkyl group;

R¹⁵ and R¹⁷ are each independently selected from the group consisting of a hydrogen atom, a halogen atom, a hydroxy group, a substituted or unsubstituted C₁₋₆-alkyl group, a substituted or unsubstituted C₁₋₆-alkoxy group, a substituted or unsubstituted phenyloxy group, a substituted or unsubstituted phenyl group and a substituted or unsubstituted, 5-membered or 6-membered heterocyclic ring having one or more hetero atom(s) selected from the group consisting of a nitrogen atom, an oxygen atom and a sulfur atom, the heterocyclic ring(s) being selected from the group consisting of a pyrrole ring, a furan ring, a thiophene ring, a thiazole ring, an isothiazole ring, an oxazole ring, an isoxazole ring, an imidazole ring, a pyrazole ring, a triazole ring, a tetrazole ring, a pyrimidine ring, a piperazine ring and a morpholine ring, provided that at least one of R¹⁵ and R¹⁷ is a heterocyclic ring; and

R¹⁶, R¹⁸ and R¹⁹ are each independently selected from the group consisting of a hydrogen atom, a halogen atom, a hydroxy group, a substituted or unsubstituted C₁₋₆-alkyl group, a substituted or unsubstituted C₁₋₆-alkoxy group, a substituted or unsubstituted phenyloxy group and a substituted or unsubstituted phenyl group.
The present invention further provides a compound of the following formula (III) or a pharmaceutically acceptable salt thereof.

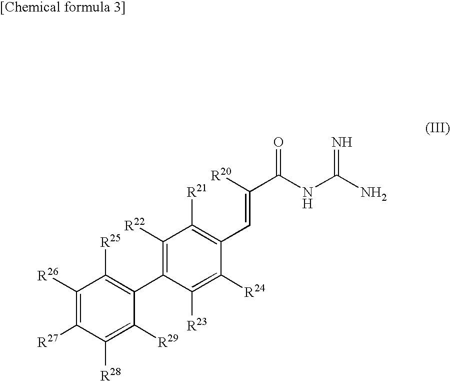

wherein

R²⁰ is selected from the group consisting of a hydrogen atom, a halogen atom and a substituted or unsubstituted C₁₋₆-alkyl group;

R²¹, R²², R²³ and R²⁴ are each independently selected from the group consisting of a hydrogen atom, a halogen atom, a hydroxy group, a substituted or unsubstituted C₁₋₆-alkyl group, a substituted or unsubstituted C₁₋₆-alkoxy group, a substituted or unsubstituted morpholine group and a substituted or unsubstituted piperazine group; and

R²⁵, R²⁶, R²⁷, R²⁸ and R²⁹ are each independently selected from the group consisting of a hydrogen atom, a halogen atom, a nitro group, a nitrile group, a carboxyl group, a hydroxy group, —B(OH)₂, a substituted or unsubstituted amidino group, a substituted or unsubstituted C₁₋₆-alkyl group, a substituted or unsubstituted C₁₋₆-alkenyl group, a substituted or unsubstituted C₁₋₆-alkynyl group, a substituted or unsubstituted C₁₋₆-alkoxy group, a substituted or unsubstituted aminocarbonyl group, a substituted or unsubstituted C₁₋₆-alkylcarbonyl group, a substituted or unsubstituted C₁₋₆-alkoxycarbonyl group and a substituted or unsubstituted C₁₋₆-alkyl-S(═O)₂—NH group, or

two adjacent groups from R²⁶, R²⁷, R²⁸ and R²⁹ together may form a 5-membered or 6-membered heterocyclic ring which has one or two oxygen atom(s) as a hetero atom(s) constituting the ring.

In one aspect, the present invention provides a pharmaceutical composition comprising a compound of formula (I), (II) or (III) or a pharmaceutically acceptable salt thereof and optionally a pharmaceutically acceptable carrier.

In another aspect, the present invention provides a pharmaceutical composition for treating or preventing a disease or condition of an organ in which NHE3 is expressed, which comprises a compound of formula (I), (II) or (III) or a pharmaceutically acceptable salt thereof and optionally a pharmaceutically acceptable carrier.

In a further aspect, the present invention provides a NHE3 inhibitor comprising a compound of formula (I), (II) or (III) or a pharmaceutically acceptable salt thereof and optionally a pharmaceutically acceptable carrier.

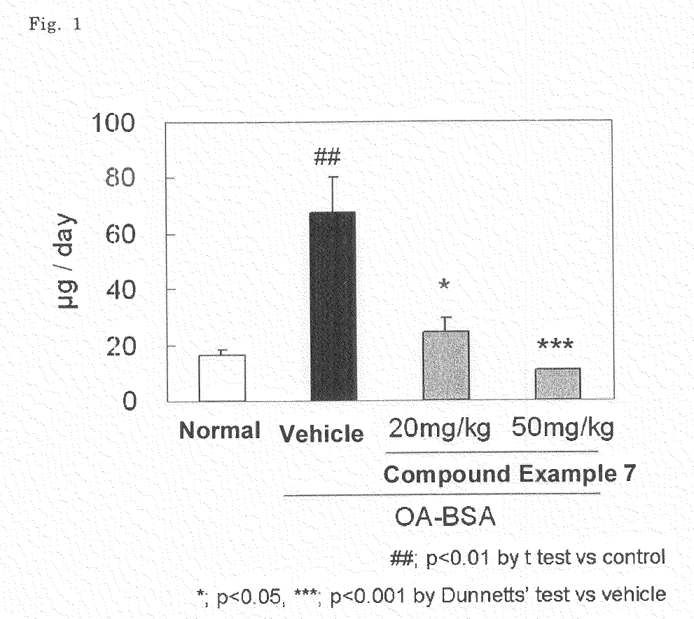

FIG. 1 shows a result for beta 2-microglobulin, which is a marker for tubular damage, after 4 days administration of the compound of Example 7.

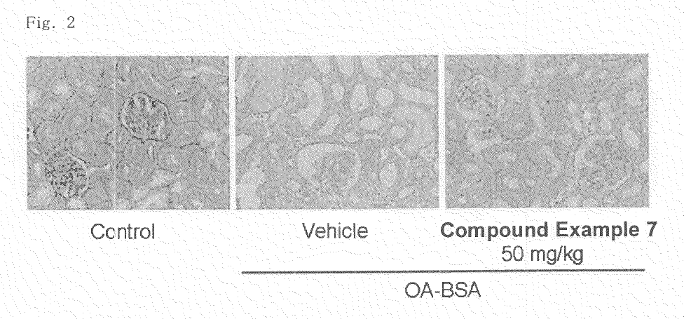

FIG. 2 shows a pathological tissue image (PAS stain) after 4 days administration of the compound of Example 7.

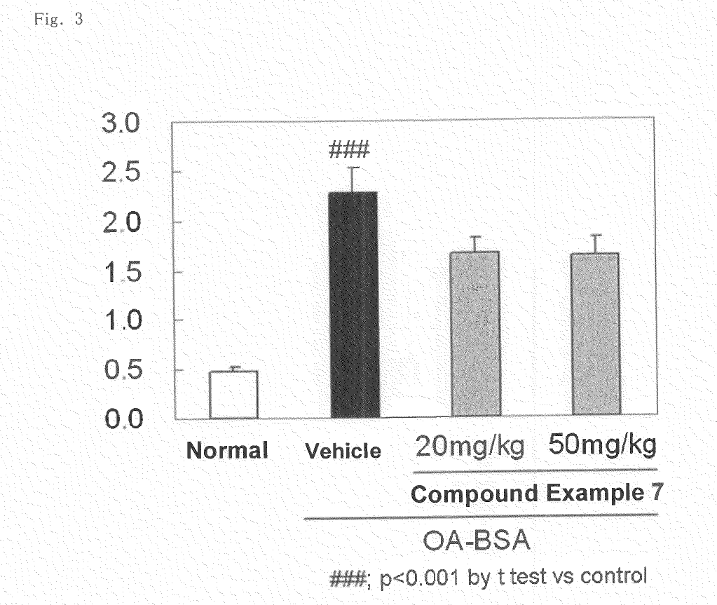

FIG. 3 shows a graph of tubular damage score after 4 days administration of the compound of Example 7.

Terms used therein are defined hereinafter.

Examples of a halogen atom include a fluorine atom, a chlorine atom, a bromine atom and an iodine atom.

“C₁₋₆-alkyl group” means a straight, branched, cyclic or partially-cyclic aliphatic hydrocarbon group having 1 to 6 carbon(s) and includes, for example, a methyl group, an ethyl group, a propyl group, an isopropyl group, a butyl group, an isobutyl group, a sec-butyl group, a tert-butyl group, a cyclopropylmethyl group, a cyclobutyl group, a pentyl group, an isopentyl group, a 1,1-dimethyl-propyl group, a cyclopropyl group, a cyclopentyl group, a hexyl group, a cyclohexyl group and the carbon number is preferably 1 to 3.

“C₁₋₆-alkenyl group” means a straight, branched or cyclic alkenyl group having 1 to 6 carbon(s) and specifically includes, for example, a 1-propenyl group, a 2-propenyl group, an isopropenyl group, a 1-butenyl group, a 2-butenyl group and a 3-butenyl group.

“C₁₋₆-alkynyl group means a straight or branched alkynyl group having 1 to 6 carbon(s) and specifically includes, for example, an ethynyl group, a 1-propynyl group, a 2-propynyl group, a 1-butynyl group, a 2-butynyl group and a 3-butynyl group.

“C₁₋₆-alkoxy group” means a straight, branched or cyclic alkoxy group having 1 to 6 carbon(s) and specifically includes, for example, a methoxy group, an ethoxy group, a n-propoxy group, a n-butoxy group, a n-pentyloxy group, a n-hexyloxy group, an isopropoxy group, an isobutoxy group, a sec-butoxy group, a tert-butoxy group, a cyclopropyloxy group, a cyclobutoxy group, a cyclopentyloxy group and a cyclohexyloxy group, and the carbon number is preferably 1 to 3.

“C₁₋₆-alkylthio group” means a straight, branched or cyclic alkylthio group and specifically includes, for example, a methylthio group, an ethylthio group, a n-propylthio group, a n-butylthio group, a n-pentylthio group, a n-hexylthio group, an isopropylthio group, an isobutylthio group, a sec-butylthio group and a tert-butylthio group, and the carbon number is preferably 1 to 3.

“A 5-membered or 6-membered heterocyclic ring which has one or two oxygen atom(s) as a hetero atom(s) constituting the ring” is preferably, but not specifically limited to, a 5-membered or 6-membered heterocyclic ring which has two oxygen atoms as hetero atoms constituting the ring and the rings represented by the following formulae are most preferable.

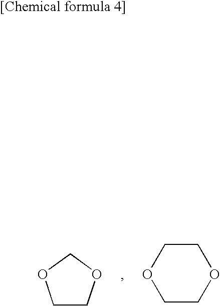

“Substituted” means that a group modified with the term has at least one substituent(s) selected from the following atoms or groups. Each substituent may be identical or different, and substitution position or substitution number may be any position or number and are not specifically limited.

Substituents are selected from the group consisting of halogen atoms, a hydroxy group, a mercapto group, a nitro group, a cycno group, an alkyl group, an alkoxy group, an alkylthio group, an alkylsulfonyl group, an acyl group, an acyloxy group, a carboxyl group, an alkoxycarbonyl group, a carbamoyl group, a sulfonamide group, an aryl group and a hetero aryl group.

“Substituted or unsubstituted” means that a group modified with the term may have no substituent or may have one or more substituent(s). Such substituents may be identical or different, and substitution position or substitution number may be any position or number and is not specifically limited. Such substituents preferably are selected from the group consisting of halogen atoms, a hydroxy group, a mercapto group, a nitro group, a cyano group, an alkyl group, an alkoxy group, an alkylthio group, an alkylsulfonyl group, an acyl group, an acyloxy group, a carboxyl group, an alkoxycarbonyl group, a carbamoyl group, a sulfonamide group, an aryl group and a heteroaryl group.

The compounds of the present invention are compounds of formulae (I), (II) and (III) and have an acryloyl group. Based on such structures, there are cis-trans geometrical isomers (or (E) isomer and (Z) isomer). The present invention encompasses an individual isomer ((E) isomer or (Z) isomer) or a mixture of the isomers. Among the compounds of the present invention, compounds having a trans configuration are particularly preferred. In addition, in the compounds of the present invention, there are tautomers based on their acylguanidine structure. The present invention encompasses an individual tautomer or a mixture of the tautomers. Other than the above-explained isomers or tautomers, there may be geometrical isomers or tautomers depending on kinds of substituents. The present invention encompasses an individual isomer or tautomer or a mixture of such isomers or tautomers. The compounds of the present invention may have an asymmetric carbon atom and in that case, there may be enantiomers (optical isomers) of (R) isomer and (S) isomer based on the asymmetric carbon atom. The present invention encompasses an individual enantiomer or a mixture of the enantiomers.

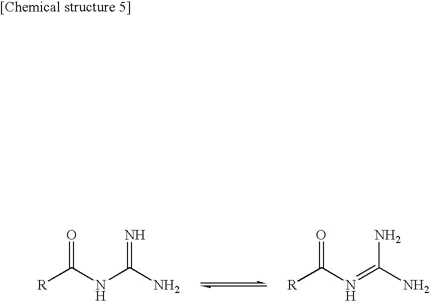

In the present invention, compounds having a combination of preferable groups for each substituent are preferable.

More specifically, the following groups for each substituent in formula (I) are preferable as an acylguanidine compound of formula (I) or a pharmaceutically acceptable salt thereof.

X is preferably a single bond or —O—, and more preferably a single bond.

R¹ is preferably selected from the group consisting of a hydrogen atom, a halogen atom, a methyl group and an ethyl group, and more preferably selected from a hydrogen atom or a methyl group.

R², R³, R⁴ and R⁵ are each independently preferably selected from the group consisting of a hydrogen atom, a halogen atom, a hydroxy group, a methyl group, an ethyl group, a methoxy group, an ethoxy group and a phenyl group substituted with a hydroxy group, more preferably selected from the group consisting of a hydrogen atom, a halogen atom, a hydroxy group, a methyl group and a methoxy group, and most preferably are selected from the group consisting of a hydrogen atom, a halogen atom and methyl group.

R⁶, R⁷, R⁸, R⁹ and R¹⁰ are each independently preferably selected from the group consisting of a hydrogen atom, a halogen atom, a nitro group, a nitrile group, a carboxyl group, a hydroxy group, —B(OH)₂, a substituted or unsubstituted C₁₋₆-alkyl group, a substituted or unsubstituted C₁₋₆-alkoxy-carbonyl group, a substituted or unsubstituted C₁₋₆-alkyl-S(═O)₂—NH group, a substituted or unsubstituted amidino group and a substituted or unsubstituted aminocarbonyl group, more preferably selected from the group consisting of a carboxyl group, a hydroxy group, —B(OH)₂, a 1-hydroxyethyl group, CH₃—S(═O)₂—NH group, an amidino group and HONHC(═O) group, and most preferably are a hydroxy group.

In addition to those preferable groups, R⁷, R⁸ and R⁹ are each independently preferably a hydroxy group, and R⁸ is most preferably a hydroxy group.

Compounds having a combination of the following groups for each substituent in formula (I) are preferable as an acylguanidine compound of formula (I) or a pharmaceutically acceptable salt thereof.

R⁵ is a hydrogen atom or a methyl group and R⁶ and R¹⁰ are each independently selected from the group consisting of a hydrogen atom, a halogen atom, a hydroxy group, a methoxy group and a substituted or unsubstituted C₁₋₆-alkyl group.

Further, R² is more preferably a hydrogen atom.

More preferably, R¹ is a hydrogen atom or a C₁₋₆-alkyl group.

Moreover, compounds having a combination of the following groups for each substituent in formula (I) are also preferable as an acylguanidine compound of formula (I) or a pharmaceutically acceptable salt thereof.

R³ is selected from the group consisting of a hydrogen atom, a hydroxy group, a substituted or unsubstituted C₁₋₆-alkyl group, a substituted or unsubstituted C₁₋₆-alkoxy group and a substituted or unsubstituted phenyl group and R⁴ is selected from the group consisting of a hydrogen atom, a fluorine atom, a hydroxy group, a substituted or unsubstituted C₁₋₆-alkyl group, a substituted or unsubstituted C₁₋₆-alkoxy group, a substituted or unsubstituted phenyloxy group and a substituted or unsubstituted phenyl group.

In addition, in the definitions for each substitutent, the substituted or unsubstituted phenyl group is preferably selected from the group consisting of a unsubstituted phenyl group and a hydroxy phenyl group, and the substituted or unsubstituted phenyloxy group is preferably selected from the group consisting of a unsubstituted phenyloxy group and a hydroxyphenyloxy group.

Furthermore, in the definitions for each substituent, each “C₁₋₆” is more preferably C₁₋₃.

The following groups for each substituent in formula (II) are preferable as an acylguanidine compound of formula (II) or a pharmaceutically acceptable salt thereof.

R¹⁴ is preferably selected from the group consisting of a hydrogen atom and a substituted or unsubstituted C₁₋₆-alkyl group.

R¹⁶ is preferably a hydrogen atom or a methyl group.

R¹⁵ and R¹⁷ are each independently preferably selected from a substituted or unsubstituted, 5-membered or 6-membered hetero ring which contains therein one or more hetero atom(s) selected from the group consisting of nitrogen, oxygen and sulfur, more preferably selected from the group consisting of a substituted or unsubstituted furan ring, a substituted or unsubstituted pyrrole ring, a substituted or unsubstituted thiophene ring, a substituted or unsubstituted pyrazole ring and a substituted or unsubstituted imidazole ring, and most preferably a pyrrole ring.

R¹⁹ is preferably selected from the group consisting of a hydrogen atom, a halogen atom and a methyl group.

If R¹⁵ is selected from the above-described hetero ring, R¹⁷ is preferably selected from the group consisting of a hydrogen atom, a fluorine atom, a hydroxy group, a substituted or unsubstituted C₁₋₆-alkyl group and a substituted or unsubstituted C₁₋₆-alkoxy group.

Furthermore, in the definitions for each substituent, each “C₁₋₆” is more preferably C₁₋₃.

In addition, compounds having a combination of the following groups for each substituent in formula (II) are preferable as an acylguanidine compound of formula (II) or a pharmaceutically acceptable salt thereof.

R¹⁴ is preferably selected from the group consisting of a hydrogen and a substituted or unsubstituted C₁₋₆-alkyl group, and more preferably selected from the group consisting of a hydrogen atom and a methyl group.

R¹⁶ is preferably a hydrogen atom or a methyl group.

R¹⁷ is preferably selected from the group consisting of a hydrogen atom, a fluorine atom, a hydroxy group, a substituted or unsubstituted C₁₋₆-alkyl group and a substituted or unsubstituted C₁₋₆-alkoxy group.

R¹⁵ is preferably selected from a substituted or unsubstituted, 5-membered or 6-membered hetero ring which contains therein one or more hetero atom(s) selected from the group consisting of nitrogen, oxygen and sulfur, more preferably selected from the group consisting of a substituted or unsubstituted furan ring, a substituted or unsubstituted pyrrole ring, a substituted or unsubstituted thiophene ring, a substituted or unsubstituted pyrazole ring and a substituted or unsubstituted imidazole ring, and most preferably a pyrrole ring.

Furthermore, in the definitions for each substituent, each “C₁₋₆” is more preferably C₁₋₃.

Moreover, compounds having a combination of the following groups for each substituent in formula (II) are also preferable as an acylguanidine compound of formula (II) or a pharmaceutically acceptable salt thereof.

R¹⁴ is preferably selected from the group consisting of a hydrogen atom and a substituted or unsubstituted C₁₋₆-alkyl group, and more preferably selected from the group consisting of a hydrogen atom and a methyl group.

R¹⁵ and R¹⁹ are each independently preferably selected from the group consisting of a hydrogen atom and a methyl group.

R¹⁷ is preferably selected from a substituted or unsubstituted, 5-membered or 6-membered hetero ring which contains therein one or more hetero atom(s) selected from the group consisting of nitrogen, oxygen and sulfur, and more preferably selected from the group consisting of a substituted or unsubstituted furan ring, a substituted or unsubstituted pyrrole ring, a substituted or unsubstituted thiophene ring, a substituted or unsubstituted pyrazole ring and a substituted or unsubstituted imidazole ring.

Furthermore, in the definitions for each substituent, each “C₁₋₆” is more preferably C₁₋₃.

The following groups for each substituent in formula (III) are preferable as an acylguanidine compound of formula (III) or a pharmaceutically acceptable salt thereof.

R²¹ and R²⁴ are each independently preferably selected from the group consisting of a hydrogen atom, a halogen atom, a hydroxy group, a substituted or unsubstituted C₁₋₆-alkyl group, a substituted or unsubstituted C₁₋₆-alkoxy group, a substituted or unsubstituted morpholine group and a substituted or unsubstituted piperazine group.

R²² and R²³ are each independently preferably selected from the group consisting of a hydrogen atom, a halogen atom, a hydroxy group and a substituted or unsubstituted C₁₋₆-alkyl group, and more preferably selected from the group consisting of a hydrogen atom, a halogen atom, a methyl group and an ethyl group.

R²⁵ and R²⁹ are each independently preferably selected from the group consisting of a hydrogen atom, a halogen atom, a hydroxy group and a substituted or unsubstituted C₁₋₆-alkyl group, and more preferably selected from the group consisting of a hydrogen atom, a halogen atom, a methyl group and an ethyl group.

One of R²⁵, R²⁶, R²⁷, R²⁸ and R²⁹ is preferably a hydroxy group, and more preferably, one of R²⁶, R²⁷ and R²⁸ is a hydroxy group.

Moreover, compounds having a combination of the following groups for each substituent in formula (III) are also preferable as an acylguanidine compound of formula (III) or a pharmaceutically acceptable salt thereof.

R²¹ and R²⁴ are each independently preferably selected from the group consisting of a hydrogen atom, a halogen atom, a hydroxy group, a substituted or unsubstituted C₁₋₆-alkyl group, a substituted or unsubstituted C₁₋₆-alkoxy group, a substituted or unsubstituted morpholine group and a substituted or unsubstituted piperazine group, more preferably selected from the group consisting of a hydrogen atom, a halogen atom, a hydroxy group, a methyl group, an ethyl group, a methoxy group, an ethoxy group and morpholine group, and most preferably selected from the group consisting of a hydrogen atom, a methyl group and a morpholine group.

R²² and R²³ are each independently preferably selected from the group consisting of a hydrogen atom and a substituted or unsubstituted C₁₋₆-alkyl group, and more preferably selected from the group consisting of a hydrogen atom and a methyl group.

R²⁵ and R²⁹ are each independently preferably selected from the group consisting of a hydrogen atom, a halogen atom, a hydroxy group and a substituted or unsubstituted C₁₋₆-alkyl group, and more preferably selected from the group consisting of a hydrogen atom, a halogen atom, a hydroxy group, a methoxy group, a methyl group and an ethyl group.

One of R²⁶, R²⁷ and R²⁸ is preferably selected from the group consisting of a hydroxymethyl group and a hydroxy group, and more preferably, R²⁷ is a hydroxy group.

Furthermore, in the definitions for each substituent, each “C₁₋₆” is more preferably C₁₋₃.

Representative methods for manufacturing the present compounds of formulae (I), (II) and (III) will be explained hereinafter. Most of the compounds of the present invention can be synthesized, for example, by using the following manufacturing methods.

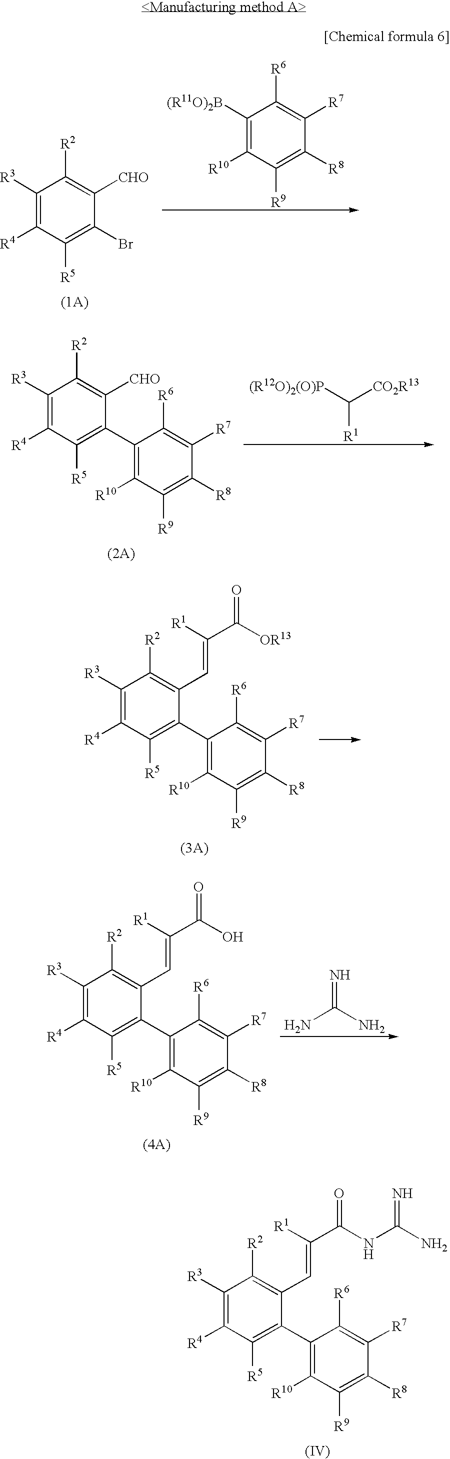

In the above described Manufacturing method A, R¹, R², R³, R⁴, R⁵, R⁶, R⁷, R⁸, R⁹ and R¹⁰ are those defined hereinbefore. R¹¹, R¹² and R¹³ are each independently selected from the group consisting of a hydrogen atom, a C₁₋₅-alkyl group which may be substituted with a halogen atom and benzyl group which may be substituted with a halogen atom. In addition, two R¹¹ substitutents may form a ring by sharing a substituent or binding together.

Corresponding aldehyde (2A) can be synthesized by coupling corresponding bromoaldehyde (1A) with a corresponding phenylboronicacid derivative. Corresponding acrylic acid ester (3A) can be synthesized by reacting the resultant aldehyde (2A) with a corresponding phosphoryl derivative which has been treated under low temperature and under basic condition such as NaH, lithium diisopropylamide (LDA) and n-BuLi. Corresponding acrylic acid (4A) can be synthesized by hydrolyzing the resultant acrylic acid ester (3A) such as under an alkaline condition. Acylguanidine (IV) of the present invention can be synthesized by activating the resultant acrylic acid (4A) via an addition of a condensation agent such as 1,1′-carbonylbis-1H-imidazole (CDI) thereto and then adding 1M solution of guanidine-dimethylformaldehyde (DMF).

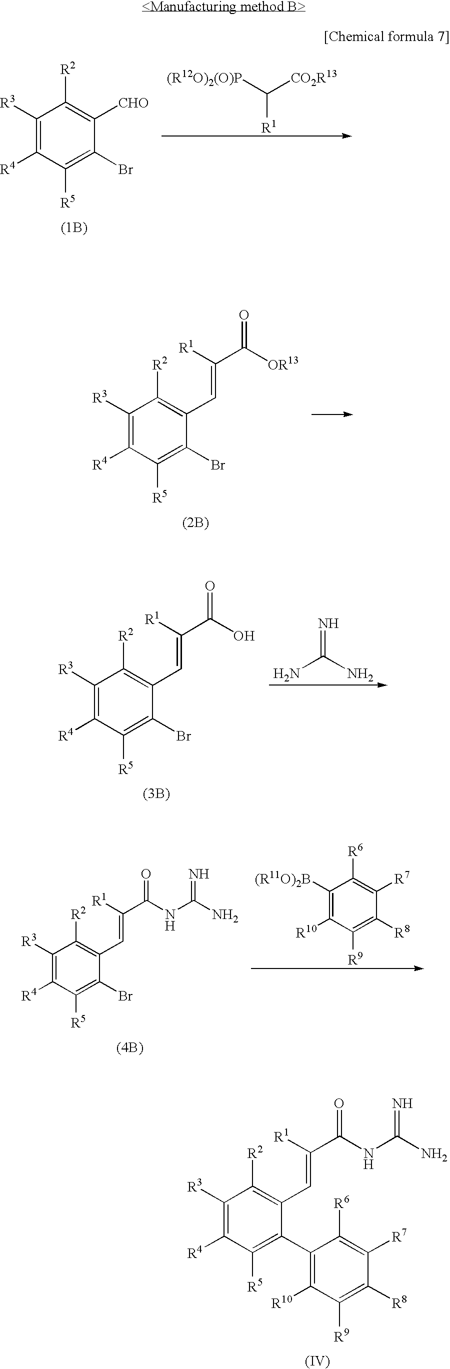

In the above described Manufacturing method B, R¹, R², R³, R⁴, R⁵, R⁶, R⁷, R⁵, R⁹ and R¹⁰ are those defined hereinbefore. R¹¹, R¹² and R¹³ are each independently selected from the group consisting of a hydrogen atom, a C₁₋₅-alkyl group which may be substituted with a halogen atom and benzyl group which may be substituted with a halogen atom. In addition, two R¹¹ substitutents may form a ring by sharing a substituent or binding together.

Corresponding acrylic acid ester (2B) can be synthesized by reacting corresponding 2-bromoaldehyde (1B) with a corresponding phosphoryl derivative which has been treated under low temperature and under basic condition such as NaH, lithium diisopropylamide (LDA) and n-BuLi. Corresponding acrylic acid (3B) can be synthesized by hydrolyzing the resultant acrylic acid ester (2B) such as under an alkaline condition. Acylguanidine (4B) can be synthesized by activating the resultant acrylic acid (3B) via an addition of a condensation agent such as 1,1′-carbonylbis-1H-imidazole (CDI) thereto and then adding 1M solution of guanidine-dimethylformaldehyde (DMF). Acylguanidine (IV) of the present invention can be synthesized by coupling the resultant acylguanidine (4B) with a corresponding phenylboronicacid derivative.

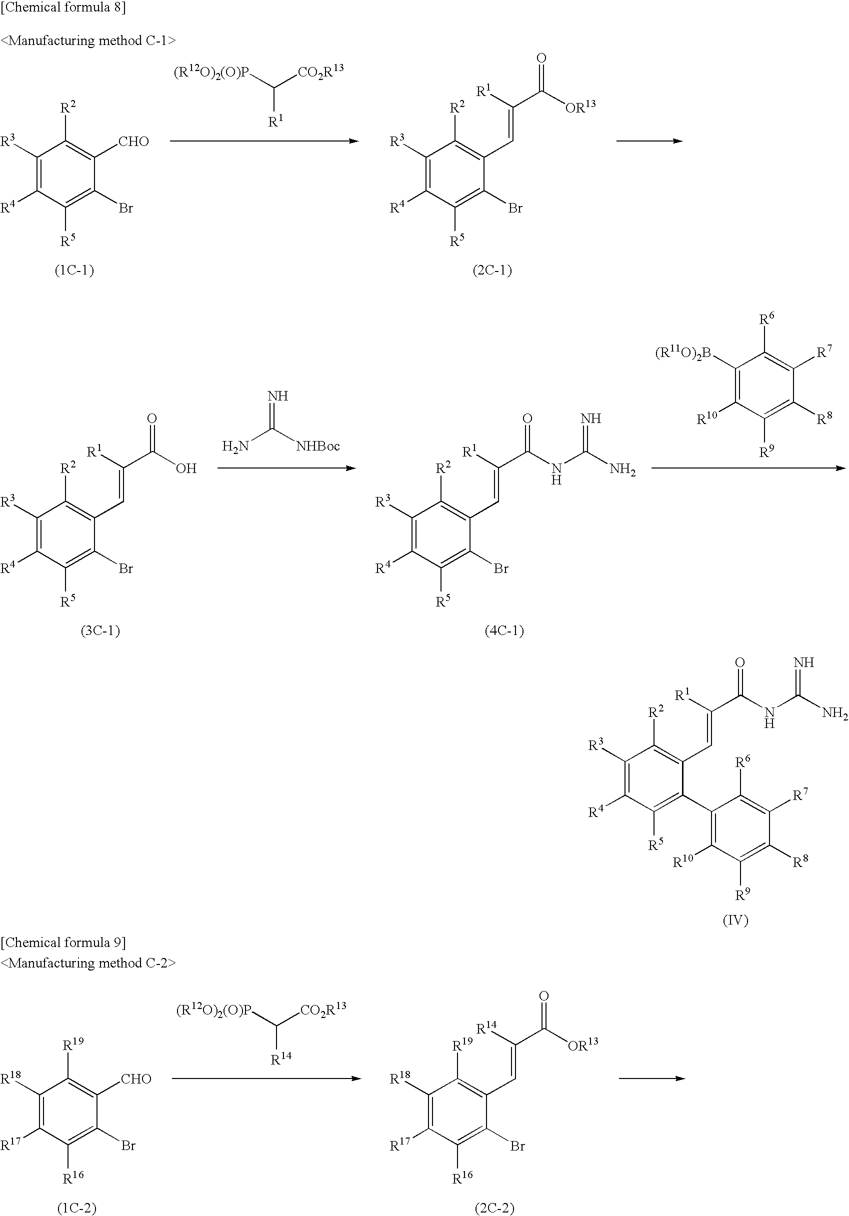

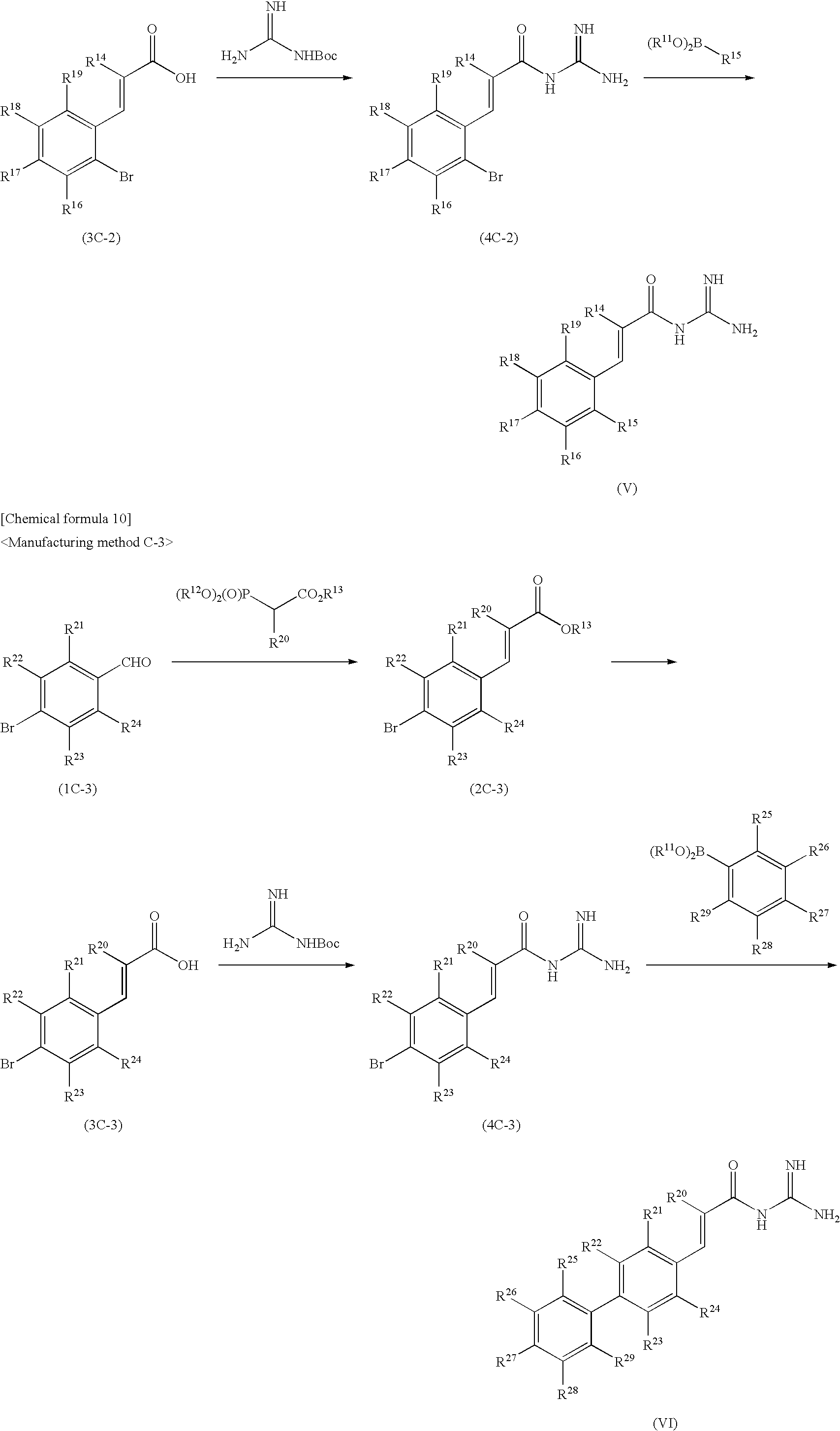

In the above described Manufacturing method C(C-1, C-2 and C-3), R¹, R², R³, R⁴, R⁵, R⁶, R⁷, R⁸, R⁹, R¹⁰, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸ and R²⁹ are those defined hereinbefore. R¹¹, R¹² and R¹³ are each independently selected from the group consisting of a hydrogen atom, a C₁₋₅-alkyl group which may be substituted with a halogen atom and benzyl group which may be substituted with a halogen atom. In addition, two R¹¹ substitutents may form a ring by sharing a substituent or binding together.

Corresponding acrylic acid ester (2C) can be synthesized by reacting corresponding 2-bromoaldehyde (1C) with a corresponding phosphoryl derivative which has been treated under low temperature and under basic condition such as NaH, lithium diisopropylamide (LDA) and n-BuLi. Corresponding acrylic acid (3C) can be synthesized by hydrolyzing the resultant acrylic acid ester (2C) such as under an alkaline condition. Acylguanidine (4C) can be synthesized by activating the resultant acrylic acid (3C) via an addition of a condensation agent such as 1,1′-carbonylbis-1H-imidazole (CDI) thereto and then conducting a condensation reaction with a guanidine protected with a tert-butoxycarbonyl (Boc) group. Acylguanidines (IV), (V) and (VI) of the present invention can be synthesized by coupling the resultant acylguanidine (4C) with a corresponding phenylboronicacid derivative.

The condensation of an acrylic acid with a guanidine derivative in the above described manufacturing methods A, B and C can be carried out by using any conventional method in the art and examples of such a conventional method include use of an acid halide, an acid anhydride, an active ester, a lower alkylester, an acid azide, an condensation agent.

Examples of such an acid halide include acid chlorides and acid bromides.

A symmetric acid anhydride or a mixed acid anhydride may be used as an acid anhydride and examples of such a mixed acid anhydride include a mixed acid anhydride with an alkyl chlorocarbonate ester such as ethyl chlorocarbonate and isobutyl chlorocarbonate, a mixed acid anhydride with an aralkyl chlorocarbonate ester such as benzyl chlorocarbonate, a mixed acid anhydride with an aryl chlorocarbonate ester such as phenyl chlorocarbonate and a mixed acid anhydride with an alkane acid such as isovaleric acid and pivalic acid.

Examples of such an active ester include p-nitrophenyl ester, N-hydroxysuccinimide ester, pentafluorophenyl ester, 2,4,5-trichlorophenyl ester, pentachlorophenyl ester, cyanomethyl ester, N-hydroxysuccinimide ester, N-hydroxyphthalimide ester, N-hydroxy-5-norbornene-2,3-dicarboxyimide ester, N-hydroxypiperidine ester, 8-hydroxyquinoline ester, 2-hydroxyphenyl ester, 2-hydroxy-4,5-dichlorophenyl ester, 2-hydroxypepiridine ester, 2-pyridylthiol ester and 1-benzotriazole.

Examples of such a condensation agent include, for example, dicyclohexylcarbodiimide (DCC), diisopropylcarbodiimide (DIPC), 1-ethyl-3-(3-dimethylaminopropyl) carbodiimide (WSC), benzotriazole-1-yl-tris(dimethylamino) phosphonium.hexafluorophosphate (BOP), diphenylphosphonylazide (DPPA), 1,1′-carbonyl bis-1H-imidazole (CDI) and the like.

If desired, an additive such as N-hydroxysuccinimide (HONSu), 1-hydroxybenzotriazole (HOBt), 3-hydroxy-4-oxo-3,4-dihydro-1,2,3-benzotriazine (HOOBt) may be further added.

In each step, any reaction conditions generally used in the art can be used and should be appropriately selected depending on kinds of staring compounds.

In addition, examples of a solvent used include, for example, an aromatic hydrocarbon solvent such as benzene, toluene and xylene, an ether solvent such as tetrahydrofuran and 1,4-dioxane, a halogenated hydrocarbon solvent such as dichloromethane, chloroform and 1,2-dichloroethane, an amide solvent such as dimethylformamide and dimethylacetamide, and a basic solvent such as pyridine. Each solvent may be used by itself or in combination with one or more other solvent(s) including water. Solvent(s) should be appropriately selected depending on kinds of starting compounds.

Manufacturing method C may be preferably used to carry out the present invention and CDI may be preferably used as a condensation agent.

The compounds of the present invention obtained by the above-explained methods can be purified by any conventional means generally used in the organic synthesis field such as extraction, distillation, crystallization, column chromatography and the like.

In the case where compounds of formulae (I), (II) and (III) according to the present invention may form a salt, such a salt may be any kind of salt as long as it is pharmaceutically acceptable. If there is an acidic group in a compound such as carboxyl group, examples of such a salt include, for example, an ammonium salt, a salt with an alkali metal such as sodium and potassium, a salt with an alkaline earth metal such as calcium and magnesium, an aluminum salt, a zinc salt, a salt with an organic amine such as triethylamine, ethanolamine, morpholine, piperizine and dicyclohexylamine, and a salt with a basic amine such as arginine and lysine for such an acidic group.

If there is a basic group in a compound, examples of such a salt include, for example, a salt with an inorganic acid such as hydrochloric acid, sulfuric acid, phosphoric acid, nitric acid, and hydrobromic acid, a salt with an organic carboxylic acid such as acetic acid, trifluoroacetic acid, citric acid, benzoic acid, maleic acid, fumaric acid, tartaric acid, succinic acid, tannic acid, butyric acid, hibenzoic acid, pamoic acid, enanthic acid, decanoic acid, teoclate, salicyclic acid, lactic acid, oxalic acid, mandelic acid and malic acid, and a salt with an organic sulfonic acid such as methanesulfonic acid, benzenesulfonic acid and p-toluenesulfonic acid.

Examples of a method of forming a salt include, for example, mixing a compound of formula (I), (II) or (III) with an appropriate acid or base at a suitable ratio in a solvent or dispersion, or conducting cation exchange or anion exchange from another salt form.

The compounds of the present invention encompass solvates of a compound of formula (I), (II) or (III), for example, hydrates, alcohol adducts and the like.

The compounds of the present invention may be converted to corresponding prodrug forms. The term “prodrug” used herein means a compound which will be converted (metabolized) in the body into the compound of the present invention. For instance, in the case where an active form has a carboxyl group or phosphate group, examples of a prodrug include their esters, amides and the like. In the case where an active form has an amino group, examples of a prodrug include its amides, carbamate and the like. In the case where an active form has a hydroxy group, examples of a prod-rug include its esters, carbonates, carbamates and the like. The compounds of the present invention may be converted into corresponding prodrugs by combining them with amino acid(s) or sugar group(s).

The acylguanidine derivatives of the present invention of formulae (I), (II) and (III) or a pharmaceutically acceptable salt thereof may be produced as a pharmaceutical composition with or without using a drug formulation auxiliary agent according to conventional means and then administered. Examples of a dosage form for the pharmaceutical composition include, for example, tablet, powder, injection solution, freeze-dried form for injection, pill, granunle, capsule, suppository, liquid, sugar coated tablet, depot, syrup, suspension, emulsion, troche, sublingual tablet, patch, orally-disintegrating tablet, inhalant, enema, ointment, tape, eye drop and the like.

The pharmaceutical composition or NHE3 inhibitor of the present invention may comprise any one of or any combination of two, three or more of the acylguanidine compounds of formulae (I), (II) and (III) or a pharmaceutically acceptable salt thereof and may further comprise any pharmaceutically, physiologically or experimentally acceptable, solid or liquid carriers, additives and the like.

Examples of such a carrier includes, for example, glucose, lactose, sucrose, starch, mannitol, dextrin, fatty acid glycerides, polyethylene glycol, hydroxyethylated starch, ethylene glycol, polyoxyethylene sorbitan fatty acid esters, gelatin, albumin, amino acids, water and saline. Moreover, any conventional additives such as stabilizing agents, wetting agents (humectants), emulsifying agents, binders, tonicity agents and the like may be appropriately added to the pharmaceutical composition or NHE3 inhibitor of the present invention, if necessary.

Examples of such an additive include, but not specifically limited to as long as they are generally used in the art for any purpose, for example, flavors, saccharides, sweeteners, dietary fibers, vitamins, amino acids such as monosodium glutaminate (MSG), nucleic acids such as inosine monophosphate (IMP), mineral salts such as sodium chloride, water and the like.

In addition, the pharmaceutical composition or NHE3 inhibitor may be used in any form such as dry powder, paste, solution and the like.

The pharmaceutical composition or NHE3 inhibitor of the present invention may be applied via any invasive or noninvasive administration method. Examples of such a method include, but not specifically limited to, oral administration, injection and the like. Administration of suppository or transdermal administration may be also employed.

An active ingredient may be formulated in any conventional pharmaceutical formulation together with any solid or liquid pharmaceutical carrier suitable for oral administration or injection and then administered. Examples of such a formulation include, for example, a solid formulation such as tablet, granule, powder and capsule, a liquid formulation such as solution, suspension and emulsion, and freeze dried formulation. These formulations can be prepared by any conventional means in the art. In addition, any pharmaceutically or experimentally acceptable, solid or liquid carriers, additives and the like may be optionally added to the pharmaceutical composition or NHE3 inhibitor of the present invention.

Although an amount of the pharmaceutical composition or NHE3 inhibitor of the present invention may be appropriately determined depending on each purpose, for instance, if it is orally administered to the subject, as the total amount of the acylguanidine compounds of formulae (I), (II) and (III) or a pharmaceutically acceptable salt thereof, it is preferably 0.0001 mg/kg˜5 g/kg of body weight per dose, more preferably 0.001 mg/kg˜1 g/kg of body weight per dose, and yet more preferably 0.01 mg/kg˜10 mg/kg of body weight per dose. Number of administration times is not specifically limited and it may be administered 1 time or plural times/day.

Although a content of the acylguanidine compounds of formulae (I), (II) and (III) or a pharmaceutically acceptable salt thereof in the pharmaceutical composition or NHE3 inhibitor is not specifically limited as long as it complies with the above-described amount to be used, it is preferably 0.000001 mass %˜99.9999 mass % based on the dry weight of the pharmaceutical composition or NHE3 inhibitor, more preferably 0.00001 mass %˜99.999 mass %, and particularly preferably 0.0001 mass %˜99.99 mass %.

The pharmaceutical composition or NHE3 inhibitor of the present invention may further comprise one or two or more kind(s) of known substance(s) which can produce clinically desired effect(s).

The pharmaceutical composition or NHE3 inhibitor can be used for any disease or condition for which it may produce clinically desired therapeutic or preventive effect(s) including NHE3-related diseases or conditions. Examples of such a disease or condition include, but not limited to, renal dysfunction, diabetic nephropathy, metabolic syndrome-related nephropathy, edema, hypertension, sleep apnea syndrome, renal ischemia, reperfusion injury and tubular damage, and tubular damage or renal dysfunction is preferred.

The acylguanidine compounds of formulae (I), (II) and (III) or a pharmaceutically acceptable salt thereof according to the present invention show good inhibitory effects on Na⁺/H⁺ exchanger type 3. Particularly preferred compounds among the compounds according to the present invention have good oral absorption. In addition, particularly preferred compounds among the compounds according to the present invention have good selectivities for NHE3.

The present invention will be explained in detail hereinafter by referring to the examples, which are not intended to be limiting of the present invention.

### EXAMPLES

### Listings of Abbreviations

AIBN: azoisobutyronitrile

Boc: tert-butoxycarbonyl

CDI: 1,1′-carbonylbis-1H-imidazole

DMA: dimethylacetamide

DMF: dimethylformamide

dppf: 1,1′-bis(diphenylphosphino)ferrocene

EtOAc: ethylacetate

EDCI: 1-(3-Dimethylaminopropyl)-3-ethylcarbodiimide Hydrochloride

HATU: O-(benzotriazol-1-yl)-N,N,N′,N′-tetramethyluroniumhexafluorophosphate

HPLC: high performance liquid chromatograph

MeOH: methanol

MS: analytical value by mass spectrometry (EI) [M+H]⁺

TFA: trifluoroacetic acid

THF: tetrahydrofuran

### Intermediate 1

### Synthesis of N-[(E)-3-(2-bromo-phenyl)-2-methyl-acryloyl]-guanidine (Manufacturing Method C)

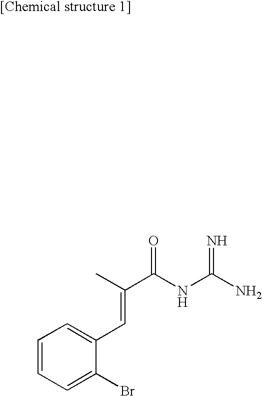 <Step 1>

NaH (60% assay, 824 mg, 20.6 mmol) was suspended in DMF (50 mL) and then cooled to 0° C. Triethyl 2-phosphonopropionate (4.48 mL, 20.6 mmol) in DMF (10 mL) was added dropwise in a slow manner to the resulting solution and stirred for 15 minutes. Then, 2-bromobenzaldehyde (2.0 mL, 17.0 mmol) in DMF (3 mL) was added thereto in a slow manner and stirred for 18 hours while gradually heating it from 0° C. to room temperature. EtOAc was added to the reaction solution, washed with water and saturated saline and then dried over anhydrous MgSO₄. The solvent was eliminated in vacuo to obtain a residue.

The resulting residue was dissolved in THF (50 mL) and MeOH (20 mL), 1 N NaOH (40 ml, 40 mmol) was added thereto and stirred at room temperature for 8 hours. The solvent was eliminated in vacuo, 2N HCl was added to acidify the solution and then the precipitated crystals were filtrated to obtain white crystals of the objective carboxylic acid (3.37 g, 82.0%).

MS: 241

<Step 2>

The carboxylic acid obtained from Step 1 (3.81 g, 15.7 mmol) was dissolved in DMF (40 mL), CDI (2.80 g, 17.3 mmol) was added thereto and then stirred at room temperature for 30 minutes. N-Boc-guanidine (3.75 g, 23.6 mmol) was added to the solution and then stirred for 16 hours. After eliminating the solvent in vacuo, TFA (10 mL) was added to the residue at 0° C. and then stirred for 1.5 hours. After concentrating the solvent in vacuo, it was purified by reversed phase HPLC (0.1% TFA in water/CH₃CN) to obtain the objective Intermediate 1 (3.48 g, 57.0%).

MS: 282

### Intermediate 2

### Synthesis of N-[(E)-3-(2-bromo-4-methyl-phenyl)-2-methyl-acryloyl]-guanidine

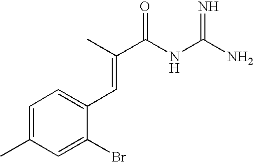 <Step 1>

NaH (60% assay, 502 mg, 12.6 mmol) was suspended in DMF (50 mL), triethyl-phosphonopropionate (2.74 mL, 12.6 mmol) was added dropwise in a slow manner to the resulting solution and then stirred for 15 minutes. Then, 2-bromo-4-methylbenzaldehyde (2 g, 10.1 mmol) in DMF (10 mL) was added thereto in a slow manner and stirred for 18 hours. EtOAc was added to the reaction solution, washed with water and saturated saline and then dried over anhydrous MgSO₄. After eliminating the solvent in vacuo, the resulting compound was dissolved in THF (15 mL) and MeOH (12 mL), 1N NaOH (8 mL, 8 mmol) was added and then stirred at room temperature for 8 hours. The solvent was eliminated in vacuo, 2N HCl was added to acidify the solution and then the precipitated crystals were filtrated to obtain the objective carboxylic acid (1.36 g, 42%).

MS: 241

<Step 2>

The carboxylic acid obtained from Step 1 (1.36 g, 5.3 mmol) was dissolved in DMF (10 mL), CDI (1.0 g, 6.4 mmol) was added and then stirred at room temperature for 30 minutes. N-Boc-guanidine (1.27 g, 8.0 mmol) was added to the solution and stirred for 16 hours. EtOAc was added, washed with water and saturated saline and then dried over anhydrous MgSO₄. TFA (10 mL) was added to the residue and then stirred for 1.5 hours. After concentrating the solvent in vacuo, it was purified by reversed phase HPLC (0.1% TFA in water/CH₃CN) to obtain the objective Intermediate 2 (820 mg, 38%).

MS: 297

### Intermediate 3

### Synthesis of N-[(E)-3-(2,6-dibromo-phenyl)-2-methyl-acryloyl]-guanidine

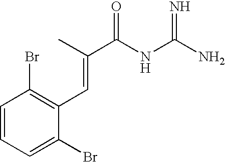 <Step 1>

Intermediate 3 was obtained in the same manner as described for Intermediate 1.

MS: 362

### Intermediate 4

### Synthesis of N-[(E)-3-(2,4-dibromo-phenyl)-2-methyl-acryloyl]-guanidine

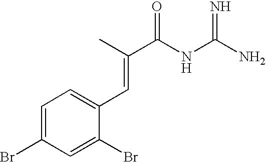 <Step 1>

AIBN (657 mg, 4.0 mmol) was added at room temperature into a container containing 2,4-dibromotoluene (5.00 g, 20 mmol), N-bromosuccinimide (3.92 g, 22.0 mmol) and carbon tetrachloride (6.0 mL). After stirring it at 65° C. for 16 hours, the solvent was eliminated in vacuo. The residue was filtrated, washed with hexane and then the filtrate was eliminated in vacuo to obtain a crude product (5.12 g, 78%).

<Step 2>

Tri-methylamine-N-oxide (1.17 g, 15.6 mmol) was added to the crude product obtained from Step 1 (5.12 g, 15.6 mmol) in acetonitrile (30 mL) and then stirred at 60° C. for 6 hours. After cooling it to room temperature, the solvent was eliminated in vacuo and then purified by silica gel column chromatography (Hexane/EtOAc) to obtain the objective aldehyde (2.49 g, 60%).

MS: 265

<Step 3>

NaH (60% assay, 1.13 g, 28.3 mmol) was suspended in THF (40 mL) and then cooled to 0° C. Tri-ethyl-2-phosphonopropionate (6.74 g, 28.3 mmol) in THF (5 mL) was added in a slow manner to the resulting suspension. After stirring it for 15 minutes, the aldehyde obtained from Step 2 (2.49 g, 9.435 mmol) in THF (5 mL) was added thereto and then stirred for 1 hour while gradually raising the temperature to room temperature. EtOAc was added thereto and then washed with NaHCO₃ solution, water and saturated saline. After drying it over anhydrous MgSO₄, the solvent was eliminated in vacuo to obtain a crude product (an ester intermediate).

MS: 349

The resulting crude product was dissolved in a mixed solution of THF/MeOH (v/v=5/3, 40 mL). Then, 2N NaOH (30 mL, 60 mmol) was added to the solution and stirred at 50° C. for 6 hours. After cooling it to 0° C., 2N HCl was added to acidify the solution, dichloromethane was added thereto, washed with water and saturated saline and then dried over anhydrous MgSO₄. After the solvent was eliminated in vacuo, it was purified by silica gel column chromatography (Hexane/EtOAc) to obtain the objective carboxylic acid (2.04 g, 68%).

MS: 321

<Step 4>

The carboxylic acid obtained from Step 3 (2.04 g, 6.375 mmol) was dissolved in DMF (20 mL), CDI (1.24 g, 7.65 mmol) was added thereto and then stirred at room temperature for 30 minutes. N-Boc-guanidine (1.22 g, 7.65 mmol) was added to the solution and then stirred for 19 hours. Then, the solvent was eliminated in vacuo, TFA (20 mL) was added to the residue and stirred at 55° C. for 8 hours. Then, the solvent was eliminated in vacuo, it was purified by reversed phase HPLC (0.1% TFA in water/CH₃CN) to obtain the objective Intermediate 4 (0.821 g, 27%).

MS: 362

### Intermediate 5

### Synthesis of N-[(E)-3-(4-bromo-phenyl)-2-methyl-acryloyl]-guanidine

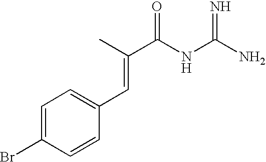 <Step 1>

NaH (60% assay, 412 mg, 10.3 mmol) was suspended in DMF (50 mL) and then cooled to 0° C. Tri-ethyl-2-phosphonopropionate (2.24 mL, 10.3 mmol) in THF (10 mL) was added dropwise in a slow manner to the solution and then stirred for 15 minutes. Then, 4-bromobenzaldehyde (1.57 g, 8.49 mmol) in DMF (3 mL) was added thereto in a slow manner and stirred for 18 hours while gradually heating it from 0° C. to room temperature. EtOAc was added to the reaction solution, washed with water and saturated saline and then dried over anhydrous MgSO₄. The solvent was eliminated in vacuo to obtain a residue.

The resulting residue was dissolved in THF (50 mL) and MeOH (20 mL), 1N NaOH (40 mL, 40 mmol) was added and then stirred at room temperature for 8 hours. The solvent was eliminated in vacuo, 2N HCl was added to acidify the solution and the precipitated crystals was filtrated to obtain white crystals of the objective carboxylic acid (729 mg, 35%).

MS: 242

<Step 2>

The carboxylic acid obtained from Step 1 (729 mg, 3.0 mmol) was dissolved in DMF (20 mL), CDI (535 g, 3.3 mmol) was added and then stirred at room temperature for 30 minutes. N-Boc-guanidine (720 mg, 4.5 mmol was added to the solution and then stirred for 16 hours. After the solvent was eliminated in vacuo, TFA (10 mL) was added to the residue at 0° C. and then stirred for 1.5 hours. After vacuum concentration of the solvent, it was purified by reversed phase HPLC (0.1% TFA in water/CH₃CN) to obtain the objective Intermediate 5 (348 mg, 29%).

### Intermediate 6

### Synthesis of N-[(E)-3-(4-bromo-2-methyl-phenyl)-2-methyl-acryloyl]-guanidine

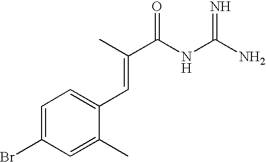 <Step 1>

The Intermediate 6 (330 mg, 17%) was obtained from 4-bromo-2-methylbenzaldehyde (1.0 g,) in the same manner as described for Intermediate 1.

MS: 297

### Example 1

### Synthesis of N-[(E)-3-(4′-chloro-biphenyl-2-yl)-2-methyl-acryloyl]-guanidine

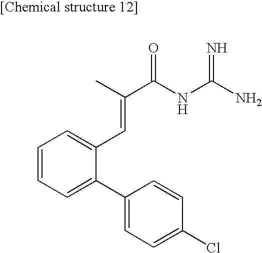 <Step 1>

Intermediate 1 (20 mg, 0.05 mmol) and 4-chlorophenylboronic acid (9 mg. 0.055 mmol were dissolved in a mixed solution of dioxane and water (v/v=3/1, 4.0 mL). Pd(PPh₃)₄ (3 mg, 2.6 μmol and Na₂CO₃ (21 mg, 0.2 mmol) were added to the solution and then stirred at 90° C. for 2 hours. After cooling it to room temperature, the solvent was eliminated in vacuo and then purified by reversed phase HPLC (0.1% TFA in water/CH₃CN) to obtain the compound of Example 1 (5.7 mg, 27%).

MS: 314

### Example 2

### Synthesis of N-[(E)-3-(4′-hydroxy-biphenyl-2-yl)-2-methyl-acryloyl]-guanidine

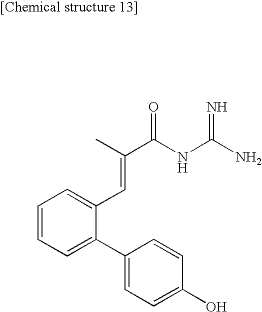 <Step 1>

Intermediate 1 (50 mg. 0.126 mmol) and 4-hydroxyphenylboronic acid (19.2 mg, 0.139 mmol were dissolved in a mixed solution of dioxane and water (v/v=3/1, 2.4 mL). Pd(PPh₃)₄ (7.29 mg, 6.30 μmol) and Na₂CO₃ (40.1 mg, 0.378 mmol) were added to the solution and then stirred at 90° C. for 15.5 hours. After cooling it to room temperature, the solvent was eliminated in vacuo and then purified by reversed phase HPLC (0.1% TFA in water/CH₃CN) to obtain the compound of Example 2 (51.6 mg, 100%).

1H-NMR (d-DMSO, 300 MHz), σ 2.01 (s, 3H), 6.82 (d, 2H, J=8.5 Hz), 7.13 (d, 2H, J=8.5 Hz), 7.33 (s, 1H), 7.37-7.52 (m, 4H), 8.19-8.33 (bs, 4H), 9.66 (s, 1H)

MS: 296

### Example 3

### Synthesis of N-[(E)-3-(4′-methoxy-biphenyl-2-yl)-2-methyl-acryloyl]-guanidine

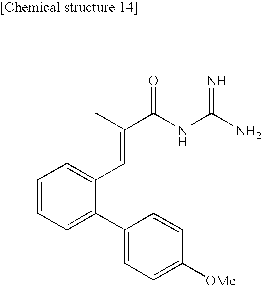 <Step 1>

Intermediate 1 (20 mg, 0.05 mmol) and 4-methoxyphenylboronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg. 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 3 (6.6 mg, 31%).

MS: 310

### Example 4

### Synthesis of N-[(E)-3-(4′-ethoxy-biphenyl-2-yl)-2-methyl-acryloyl]-guanidine

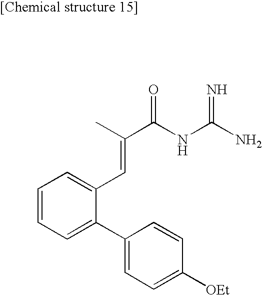 <Step 1>

2-bromobenzaldehyde (200 mg, 1.08 mmol) and 4-ethoxyphenylboronic acid (179 mg, 1.08 mmol) were dissolved in a mixed solution of dioxane and water (v/v=3/1, 8 mL). Pd(PPh₃)₄ (125 mg, 0.108 mmol) and Na₂CO₃ (343 mg, 3.24 mmol) were added to the solution and then stirred at 90° C. for 6 hours. After cooling it to room temperature, EtOAc was added thereto, washed with NaHCO₃ solution, water and saturate saline and then dried over anhydrous MgSO₄. The solvent was eliminated in vacuo and then purified by silica gel column chromatography (Hexane/EtOAc) to obtain the objective aldehyde (202 mg, 82.6%). 1H-NMR (d-DMSO, 300 MHz) σ 1.46 (t, 3H, J=7.0 Hz), 4.10 (q, 2H, J=7.0 Hz), 6.99 (d, 2H, J=8.5 Hz), 7.26 (s, 1H), 7.30 (d, 2H, J=8.5 Hz), 7.39-7.50 (m, 2H), 7.62 (ddd, 2H, J=1.5, 7.3, 7.3 Hz), 7.39-7.50 (m, 2H, J=1.1, 7.3 Hz), 10.0 (s, 1H)

MS: 227

<Step 2>

NaH (60% assay, 53.6 mg, 1.34 mmol) was suspended in THF (5 mL) and then cooled to 0° C. Triethyl 2-phosphonopropionate (319 mg, 1.34 mmol) in THF (2 mL) was added in a slow manner to the suspension. After stirring it for 15 minutes, the aldehyde obtained from Step 1 (202 mg, 0.893 mmol) in THF (2 mL) was added thereto and then stirred overnight while gradually heating it to room temperature. EtOAc was added thereto, washed with NaHCO₃, water and saturated saline, and then dried over anhydrous MgSO₄. After eliminating the solvent in vacuo, it was purified by silica gel column chromatography (Hexane/EtOAc) to obtain the objective ester (258 mg, 93.0%).

1H-NMR (d-DMSO, 300 MHz) σ 1.26 (t, 3H, J=7.0 Hz), 1.44 (t, 3H, J=7.0 Hz), 2.01 (s, 3H), 4.07 (q, 2H, J=7.0 Hz), 4.20 (q, 2H, J=7.0 Hz), 6.91 (d, 2H, J=8.8 Hz), 7.23 (d, 2H, J=8.8 Hz), 7.31 (s, 1H), 7.33-7.40 (m, 3H), 7.54 (bs, 1H), 7.98 (d, 1H, J=16 Hz)

MS: 311

<Step 3>

The ester obtained from Step 2 (258 mg, 0.831 mmol) was dissolved in a mixed solution of THF and MeOH (v/v=4/1, 5.2 mL). 2N NaOH (4.2 mL, 8.31 mmol) was added to the solution and then stirred at room temperature for 63.5 hours. After the solvent was eliminated in vacuo, 2N HCl (4.2 mL) was added thereto to acid the solution, EtOAc was added thereto, washed with water and saturated saline, and then dried over anhydrous MgSO₄. The solvent was eliminated in vacuo to obtain a quantitative amount of the objective carboxylic acid (235 mg).

MS: 283

The carboxylic acid obtained from Step 3 (50 mg, 0.177 mmol was dissolved in DMF (3 mL), CDI (31.6 mg, 0.195 mmol) was added thereto and then stirred at room temperature for 30 minutes, 2N guanidine solution in DMF (0.266 mL, 0.531 mmol) was added to the solution and then stirred at room temperature for 21 hours. After the solvent was eliminated in vacuo, it was purified by reversed phase HPLC (0.1% TFA in water/CH₃CN) to obtain the compound of Example 4 (2.2 mg, 2.84%).

MS: 324

### Example 5

### Synthesis if N-[(E)-3-(4′-acetyl-biphenyl-2-yl)-2-methyl-acryloyl]-guanidine

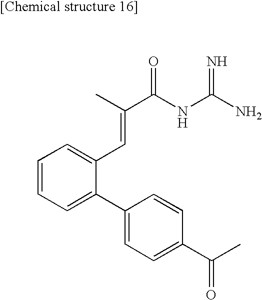 <Step 1>

Intermediate 1 (20 mg, 0.05 mmol) and 4-acetylphenylboronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 5 (6.8 mg, 31.0%).

MS: 322

### Example 6

### Synthesis of N-[(E)-3-(4′-hydroxymethyl-biphenyl-2-yl)-2-methyl-acryloyl]-guanidine

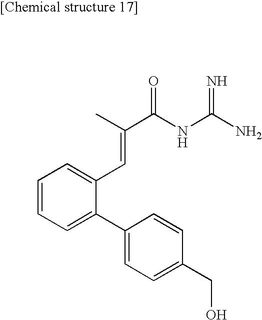 <Step 1>

Intermediate 1 (20 mg, 0.05 mmol) and 4-hydroxymethylphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 6 (7.8 mg, 38%).

MS: 310

### Example 7

### Synthesis of (E)-2′-(3-guanidino-2-methyl-3-oxo-propenyl)-biphenyl-4-carboxylic acid methyl ester

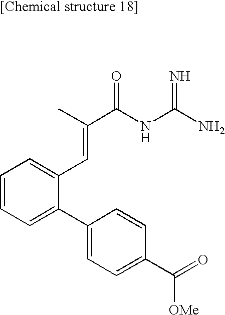 <Step 1>

Intermediate 1 (20 mg, 0.05 mmol) and 4-methoxycarbonylphenyl boronic acid (11 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 7 (3.3 mg, 15%).

MS: 338

### Example 8

### Synthesis of N-[(E)-2′-(3-guanidino-2-methyl-3-oxo-propenyl)-biphenyl-4-yl]-methane sulfonamide

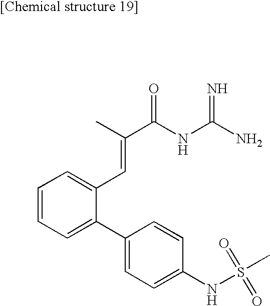 <Step 1>

Intermediate 1 (20 mg, 0.05 mmol) and 4-methanesulfonamidephenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 8 (9.9 mg, 41%).

MS: 373

### Example 9

### Synthesis of (E)-2′-(3-guanidino-2-methyl-3-oxo-propenyl)-biphenyl-4-carboxylic acid amide

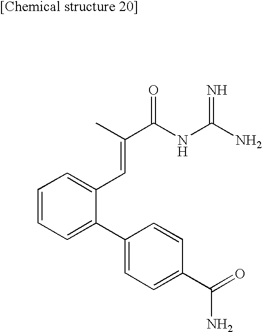 <Step 1>

Intermediate 1 (20 mg, 0.05 mmol) and 4-carboxyamidephenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 9 (3.1 mg, 14%).

MS: 323

### Example 10

### Synthesis of (E)-2′-(3-guanidino-2-methyl-3-oxo-propenyl)-biphenyl-4-carboxylic acid

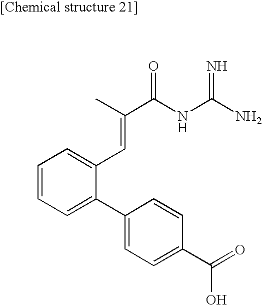 <Step 1>

Intermediate 1 (20 mg, 0.05 mmol) and 4-carboxyphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 10 (4.2 mg, 19%).

MS: 324

### Example 11

### Synthesis of N-[(E)-3-(4′-boronic acid-biphenyl-2-yl)-2-methyl-acryloyl]guanidine

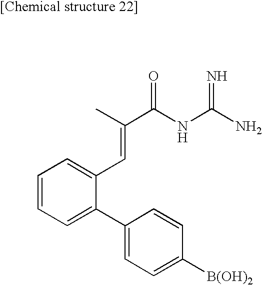 <Step 1>

Intermediate 1 (20 mg, 0.05 mmol) and 1,4-benzenediboronic acid (10 mg, 0.06 mmol) were dissolved in a mixed solution of dioxane and water (v/v=3/1, 2.4 mL). Pd(PPh₃)₄ (3.00 mg, 2.60 μmol) and Na₂CO₃ (21.0 mg, 0.152 mmol) were added to the solution and then stirred at 90° C. for 15.5 hours. After cooling it to room temperature, the solvent was eliminated in vacuo and then purified by reversed phase HPLC (0.1% TFA in water/CH₃CN) to obtain the compound of Example 11 (5.0 mg, 23%).

MS: 324

### Example 12

### Synthesis of N-[(E)-2-methyl-3-(4′-nitro-biphenyl-2-yl)-acryloyl]guanidine

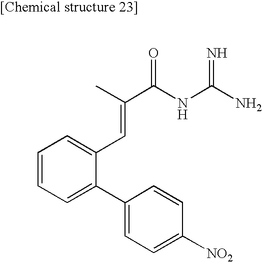 <Step 1>

Intermediate 1 (20 mg, 0.05 mmol) and 4-nitrophenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 12 (3.8 mg, 17%).

MS: 325

### Example 13

### Synthesis of N-[(E)-3-(3′-hydroxy-biphenyl-2-yl)-2-methyl-acryloyl]guanidine

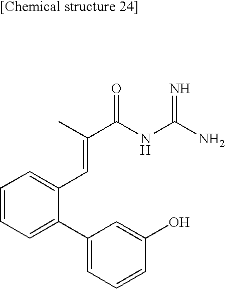 <Step 1>

Intermediate 1 (20 mg, 0.05 mmol) and 3-hydroxyphenylboronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 13 (5.6 mg, 27%).

MS: 296

### Example 14

### Synthesis of N-{(E)-3-[3′-(1-hydroxy-ethyl)-biphenyl-2-yl]-2-methyl-acryloyl}guanidine

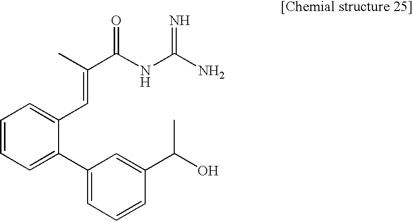 <Step 1>

The compound of Example 15 (10 mg, 0.0229 mmol) was dissolved in THF (1 mL), cooled to 0° C., NaBH₄ (2 mg, 0.046 mmol) was added thereto and then stirred at room temperature for 2 hours. After the solvent was eliminated in vacuo, it was purified by reversed phase HPLC (0.1% TFA in water/CH₃CN) to obtain the compound of Example 14 (4.0 mg, 40%).

MS: 324

### Example 15

### Synthesis of N-[(E)-3-(3′-acetyl-biphenyl-2-yl)-2-methyl-acryloyl]guanidine

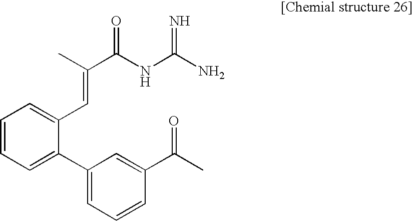 <Step 1>

Intermediate 1 (20 mg, 0.05 mmol) and 4-acetylphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 15 (4.3 mg, 20%).

MS: 322

### Example 16

### Synthesis of (E)-2′-(3-guanidino-2-methyl-3-oxo-propenyl)-biphenyl-3-carboxylic acid methyl ester

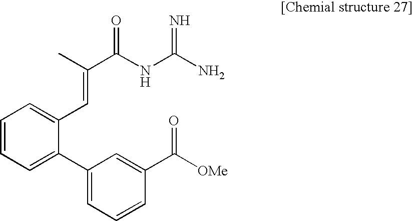 <Step 1>

Intermediate 1 (20 mg, 0.05 mmol) and 3-methoxycarbonylphenylboronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 16 (3.3 mg, 31%).

MS: 338

### Example 17

### Synthesis of N-[(E)-2′-(3-guanidino-2-methyl-3-oxo-propenyl)-biphenyl-3-yl]methanesulfonamide

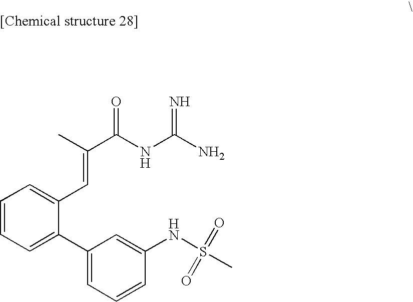 <Step 1>

Intermediate 1 (20 mg, 0.05 mmol) and 3-methanesulfonamidephenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred overnight at 90° C. After cooling it to room temperature, the solvent was eliminated in vacuo and then purified by reversed phase HPLC (0.1% TFA in water/CH₃CN) to obtain the compound of Example 17 (6.3 mg, 27%).

MS: 373

### Example 18

### Synthesis of (E)-2′-(3-guanidino-2-methyl-3-oxo-propenyl)-biphenyl-3-carboxylic acid amide

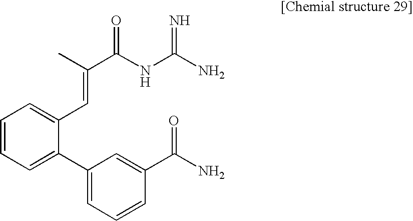 <Step 1>

Intermediate 1 (20 mg, 0.05 mmol) and 3-carboxyamidephenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 18 (4.6 mg, 21%).

MS: 323

### Example 19

### Synthesis of (E)-2′-(3-guanidino-2-methyl-3-oxo-propenyl)-biphenyl-3-carboxylic acid

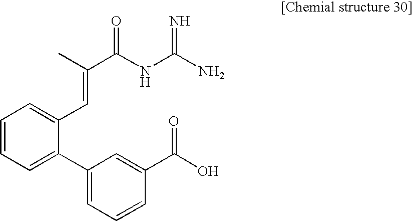 <Step 1>

Intermediate 1 (20 mg, 0.05 mmol) and 3-carboxyphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 19 (6.5 mg, 30%).

MS: 324

### Example 20

### Synthesis of N-[(E)-3-(3′-cyano-biphenyl-2-yl)-2-methyl-acryloyl]-guanidine

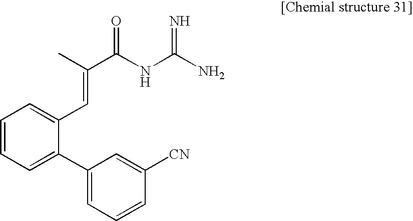 <Step 1>

Intermediate 1 (20 mg, 0.05 mmol) and 3-cyanophenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 20 (4.1 mg, 20%).

MS: 305

### Example 21

### Synthesis of N-[(E)-3-(2′-hydroxy-biphenyl-2-yl)-2-methyl-acryloyl]-guanidine

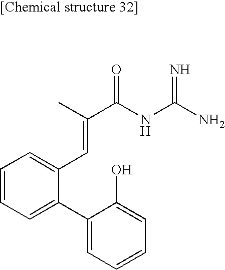 <Step 1>

Intermediate 1 (20 mg, 0.05 mmol) and 2-hydroxyphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 21 (5.6 mg, 27%).

MS: 296

### Example 22

### Synthesis of N-[(E)-3-(3′,5′-dimethyl-4′-hydroxy-biphenyl-2-yl)-2-methyl-acryloyl]-guanidine

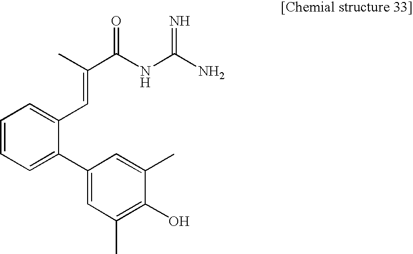 <Step 1>

Intermediate 1 (20 mg, 0.05 mmol) and 3,5-dimethyl-4-hydroxyphenyl boronic acid (10 mg, 0.06 mmol) were dissolved in a mixed solution of dioxane and water (v/v=3/1, 2.4 mL). Pd(PPh₃)₄ (7.29 mg, 6.30 μmol) and Na₂CO₃ (40.1 mg, 0.378 mmol) were added to the solution and then stirred at 90° C. for 15.5 hours. After cooling it to room temperature, the solvent was eliminated in vacuo and then purified by reversed phase HPLC (0.1% TFA in water/CH₃CN) to obtain the compound of Example 22 (5.0 mg, 23%).

MS: 324

### Example 23

### Synthesis of N-[(E)-3-(4′-hydroxy-3′-methoxy-biphenyl-2-yl)-2-methyl-acryloyl]guanidine

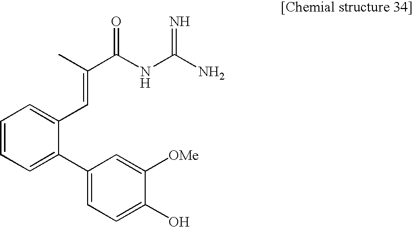 <Step 1>

Intermediate 1 (20 mg, 0.05 mmol) and 3-methoxy-4-hydroxyphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol were added to the solution and then stirred at 90° C. overnight. After cooling it to room temperature, the solvent was eliminated in vacuo and then purified by reversed phase HPLC (0.1% TFA in water/CH₃CN) to obtain the compound of Example 23 (5.2 mg, 24%).

MS: 326

### Example 24

### Synthesis of N-[(E)-3-(3′-fluoro-4′-hydroxy-biphenyl-2-yl)-2-methyl-acryloyl]-guanidine

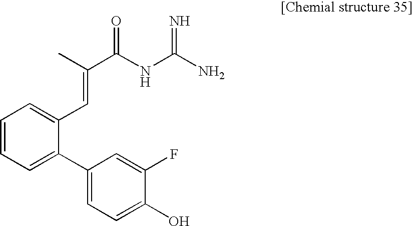 <Step 1>

Intermediate 1 (20 mg, 0.05 mmol) and 3-fluoro-4-hydroxyphenyl boronic acid (10.2 mg, 0.06 mmol) were dissolved in a mixed solution of dioxane and water (v/v=3/1, 2.4 mL). Pd(PPh₃)₄ (3.00 mg, 2.60 μmol) and Na₂CO₃ (21.0 mg, 0.152 mmol) were added to the solution and then stirred at 90° C. for 15.5 hours. After cooling it to room temperature, the solvent was eliminated in vacuo and then purified by reversed phase HPLC (0.1% TFA in water/CH₃CN) to obtain the compound of Example 24 (3.0 mg, 14%).

MS: 314

### Example 25

### Synthesis of N-[(E)-3-(3′,5′-difluoro-4′-hydroxy-biphenyl-2-yl)-acryloyl]-guanidine

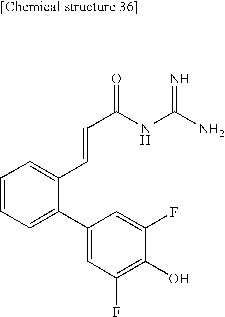 <Step 1>

The intermediate obtained from Step 2 of Example 32 (20 mg, 0.05 mmol) and 3,5-difluoro-4-hydroxyphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 25 (5.2 mg, 24%).

MS: 318

### Example 26

### Synthesis of N-[(E)-3-(3′,4′-dihydroxy-biphenyl-2-yl)-2-methyl-acryloyl]-guanidine

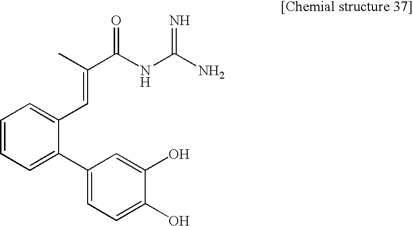 <Step 1>

Intermediate 1 (20 mg, 0.05 mmol) and 3,4-dihydroxyphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 26 (5.3 mg, 25%).

MS: 312

### Example 27

### Synthesis of N-[(E)-3-(3′,5′-dihydroxy-biphenyl-2-yl)-2-methyl-acryloyl]-guanidine

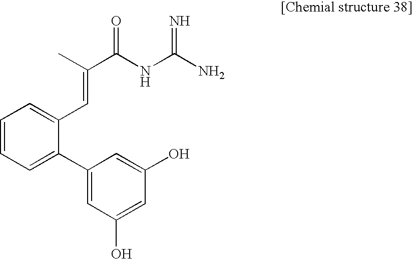 <Step 1>

Intermediate 1 (20 mg, 0.05 mmol) and 3,5-dihydroxyphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 27 (6.1 mg, 29%).

MS: 312

### Example 28

### Synthesis of N-[(E)-3-(3′,4′,5′-trihydroxy-biphenyl-2-yl)-2-methyl-acryloyl]-guanidine

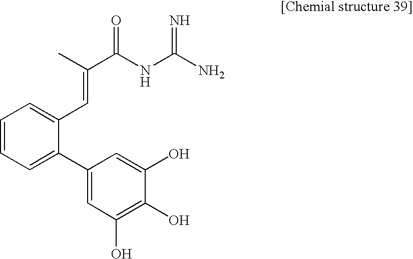 <Step 1>

Intermediate 1 (20 mg, 0.05 mmol) and 3,4,5-trihydroxyphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 28 (3.5 mg, 16%).

MS: 328

### Example 29

### Synthesis of N-[(E)-3-(4′-hydroxy-2′-methyl-biphenyl-2-yl)-2-methyl-acryloyl]-guanidine

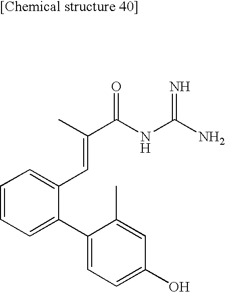 <Step 1>

Intermediate 1 (100 mg, 0.253 mmol) and 2-methyl-4-methoxyphenyl boronic acid (46.1 mg, 0.278 mmol) were dissolved in a mixed solution of dioxane and water (v/v=3/1, 3 mL). Pd(PPh₃)₄ (14.6 mg, 12.7 μmol) and Na₂CO₃ (80.5 mg, 0.759 mmol) were added to the solution and then stirred at 90° C. for 2.5 hours. After cooling it to room temperature, the solvent was eliminated in vacuo and then purified by reversed phase HPLC (0.1% TFA in water/CH₃CN) to obtain a coupling product (69.7 mg, 63%).

MS: 324

<Step 2>

CH₂Cl₂ (2.0 mL) was added to the coupling product obtained from Step 1 (25 mg, 0.057 mmol) to dissolve, 1.0 mol/L BBr₃ dichloromethane solution (0.35 mL, 0.35 mmol) was added to the solution and then stirred at room temperature for 3 hours. After concentrating the solvent in vacuo, it was purified by reversed phase HPLC (0.1% TFA in water/CH₃CN) to obtain the objective compound of Example 29 (16.7 mg, 68.9%).

MS: 310

### Example 30

### Synthesis of N-[(E)-3-(2-benzo[1,3]dioxole-5-yl-phenyl)-2-methyl-acryloyl]-guanidine

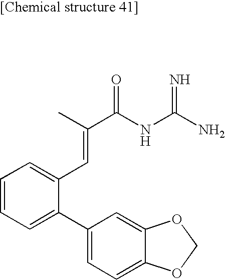 <Step 1>

Intermediate 1 (20 mg, 0.05 mmol) and 3,4-methylenedioxyphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 30 (5.4 mg, 25%).

MS: 324

### Example 31

### Synthesis of N-{(E)-3-[2-(2,3-dihydro-benzo[1,4]dioxin-6-yl)-phenyl]-2-methyl-acryloyl}-guanidine

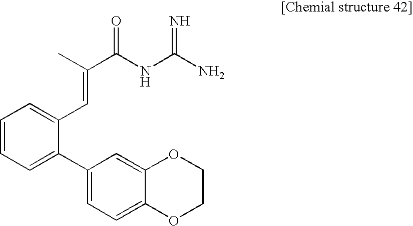 <Step 1>

Intermediate 1 (20 mg, 0.05 mmol) and 3,4-ethylenedioxyphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 31 (12 mg, 53%).

MS: 338

### Example 32

### Synthesis of N-[(E)-3-(4′-hydroxy-biphenyl-2-yl)-acryloyl]-guanidine

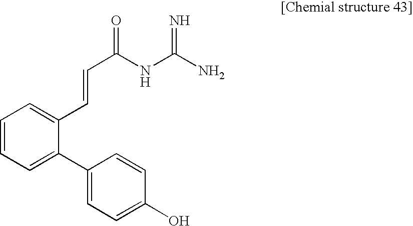 <Step 1>

2-bromobenzaldehyde (500 mg, 2.70 mmol) and malonic acid (562 mg, 5.40 mmol) were dissolved in pyridine (5 mL). Pyrrolidine (19.2 mg, 0.270 mmol) was added to the solution and then stirred at 100° C. for 19.5 hours. After cooling it to room temperature, the solvent was eliminated in vacuo and then white crystals of the objective carboxylic acid (402 mg, 65.5%) were obtained by decantation.

1H-NMR (d-DMSO, 300 MHz) σ 6.57 (d, 2H, J=15.8 Hz), 7.36 (ddd, 1H, J=1.8, 7.6, 7.6 Hz), 7.44 (ddd, 1H, J=1.2, 7.6, 7.6 Hz), 7.71 (dd, 1H, J=1.2, 7.6 Hz), 7.84 (d, 1H, 15.8 Hz), 7.90 (dd, J=1.8, 7.6 Hz)

MS: 241

<Step 2>

The carboxylic acid obtained from Step 1 (402 mg, 1.77 mmol) was dissolved in DMF (15 mL), CDI (287 mg, 1.77 mmol) was added thereto and then stirred at room temperature for 30 minutes. N-Boc-guanidine (338 mg, 2.13 mmol) was added to the solution and then stirred for 19.5 hours. Then, after the solvent was eliminated in vacuo, TFA (5 mL) was added to the residue and then stirred at room temperature for 6 hours. The solvent was eliminated in vacuo and then purified by reversed phase HPLC (0.1% TFA in water/CH₃CN) to obtain the objective acylguanidine (308 mg, 45.5%).

1H-NMR (d-DMSO, 300 MHz) σ 6.78 (d, 1H, J=16 Hz), 7.43 (ddd, 1H, J=1.8, 7.67.6 Hz), 7.52 (ddd, 1H, J=1.2, 7.6, 7.6 Hz), 7.73-7.84 (m, 2H), 8.39 (bs, 1H)

MS: 268

<Step 3>

The acylguanidine obtained from Step 2 (50 mg, 0.131 mmol) and 4-hydroxyphenyl boronic acid (19.9 mg, 0.144 mmol) was dissolved in a mixed solution of dioxane and water (v/v=3/1, 3 mL). Pd(PPh₃)₄ (7.58 mg, 6.60 μmol) and Na₂CO₃ (41.7 mg, 0.393 mmol) were added to the solution and then stirred at 90° C. for 18.5 hours. After cooling it to room temperature, the solvent was eliminated in vacuo, purified by reversed phase HPLC (0.1% TFA in water/CH₃CN) and then repurified by silica gel column chromatography (amino, CH₂Cl₂/MeOH) to obtain the compound of Example 32 (4.14 mg, 7.99%).

MS: 282

### Example 33

### Synthesis of N-[(E)-3-(3′-hydroxy-biphenyl-2-yl)-acryloyl]-guanidine

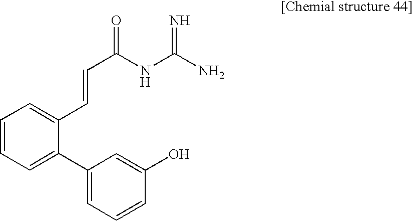 <Step 1>

The Acylguanidine obtained from Step 2 in Example 32 (50 mg, 0.131 mmol) and 3-(4,4,5,5-tetramethyl-1,3,2-dioxaborolane-2-yl)phenol (31.7 mg, 0.144 mmol) were dissolved in a mixed solution of dioxane and water (v/v=3/1, 3 mL). Pd(PPh₃)₄ (7.58 mg, 6.60 μmol) and Na₂CO₃ (41.7 mg, 0.393 mmol) were added to the solution and then stirred at 90° C. for 18.5 hours. After cooling it to room temperature, the solvent was eliminated in vacuo and then purified by reversed phase HPLC (0.1% TFA in water/CH₃CN) to obtain the compound of Example 33 (18.2 mg, 7.99%).

1H-NMR (d-DMSO, 300 MHz) σ 6.67-6.77 (m, 2H), 6.81-6.88 (m, 1H), 7.24-7.32 (m, 1H), 7.40 (dd, 1H, J=1.8, 7.0 Hz), 7.47-7.59 (m, 2H), 7.70 (d, 1H, J=16 Hz), 7.77-7.83 (m, 1H), 8.24-8.48 (bs, 4H)

MS: 282

### Example 34

### Synthesis of N-[(E)-3-(3′-hydroxy-biphenyl-2-yl)-2-ethyl-acryloyl]-guanidine

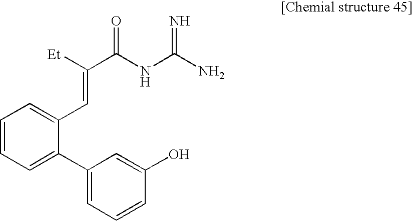 <Step 1>

NaH (97.3 mg, 2.43 mmol) was suspended in THF (10 mL) and then cooled to 0° C. 2-phosphono butyric acid triethyl (613 mg, 2.43 mmol) in THF (3 mL) was added dropwise in a slow manner to the solution and then stirred for 15 minutes. Then, 2-bromobenzaldehyde (300 mg, 1.62 mmol) in THF (3 mL) was added thereto in a slow manner and then stirred for 18 hours while gradually heating it from 0° C. to room temperature. EtOAc was added to the reaction solution, washed with water and saturated saline and then dried over anhydrous MgSO₄. The solvent was eliminated in vacuo to obtain a residue. The residue obtained was dissolved in THF (10 mL) and MeOH (2 mL), 2 N NaOH (4 mL, 8.0 mmol) was added and then stirred at 50° C. for 8 hours. The solvent was eliminated in vacuo, 2N HCl was added to acidify the solution and then the precipitated crystals were filtrated to obtain white crystals of the objective carboxylic acid (364 mg, 88.4%).

1H-NMR (d-DMSO, 300 MHz) σ 1.13 (t, 3H, J=7.3 Hz), 2.40 (q, 2H, J=7.3 Hz), 7.16-7.39 (m, 4H), 7.63 (d, 1H, J=8.2 Hz), 7.78 (s, 1H)

MS: 255

<Step 2>

The carboxylic acid obtained from Step 2 (250 mg, 0.984 mmol) was dissolved in DMF (10 mL), CDI (191 mg, 1.18 mmol) was added thereto and then stirred at room temperature for 30 minutes. N-Boc-guanidine (188 mg, 1.18 mmol) was added to the solution and then stirred for 16 hours. After the solvent was eliminated in vacuo, TFA (4 mL) was added to the residue at 0° C. and then stirred for 1.5 hours. After concentrating the solvent in vacuo, it was purified by reversed phase HPLC (0.1% TFA in water/CH₃CN) to obtain the objective acylguanidine (228 mg, 56.6%).

MS: 296

The acylguanidine obtained from Step 2 (42.5 mg, 0.104 mmol) and 3-(4,4,5,5-tetramethyl-1,3,2-dioxaborolane-2-yl)phenol (34.3 mg, 0.156 mmol) were dissolved in a mixed solution of dioxane and water (v/v=3/1, 2.0 mL). Pd(PPh₃)₄ (6.01 mg, 5.20 μmol) and Na₂CO₃ (33.1 mg, 0.312 mmol) were added to the solution and then stirred at 90° C. for 21.5 hours. After cooling it to room temperature, the solvent was eliminated in vacuo and then purified by reversed phase HPLC (0.1% TFA in water/CH₃CN) to obtain the compound of Example 34 (28.3 mg, 64.3%).

1H-NMR (d-DMSO, 300 MHz) σ 1.05 (t, 3H, J=7.3 Hz), 2.44-2.57 (m, 2H), 6.71-6.84 (m, 3H), 7.21-7.30 (m, 2H), 7.41-7.56 (m, 4H), 8.13-8.50 (bs, 4H)

MS: 310

### Example 35

### Synthesis of N-[(E)-3-(4,3′-dihydroxy-biphenyl-2-yl)-2-methyl-acryloyl]-guanidine

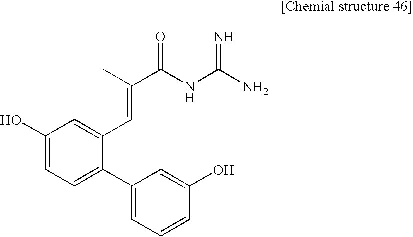 <Step 1>

The objective acylguanidine was obtained from 2-bromo-5-methoxybenzaldehyde in the same manner as described for Intermediate 1.

<Step 2>

The intermediate obtained from Step 1 (20 mg, 0.05 mmol) and 3-hydroxyphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain a coupling product (5.4 mg, 25%).

MS: 328

<Step 3>

The coupling product obtained from Step 2 (10 mg, 0.023 mmol) was dissolved in CH₂Cl₂ (1 mL), cooled to 0° C., 1.0 mol/L BBr₃ dichloromethane solution (0.34 mL, 0.341 mmol) was added thereto and stirred at room temperature for 2 hours. After the solvent was eliminated in vacuo, it was purified by reversed phase HPLC (0.1% TFA in water/CH₃CN) to obtain the compound of Example 35 (29.1 mg, 79%).

MS: 312

### Example 36

### Synthesis of N-[(E)-2-methyl-3-(4,5,3′-trihydroxy-biphenyl-2-yl)-acryloyl]-guanidine

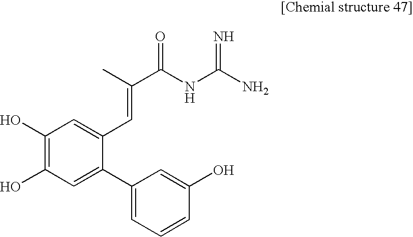 <Step 1>

The compound of Example 39 (20 mg, 0.045 mmol) was dissolved in CH₂Cl₂ (1 mL), cooled to 0° C., 1.0 mol/L BBr₃ dichloromethane solution (0.34 mL, 0.341 mmol) was added thereto and then stirred at room temperature for 2 hours. After the solvent was eliminated in vacuo, it was purified by reversed phase HPLC (0.1% TFA in water/CH₃CN) to obtain the compound of Example 36 (10.8 mg, 54%).

MS: 328

### Example 37

### Synthesis of N-[(E)-3-(3′-hydroxy-5-methyl-biphenyl-2-yl)-2-methyl-acryloyl]-guanidine

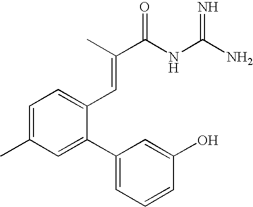 <Step 1>

Intermediate 2 (20 mg, 0.05 mmol) and 3-hydroxyphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 37 (5 mg, 24%).

MS: 310

### Example 38

### Synthesis of N-[(E)-3-(3′-hydroxy-4-methyl-biphenyl-2-yl)-2-methyl-acryloyl}-guanidine

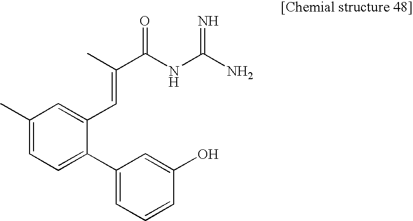 <Step 1>

An acylguanidine which is an intermediate was obtained from 2-bromo-5-methylbenzaldehyde in the same manner as described for Intermediate 1.

<Step 2>

The intermediate obtained from Step 1 (20 mg, 0.05 mmol) and 3-hydroxyphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 38 (4.8 mg, 23%).

MS: 310

### Example 39

### Synthesis of N-[(E)-3-(3′-hydroxy-4,5-dimethoxy-biphenyl-2-yl)-2-methyl-acryloyl]-guanidine

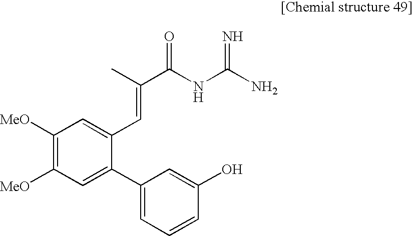 <Step 1>

An acylguanidine which is an intermediate was obtained from 2-bromo-4,5-dimethoxybenzaldehyde in the same manner as described for Intermediate 1.

<Step 2>

The intermediate obtained from Step 1 (20 mg, 0.05 mmol) and 3-hydroxyphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 39 (4.9 mg, 21%).

MS: 356

### Example 40

### Synthesis of N-[(E)-3-(2,6-di(3-hydroxyphenyl)-phenyl)-2-methyl-acryloyl]guanidine

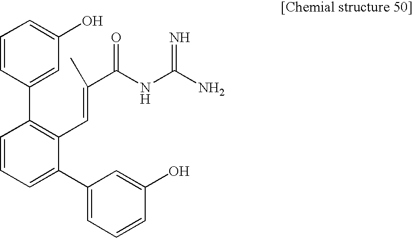 <Step 1>

Intermediate 3 (40 mg, 0.08 mmol) and 3-hydroxyphenylboronic acid (18 mg, 0.11 mmol) were dissolved in a mixed solution of dioxane and water (v/v=3/1, 4.0 mL). Pd(PPh₃)₄ (6 mg, 5.2 μmol) and Na₂CO₃ (42 mg, 0.4 mmol) were added to the solution and then stirred at 90° C. for 2 hours. After cooling it to room temperature, the solvent was eliminated in vacuo and then purified by reversed phase HPLC (0.1% TFA in water/CH₃CN) to obtain the compound of Example 40 (5.8 mg, 14%).

MS: 388

### Example 41

### Synthesis of N-[(E)-3-(2,5-di(3-hydroxyphenyl)-phenyl)-2-methyl-acryloyl]-guanidine

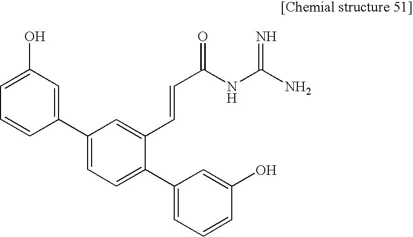 <Step 1>

An acylguanidine which is an intermediate was obtained from 2,5-dibromobenzaldehyde in the same manner as described for Intermediate 1.

<Step 2>

The intermediate obtained from Step 1 (40 mg, 0.08 mmol) and 3-chlorophenyl boronic acid (18 ml, 0.11 mmol) were dissolved in a mixed solution of dioxane and water (v/v=3/1, 4.0 mL). Pd(PPh₃)₄ (6 mg, 5.2 μmol) and Na₂CO₃ (42 mg, 0.4 mmol) were added to the solution and then stirred at 90° C. for 2 hours. After cooling it to room temperature, the solvent was eliminated in vacuo and then purified by reversed phase HPLC (0.1% TFA in water/CH₃CN) to obtain the compound of Example 41 (3.7 mg, 9%).

MS: 388

### Example 42

### Synthesis of N-[(E)-3-(5-fluoro-3′-hydroxy-biphenyl-2-yl)-2-methyl-acryloyl]-guanidine

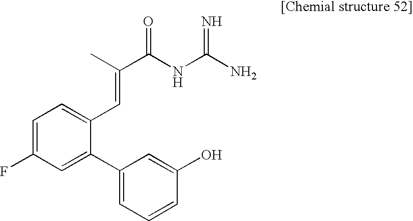 <Step 1>

An acylguanidine which is an intermediate was obtained from 2-bromo-4-fluorobenzaldehyde in the same manner as described for Intermediate 1.

<Step 2>

The intermediate obtained from Step 1 (20 mg, 0.05 mmol) and 3-hydroxyphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 42 (10.8 mg, 51%).

MS: 314

### Example 43

### Synthesis of N-[(E)-3-(5-fluoro-4′-hydroxy-biphenyl-2-yl)-2-methyl-acryloyl]-guanidine

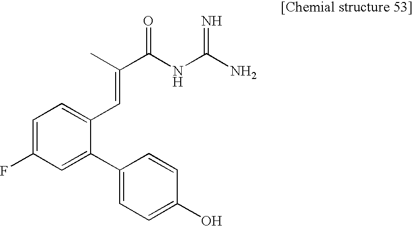 <Step 1>

The intermediate obtained from Step 1 in Example 42 (20 mg, 0.05 mmol) and 4-hydroxyphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 43 (10.2 mg, 50%).

MS: 314

### Example 44

### Synthesis of N-[(E)-3-(4,4′-dihydroxy-biphenyl-2-yl)-2-methyl-acryloyl]-guanidine

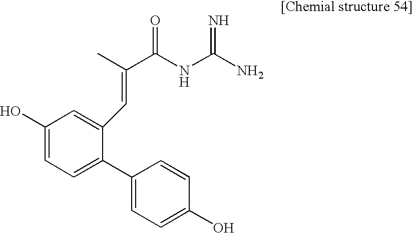 <Step 1>

The intermediate obtained from Step 1 in Example 35 (20 mg, 0.05 mmol) and 4-hydroxyphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain a coupling product (4.3 mg, 20%).

MS: 328

<Step 2>

The coupling product obtained from Step 1 (10 mg, 0.023 mmol) was dissolved in CH₂Cl₂ (1 mL), cooled to 0° C., 1.0 mol/L BBr₃ dichloromethane solution (0.34 mL, 0.341 mmol) was added thereto and then stirred at room temperature for 2 hours. After the solvent was eliminated in vacuo, it was purified by reversed phase HPLC (0.1% TFA in water/CH₃CN) to obtain the compound of Example 44 (29.1 mg, 40%).

MS: 312

### Example 45

### Synthesis of N-[(E)-3-(4′-hydroxy-4,5-dimethoxy-biphenyl-2-yl)-2-methyl-acryloyl]-guanidine

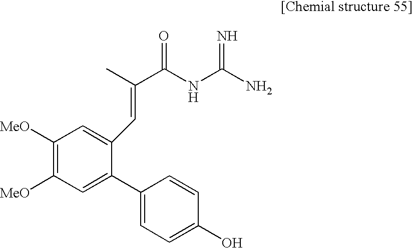 <Step 1>

The intermediate obtained from Step 1 in Example 39 (20 mg, 0.05 mmol) and 4-hydroxyphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 45 (5.1 mg, 22%).

MS: 356

### Example 46

### Synthesis of N-[(E)-2-methyl-3-(4,5,4′-trihydroxy-biphenyl-2-yl)-acryloyl]-guanidine

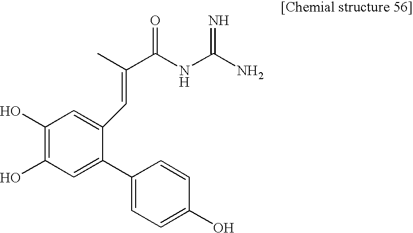 <Step 1>

The compound of Example 45 (20 mg, 0.045 mmol) was dissolved in CH₂Cl₂ (1 mL), cooled to 0° C., 1.0 mol/L BBr₃ dichloromethane solution (0.34 mL, 0.341 mmol) was added thereto and then stirred at room temperature for 2 hours. After the solvent was eliminated in vacuo, it was purified by reversed phase HPLC (0.1% TFA in water/CH₃CN) to obtain the compound of Example 46 (10.8 mg, 24%).

MS: 328

### Example 47

### Synthesis of N-[(E)-3-(4′-hydroxy-5-methyl-biphenyl-2-yl)-2-methyl-acryloyl]-guanidine

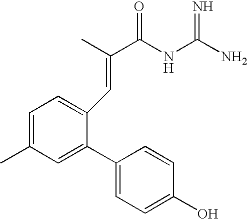 <Step 1>

Intermediate 2 (20 mg, 0.05 mmol) and 4-hydroxyphenyl boronic acid (10 mg, 0.06 mmol) were dissolved in a mixed solution of dioxane and water (v/v=3/1, 3 mL). Pd(PPh₃)₄ (3.00 mg, 2.60 μmol) and Na₂CO₃ (21.0 mg, 0.2 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 47 (8.1 mg, 38%).

MS: 310

### Example 48

### Synthesis of N-[(E)-3-(2,5-di(4-hydroxyphenyl)-phenyl)-2-methyl-acryloyl]-guanidine

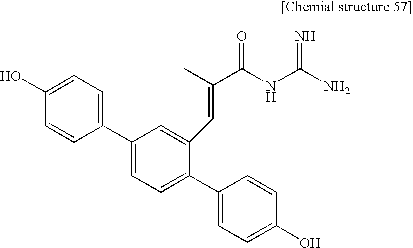 <Step 1>

The intermediate obtained from Step 1 in Example 41 and 4-hydroxyphenyl boronic acid were reacted to obtain the compound of Example 48 (7.4 mg, 18%) in the same manner as described in Example 41.

MS: 388

### Example 49

### Synthesis of N-{(E)-3-[2-(3-methoxy-phenoxy)-phenyl]-2-methyl-acryloyl}-guanidine

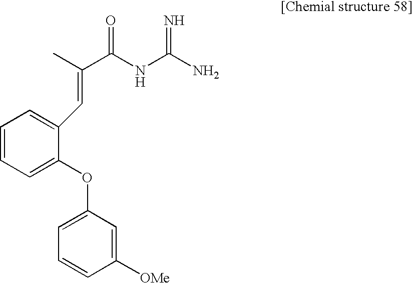 <Step 1>

2-fluorobenzaldehyde (100 mg, 0.806 mmol) and 3-methoxyphenol (110 mg, 0.886 mmol) were dissolved in DMA (4 mL), K₂CO₃ (335 mg, 2.42 mmol) was added thereto and then stirred at 170° C. for 1.5 hours. After cooling it to room temperature, it was purified by silica gel column chromatography (Hexane/EtOAc) to obtain the objective aldehyde (110 mg, 59.8%).

1H-NMR (d-DMSO, 300 MHz) σ 3.80 (s, 3H), 6.59-6.66 (m, 2H), 6.73 (ddd, 1H, J=1.2, 2.4, 8.2 Hz), 6.95 (d, 1H, J=8.2 Hz), 7.20 (dd, 1H, J=7.3, 8.5 Hz), 7.28 (dd, 1H, J=8.5, 8.5 Hz), 7.52 (ddd, 1H, J=1.8, 7.3, 8.5 Hz), 7.94 (dd, 1H, J=1.8, 7.9 Hz), 10.5 (s, 1H)

MS: 229

<Step 2>

NaH (60% assay, 28.9 mg, 0.723 mmol) was suspended in THF (5 mL) and then cooled to 0° C. Triethyl 2-phosphonopropionate (182 mg, 0.723 mmol) in THF (2 mL) was added dropwise in a slow manner to the solution and then stirred for 15 minutes. Then, the aldehyde obtained from Step 1 (110 mg, 0.482 mmol) in THF (1 mL) was added thereto in a slow manner and then stirred for 22 hours while gradually heating it from 0° C. to room temperature. EtOAc was added to the reaction solution, washed with water and saturated saline and then dried over anhydrous MgSO₄. The solvent was eliminated in vacuo to obtain a residue. The residue obtained was dissolved in THF (4 mL) and MeOH (2 mL), 2N NaOH (2 mL, 4.0 mmol) was added thereto and then stirred at 50° C. for 22 hours. The solvent was eliminated in vacuol, 2 N HCl was added to acidify the solution and then crystals precipitated were filtrated to obtain white crystals of the objective carboxylic acid (114 mg, 83.1%).

MS: 285

<Step 3>

The carboxylic acid obtained from Step 2 (114 mg, 0.400 mmol) was dissolved in DMF (4 mL), CDI (77.8 mg, 0.480 mmol) was added thereto and then stirred at room temperature for 30 minutes. N-Boc-guanidine (76.4 mg, 0.480 mmol) was added to the solution and then stirred for approximately 3 days. After the solvent was eliminated in vacuo, TFA (3 mL) was added to the residue and then stirred for 2.5 hours. After the solvent was eliminated in vacuo, it was purified by reversed phase HPLC (0.1% TFA in water/CH₃CN) to obtain the compound of Example 49 (97.8 mg, 55.3%).

MS: 326

### Example 50

### Synthesis of N-{(E)-3-[2-(4-hydroxy-phenoxy)-phenyl]-2-methyl-acryloyl}-guanidine

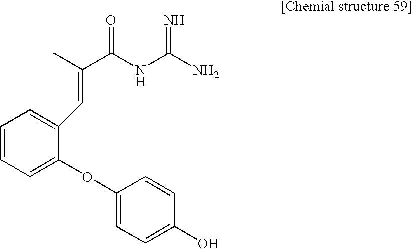 <Step 1>

2-fluorobenzaldehyde (100 mg, 0.806 mmol) and 4-methoxyphenol (110 mg, 0.886 mmol) were dissolved in DMA (4 mL), K₂CO₃ (335 mg, 2.42 mmol) was added thereto and then stirred at 170° C. for 2.5 hours. After cooling it to room temperature, it was purified by silica gel column chromatography (Hexane/EtOAc) to obtain the objective aldehyde which was a crude product (185 mg).

MS: 243

<Step 2>

NaH (60% assay, 48.6 mg, 1.22 mmol) was suspended in THF (5 mL) and then cooled to 0° C. 2-phosphonopropionic acid triethyl (307 mg, 1.22 mmol) in THF (2 mL) was added dropwise in a slow manner to the solution and then stirred for 30 minutes. Then, the aldehyde obtained from Step 1 (185 mg, 0.810 mmol) in THF (1 mL) was added in a slow manner and stirred for 14.5 hours while gradually heating it from 0° C. to room temperature. EtOAc was added to the reaction solution, washed with water and saturated saline and then dried over anhydrous MgSO₄. The solvent was then eliminated in vacuo to obtain a residue. The resulting residue was dissolved in THF (4 mL) and MeOH (2 mL), 2 N NaOH (2 mL, 4.0 mmol) was added thereto and then stirred at 50° C. for 5 hours. The solvent was eliminated in vacuo, 2 N HCl was added to acidify the solution and then the crystals precipitated were filtrated to obtain white crystals of the objective carboxylic acid (217 mg, 94.1% from Step 1).

MS: 285

<Step 3>

The carboxylic acid obtained from Step 2 (100 mg, 0.352 mmol) was dissolved in DMF (3 mL), CDI (68.4 mg, 0.422 mmol) was added thereto and then stirred at room temperature for 30 minutes. N-Boc-guanidine (67.1 mg, 0.422 mmol) was added to the solution and then stirred for approximately 3 days. After the solvent was eliminated in vacuo, TFA (2 mL) was added to the residue at 0° C. and then stirred for 2.5 hours. After the solvent was eliminated in vacuo, it was purified by reversed phase HPLC (0.1% TFA in water/CH₃CN) to obtain the objective acylguanidine (52.4 mg, 33.9%).

1H-NMR (d-DMSO, 300 MHz) σ 2.01 (s, 3H), 3.69 (s, 3H), 6.72 (d, 1H, J=8.2 Hz), 6.88-6.97 (m, 4H), 7.112 (dd, 1H, J=7.8, 8.2 Hz), 7.31 (ddd, 1H, J=1.5, 7.8, 8.5 Hz), 7.44 (dd, 1H, J=1.2, 7.8 Hz), 7.58 (s, 1H), 8.11-8.55 (bs, 4H)

MS: 326

<Step 4>

The compound obtained from Step 3 (30 mg, 0.0683 mmol) was dissolved in CH₂Cl₂ (1 mL), cooled to 0° C., 1.0 mol/L BBr₃ in dichloromethane (0.34 mL, 0.341 mmol) was added thereto and then stirred at room temperature for 2 hours. After the solvent was eliminated in vacuo, it was purified by reversed phase HPLC (0.1% TFA in water/CH₃CN) to obtain the compound of Example 50 (29.1 mg, 100%)

MS: 312

### Example 51

### Synthesis of N-[2′-(3-guanidino-2-methyl-3-oxo-propenyl)-biphenyl-2-yl]-methanesulfonamide

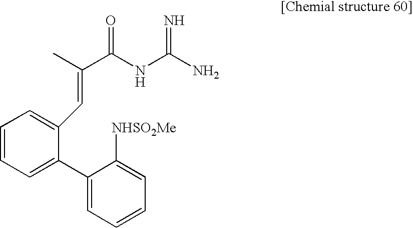 <Step 1>

Intermediate 1 and 2-methanesulfonamidephenyl boronic acid were reacted in the same manner as described in Example 1 to obtain the compound of Example 51.

MS: 373

### Example 52

### Synthesis of N-[(E)-3-(2′,3′-dimethoxy-biphenyl-2-yl)-2-methyl-acryloyl]-guanidine

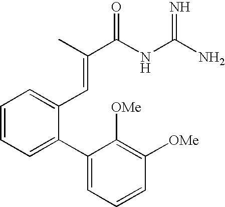 <Step 1>

Intermediate 1 and 2,3-dimethoxyphenyl boronic acid were reacted in the same manner as described in Example 1 to obtain the compound of Example 52.

MS: 340

### Example 53

### Synthesis of N-[(E)-3-(2′,4′-hydroxy-2-yl)-2-methyl-acryloyl]-guanidine

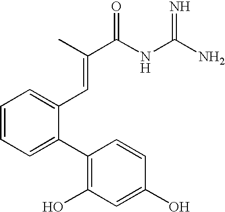 <Step 1>

Intermediate 1 (100 mg, 0.252 mmol) and 2,4-dimethoxyphenyl boronic acid (55.1 mg. 0.303 mmol) were dissolved in a mixed solution of dioxane and water (v/v=3/1, 2.0 mL). Pd(PPh₃)₄ (14.6 mg, 12.6 μmmol) and Na₂CO₃ (80.3 mg, 0.757 mmol) were added to the solution and then stirred at 90° C. for 2 hours. After cooling it to room temperature, the solvent was eliminated in vacuo and then purified by reversed phase HPLC (0.1% TFA in water/CH₃CN) to obtain the objective intermediate (107.2 mg, 94%).

MS: 340

<Step 2>

After 1.0 mol/L BBr₃ in dichloromethane solution was added to the intermediate obtained from Step 1 (100 mg, 0.221 mmol) at 0° C., the reaction temperature was elevated to 35° C. and then stirred for 6 hours. After cooling it to 0° C., it was diluted with dichloromethane and then saturated sodium hydrate carbonate solution was added to terminate the reaction. After adding saturated saline thereto, it was extracted with acetonitrile, the solvent was eliminated in vacuo and then purified by reversed phase HPLC (0.1% TFA in water/CH₃CN) to obtain the compound of Example 53 (79.8 mg, 85%).

MS: 312

### Example 54

### Synthesis of N-[(E)-3-(2′,3′-difluoro-4′-hydroxy-biphenyl-2-yl)-2-methyl-acryloyl]-guanidine

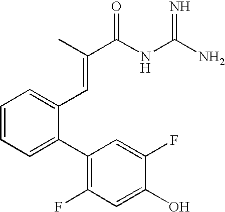 <Step 1>

4-bromo-2,5-difluoroanisole (223.0 mg, 1.00 mmol) and 2-formylphenyl boronic acid (179.9 mg. 1.20 mmol) were dissolved in a mixed solution of dioxane and water (v/v=3/1, 20 mL). Pd(PPh₃)₄ (57.8 mg, 0.05 mmol) and Na₂CO₃ (318.0 mg, 3.0 mmol) were added to the solution and then stirred at 90° C. for 2 hours. After cooling it to room temperature, dichloromethane was added thereto, washed with water and then dried over anhydrous MgSO₄. After the solvent was eliminated in vacuo, it was purified by silica gel column chromatography (Hexane/EtOAc) to obtain the objective aldehyde (200 mg, 81%).

MS: 249

<Step 2>

NaH (60% assay, 96.7 mg, 2.42 mmol) was suspended in THF (8 mL) and then cooled to 0° C. Triethyl-2-phosphonopropionate (576 mg, 2.42 mmol) in THF (2 mL) was added in a slow manner to the suspention. After stirring it for 15 minutes, the intermediate aldehyde obtained from Step 1 (200 mg, 0.806 mmol) in THF (2 mL) was added thereto and then stirred for 1 hour while gradually heating it to room temperature. EtOAc was added to the reaction solution and then washed with NaHCO₃ solution, water and saturated saline. After drying it over anhydrous MgSO₄, the solvent was eliminated in vacuo to obtain a crude product.

MS: 333

The resulting crude product was then dissolved in a mixed solution of THF and MeOH (v/v=5/3, 16 m). 2 N NaOH (5 mL, 10 mmol) was added to the solution and then stirred at 50° C. for 6 hours. After cooling it to 0° C., 2 N HCl was added to acidify the solution, dichloromethane was added, washed with water and saturated saline and then dried over anhydrous MgSO₄. After the solvent was eliminated in vacuo, it was purified by silica gel column chromatography (Hexane/EtOAc) to obtain the objective carboxylic acid (123 mg, 50%).

MS: 305

<Step 3>

The carboxylic acid obtained from Step 2 (123 mg, 0.383 mmol) was dissolved in DMF (5 mL), CDI (74.5 mg, 0.459 mmol) was added thereto and then stirred at room temperature for 30 minutes. N-Boc-guanidine (73.1 mg, 0.459 mmol) was added to the solution and then stirred for 19.5 hours. After the solvent was eliminated in vacuo, TFA (5 mL) was added to the residue and then stirred at 55° C. for 8 hours. After the solvent was eliminated in vacuo, it was purified by reversed phase HPLC (0.1% TFA in water/CH₃CN) to obtain the objective acylguanidine (17.3 mg, 10%).

MS: 346

<Step 4>

After 1.0 mol/L BBr₃ dichloromethane solution (3.0 mL, 3.0 mmol) was added to the acylguanidine obtained from Step 3 (10 mg, 0.0218 mmol) at 0° C., the reaction temperature was elevated to 35° C. and then stirred for 6 hours. After cooling it to 0° C., it was diluted with dichloromethane and then saturated sodium hydrogen carbonate solution was added thereto to terminate the reaction. After saturated saline was added thereto, it was extracted with acetonitrile, the solvent was eliminated in vacuo and then it was purified by reversed phase HPLC (0.1% TFA in water/CH₃CN) to obtain the compound of Example 54 (2.1 mg, 22%)

MS: 332

### Example 55

### Synthesis of (E)-2′-(3-guanidino-2-methyl-3-oxo-propenyl)-biphenyl-3-carboxylic acid hydroxyamide

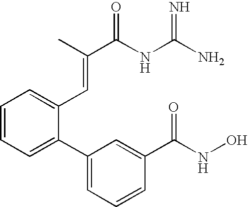 <Step 1>

3-bromo-benzoic acid (200 mg, 0.995 mmol) and tert-butoxyamine hydrochloride (111 mg, 0.887 mmol) were dissolved in dichloromethane (10 mL), triethylamine (0.34 mL, 2.42 mmol) and EDCI (186 mg, 0.967 mmol) were added to the solution at room temperature and then stirred for 13 hours. Then, dichloromethane was added thereto, washed with water, saturated NH₄Cl and saturated saline and then dried over anhydrous MgSO₄. After the solvent was eliminated in vacuo, it was purified by silica gel column chromatography (SiO₂, Hexame/EtOAc) to obtain the objective compound (147 mg, 54.2%).

MS: 273

<Step 2>

The compound obtained from Step 1 (147 mg, 0.539 mmol) and 2-formylphenyl boronic acid (147 mg, 0.539 mmol) were dissolved in a mixed solution of dioxane and water (v/v=3/1, 4.0 mL), Pd(PPh₃)₄ (31.2 mg, 27 μmol) and Na₂CO₃ (171 mg, 1.62 mmol) were added to the solution and then stirred at 90° C. for 3 hours. After cooling it to room temperature, the solvent was eliminated in vacuo, EtOAc was added thereto, the organic layer was washed with saturated NaHCO₃ solution and saturated saline and then dried over anhydrous MgSO₄. Then, the solvent was eliminated in vacuo to obtain a crude product (229 mg).

MS: 298

<Step 3>

NaH (60% assay, 97.2 mg, 2.43 mmol) was suspended in THF (6.0 mL) and then cooled to 0° C. Triethyl-2-phosphonopropionate (579 mg, 2.43 mmol) in THF (1.0 mL) was added dropwise in a slow manner to the suspention and then stirred for 15 minutes. Then, the crude product obtained from Step 2 in THF (1.0 mL) was added thereto in a slow manner and then stirred for 3 days while gradually heating it from 0° C. to room temperature. EtOAc was added to the reaction solution, washed with water and saturated saline, and then dried over anhydrous MgSO₄. Then, the solvent was eliminated in vacuo to obtain a residue.

The resulting residue was dissolved in THF (3.0 mL) and MeOH (1.0 mL), 2 N NaOH (1.5 mL, 3.0 mmol) was added thereto and then stirred at room temperature for 2 hours. The solvent was then eliminated in vacuo, 2 N HCl was added to acidify the solution and then the crystals precipitated were filtrated to obtain white crystals of a carboxylic acid which was a crude product (174 mg).

MS: 354

The carboxylic acid obtained from Step 3 (100 mg, 0.283 mmol) was dissolved in DMF (3.0 mL), CDI (55.1 mg, 0.340 mmol) was added thereto and then stirred at room temperature for 30 minutes. N-Boc-guanidine (53.3 mg, 0.340 mmol) was added to the solution and then stirred for 22 hours. After the solvent was eliminated in vacuo, TFA (4.0 mL) was added to the residue and then stirred for 17.5 hours. After concentrating the solvent in vacuo, it was purified by reversed phase HPLC (0.1% TFA in water/CH₃CN) to obtain the compound of Example 55 (28.5 mg, 22.3%)

MS: 339

### Example 56

### Synthesis of (E)-2′-(3-guanidino-2-methyl-3-oxo-propenyl)-biphenyl-4-carboxamidine

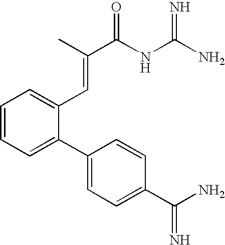 <Step 1>

Intermediate 1 (150 mg, 0.379 mmol) and 4-cyanophenyl boronic acid (111 mg. 0.758 mmol) were dissolved in a mixed solution of dioxane and water (v/v=4/1, 5.0 mL). Pd(PPh₃)₄ (21.9 mg, 19.0 μmol) and Na₂CO₃ (161 mg, 1.52 mmol) were added to the solution and then stirred at 90° C. for 2 hours. After cooling it to room temperature, the solvent was eliminated in vacuo and then purified by reversed phase HPLC (0.1% TFA in water/CH₃CN) to obtain the objective intermediate (84.3 mg, 53%).

MS: 305

<Step 2>

The intermediate obtained from Step 1 (70 mg, 0.167 mmol) was dissolved in EtOH (1.0 mL). 4 N HCl in dioxane (4.0 mL) was added to the solution and then stirred at room temperature for 48 hours. After the solvent was eliminated in vacuo, it was dissolved in EtOH (1.0 mL), (NH)₂CO₃ (161 mg, 1.67 mmol) was added thereto and then stirred at room temperature for 5 hours. After the solvent was eliminated in vacuo, it was purified by reversed phase HPLC (0.1% TFA in water/CH₃CN) to obtain the compound of Example 56 (38 mg, 52%)

MS: 322

### Example 57

### Synthesis of (E)-2′-(3-guanidino-2-methyl-3-oxo-propenyl)-2-methyl-biphenyl-4-carboxamidine

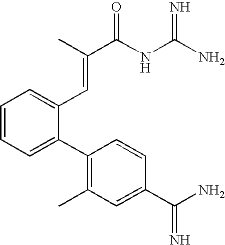 <Step 1>

Intermediate 1 (100 mg, 0.253 mmol) and 2-methyl-4-cyanophenyl boronic acid (61.0 mg. 0.379 mmol) were dissolved in a mixed solution of dioxane and water (v/v=3/1, 4.0 mL). Pd(PPh₃)₄ (14.6 mg, 13.0 μmol) and Na₂CO₃ (107 mg, 1.01 mmol) were added to the solution and then stirred at 80° C. for 12 hours. After cooling it to room temperature, the solvent was eliminated in vacuo and then purified by reversed phase HPLC (0.1% TFA in water/CH₃CN) to obtain the objective intermediate (90 mg, 80%).

MS: 319

<Step 2>

The intermediate obtained from Step 1 (90 mg, 0.208 mmol) was dissolved in EtOH (0.8 mL). 4 N HCl in dioxane (4.0 mL) was added to the solution and then stirred at room temperature for 72 hours. After the solvent was eliminated in vacuo, it was dissolved in EtOH (2.0 mL), (NH₄CO₃ (200 mg, 2.08 mmol) was added thereto and then stirred at room temperature for 5 hours. After the solvent was eliminated in vacuo, it was purified by reversed phase HPLC (0.1% TFA in water/CH₃CN) to obtain the compound of Example 57 (70 mg, 60%)

MS: 336

### Example 58

### Synthesis of (E)-3-fluoro-2′-(3-guanidino-2-methyl-3-oxo-propenyl)-biphenyl-4-carboxamidine

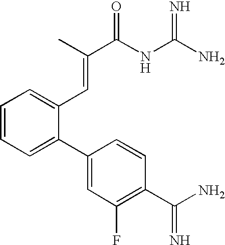 <Step 1>

Intermediate 1 (100 mg, 0.253 mmol) and 3-fluoro-4-cyanophenyl boronic acid (62.5 mg, 0.379 mmol) were dissolved in a mixed solution of dioxane and water (v/v=3/1, 4.0 mL). Pd(PPh₃)₄ (14.6 mg, 13.0 μmol) and Na₂CO₃ (107 mg, 1.01 mmol) were added to the solution and then stirred at 80° C. for 12 hours. After cooling it to room temperature, the solvent was eliminated in vacuo and then purified by reversed phase HPLC (0.1% TFA in water/CH₃CN) to obtain the objective intermediate (84 mg, 76%).

MS: 323

<Step 2>

The intermediate obtained from Step 1 (84 mg, 0.192 mmol) was dissolved in EtOH (0.6 mL). 4 N HCl in dioxane (3.0 mL) was added to the solution and then stirred at room temperature for 6 days. After the solvent was eliminated in vacuo, it was dissolved in EtOH (2.0 mL), (NH₄)₂CO₃ (200 mg, 2.08 mmol) was added thereto and then stirred at room temperature for 12 hours. After the solvent was eliminated in vacuo, it was purified by reversed phase HPLC (0.1% TFA in water/CH₃CN) to obtain the compound of Example 58 (38 mg, 35%)

MS: 340

### Example 59

### Synthesis of N-[(E)-3-(2′-fluoro-4′-hydroxy-biphenyl-2-yl)-2-methyl-acryloyl]-guanidine

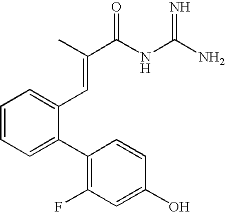 <Step 1>

Intermediate 1 (50 mg, 0.126 mmol) and 2-fluoro-4-methoxyphenyl boronic acid (23.6 mg, 0.138 mmol) were dissolved in a mixed solution of dioxane and water (v/v=3/1, 2.0 mL). Pd(PPh₃)₄ (7.29 mg, 6.30 μmol) and Na₂CO₃ (40.0 mg, 0.378 mmol) were added to the solution and then stirred at 90° C. for 2.5 hours. After cooling it to room temperature, the solvent was eliminated in vacuo and then purified by reversed phase HPLC (0.1% TFA in water/CH₃CN) to obtain the objective coupling product (38.6 mg, 69.5%).

MS: 328

<Step 2>

The coupling product obtained from Step 1 (25 mg, 0.0567 mmol) was dissolved in CH₂Cl₂ (1.0 mL), 1.0 mol/L BBr₃ dichloromethane solution (0.42 mL, 0.420 mmol) was added to the solution and then stirred at room temperature for 4.5 hours. After concentrating the solvent in vacuo, it was purified by reversed phase HPLC (0.1% TFA in water/CH₃CN) to obtain the compound of Example 59 (22.3 mg, 92.1%).

1H-NMR (d-DMSO, 400 MHz) σ 2.00 (s, 3H), 6.62 (dd, 1H, J=2.6, 12 Hz), 6.67 (dd, 1H, J=2.6, 8.2 Hz), 7.07 (t, 1H, J=8.8 Hz), 7.26 (s, 1H), 7.33-7.41 (m, 1H), 7.43-7.53 (m, 3H), 8.24 (bs, 4H), 10.6 (bs, 1H)

MS: 314

### Example 60

### Synthesis of N-[(E)-3-(2′-chloro-4′-hydroxy-biphenyl-2-yl)-2-methyl-acryloyl]-guanidine

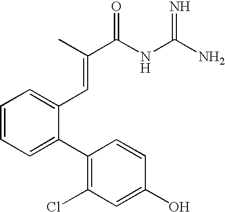 <Step 1>

Intermediate 1 (50 mg, 0.126 mmol) and 2-chloro-4-methoxyphenyl boronic acid (25.9 mg, 0.139 mmol) were dissolved in a mixed solution of dioxane and water (v/v=3/1, 2.0 mL). Pd(PPh₃)₄ (8.04 mg, 6.95 μmol) and Na₂CO₃ (41.6 mg, 0.378 mmol) were added to the solution and then stirred at 90° C. for 2 hours. After cooling it to room temperature, the solvent was eliminated in vacuo and then purified by reversed phase HPLC (0.1% TFA in water/CH₃CN) to obtain the objective coupling product (38.9 mg, 67.6%).

MS: 345

<Step 2>

The coupling product obtained from Step 1 (33 mg, 0.0722 mmol) was dissolved in CH₂Cl₂ (1.0 mL), 1.0 mol/L BBr₃ dichloromethane solution (0.50 mL, 0.50 mmol) was added to the solution and then stirred at room temperature for 2 hours. After the solvent was eliminated in vacuo, it was purified by reversed phase HPLC (0.1% TFA in water/CH₃CN) to obtain the compound of Example 60 (22.4 mg, 69.9%).

1H-NMR (d-DMSO, 400 MHz) σ 1.96 (d, 3H, J=1.3 Hz), 6.80 (dd, 1H, J=2.5, 8.5 Hz), 6.91 (d, 1H, J=2.5 Hz), 7.07 (d, 1H, J=8.5 Hz), 7.15-7.18 (m, 1H), 7.28-7.33 (m, 1H), 7.44-7.51 (m, 2H), 8.26 (bs, 4H), 10.1 (s, 1H)

MS: 331

### Example 61

### Synthesis of N-[(E)-3-(4′-hydroxy-3′-methyl-biphenyl-2-yl)-2-methyl-acryloyl]-guanidine

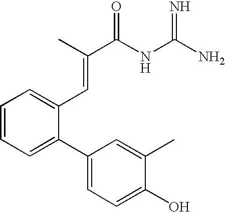 <Step 1>

Intermediate 1 (50 mg, 0.126 mmol) and 3-methyl-4-methoxyphenyl boronic acid (23.9 mg, 0.139 mmol) were dissolved in a mixed solution of dioxane and water (v/v=3/1, 2.0 mL). Pd(PPh₃)₄ (6.94 mg, 6.00 μmol) and Na₂CO₃ (41.6 mg, 0.378 mmol) were added to the solution and then stirred at 90° C. for 2 hours. After cooling it to room temperature, the solvent was eliminated in vacuo and then purified by reversed phase HPLC (0.1% TFA in water/CH₃CN) to obtain the objective coupling product (40.5 mg, 73.5%).

MS: 324

<Step 2>

The coupling product obtained from Step 1 (30 mg, 0.0671 mmol) was dissolved in CH₂Cl₂ (1.0 mL), 1.0 mol/L BBr₃ dichloromethane solution (0.50 mL, 0.50 mmol) was added to the solution and then stirred at room temperature for 5 hours. After the solvent was eliminated in vacuo, it was purified by reversed phase HPLC (0.1% TFA in water/CH₃CN) to obtain the compound of Example 61 (27.2 mg, 95.8%).

1H-NMR (d-DMSO, 400 MHz) σ 2.00 (d, 3H, J=1.2 Hz), 6.81 (d, 1H, J=8.3 Hz), 6.92 (dd, 1H, J=2.2, 8.4 Hz), 7.06 (d, 1H, J=1.7 Hz), 7.34 (d, 1, J=1.2), 7.35-7.38 (m, 4H), 8.22-8.52 (m, 4H), 9.53 (s, 1H)

MS: 310

### Example 62

### Synthesis of N-[(E)-3-(3′-fluoro-4′-hydroxy-5-methylbiphenyl-2-yl)-2-methyl-acryloyl]-guanidine

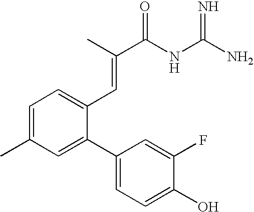 <Step 1>

Intermediate 2 (50 mg, 0.122 mmol) and 3-fluoro-4-hydroxyphenyl boronic acid (22.8 mg, 0.146 mmol) were dissolved in a mixed solution of dioxane and water (v/v=3/1, 2.0 mL). Pd(PPh₃)₄ (7.0 mg, 6.1 μmol) and Na₂CO₃ (38.8 mg, 0.366 mmol) were added to the solution and then stirred at 90° C. for 2 hours. After cooling it to room temperature, the solvent was eliminated in vacuo and then purified by reversed phase HPLC (0.1% TFA in water/CH₃CN) to obtain the compound of Example 62 (10.9 mg, 20%).

MS: 328

### Example 63

### Synthesis of N-[(E)-3-(4′-fluoro-3′-hydroxy-5-methyl-biphenyl-2-yl)-2-methyl-acryloyl]-guanidine

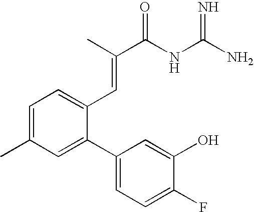 <Step 1>

Intermediate 2 (50 mg, 0.122 mmol) and 4-fluoro-3-hydroxyphenyl boronic acid (22.8 mg, 0.146 mmol) were dissolved in a mixed solution of dioxane and water (v/v=3/1, 2.0 mL). Pd(PPh₃)₄ (7.0 mg, 6.10 μmol) and Na₂CO₃ (38.8 mg, 0.366 mmol) were added to the solution and then stirred at 90° C. for 2 hours. After cooling it to room temperature, the solvent was eliminated in vacuo and then purified by reversed phase HPLC (0.1% TFA in water/CH₃CN) to obtain the compound of Example 63 (7.9 mg, 15%).

MS: 328

### Example 64

### Synthesis of 4-[(E)-6-(3-guanidino-2-methyl-3-oxo-propenyl)-3′-hydroxy-biphenyl-3-yloxy]-benzenesulfonamide

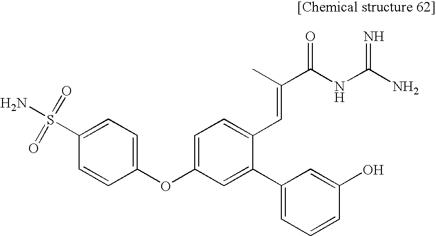 <Step 1>

The intermediate obtained from Step 3 in Example 65 and 3-hydroxyphenyl boronic acid were reacted to obtain the compound of Example 64 in the same manner as described in Example 65.

MS: 477

### Example 65

### Synthesis of 4-[(E)-6-(3-guanidino-2-methyl-3-oxo-propenyl)-4′-hydroxy-biphenyl-3-yloxy]-benzenesulfonamide

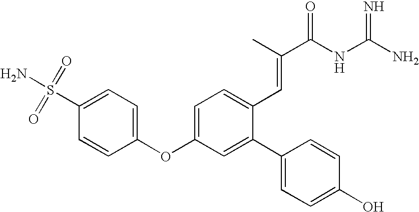 <Step 1>

2-bromo-4-fluorobenzaldehyde (500 mg, 2.46 mmol) was dissolved in DMF (50 mL), 4-hydroxybenzenesulfonamide (511 mg, 2.95 mmol) and K₂CO₃ (408 mg, 2.96 mmol) were added thereto and then stirred at 100° C. for 2 hours. After cooling it to room temperature, EtOAc was added thereto, washed with water and saturated saline and then dried over anhydrous MgSO₄. After the solvent was eliminated in vacuo, it was purified by silica gel chromatography (Hexane/EtOc) to obtain the objective ether (690 mg, 78%).

<Step 2>

NaH (60% assay, 116 mg, 2.9 mmol) was suspended in DMF (50 mL) and then triethyl 2-phosphonopropionate (0.611 mg, 2.9 mmol) was added dropwise in a slow manner to the solution and then stirred for 15 minutes. Then, the compound obtained from Step 1 in DMF (3 mL) was added thereto in a slow manner and then stirred for 18 hours. EtOAc was then added to the reaction solution, washed with water and saturated saline and then dried over anhydrous MgSO₄. After the solvent was eliminated in vacuo, it was purified by silica gel chromatography (Hexane/EtOAc) to obtain the objective ester (880 mg, 70%). The resulting compound was then dissolved in THF (5 mL) and MeOH (2 mL), 1 N NaOH (8 mL, 8 mmol) was added thereto and then stirred at room temperature for 8 hours. After the solvent was eliminated in vacuo, 2 N HCl was added to acidify the solution and then crystals precipitated were filtrated to obtain the objective carboxylic acid (830 mg, 100%).

<Step 3>

The carboxylic acid obtained from Step 2 (830 mg, 2.0 mmol) was dissolved in DMF (10 mL), CDI (375 mg, 2.3 mmol) was added thereto and then stirred at room temperature for 30 minutes. N-Boc-guanidine (453 mg, 2.85 mmol) was added to the solution and then stirred for 16 hours. Then, EtOAc was added thereto, washed with water and saturated saline and then dried over anhydrous MgSO₄. TFA (10 mL) was added to the residue and then stirred for 1.5 hours. After concentrating the solvent in vacuo, it was purified by reversed phase HPLC (0.1% TFA in water/CH₃CN) to obtain an intermediate acylguanidine (410 mg, 35%).

MS: 454

<Step 4>

The compound of Example 65 was obtained from the intermediate obtained from Step 3 in the same manner as described in Example 2.

MS: 477

### Example 66

### Synthesis of N-[(E)-3-(4′-hydroxy-3′-methoxy-5-methyl-biphenyl-2-yl)-2-methyl-acryloyl]-guanidine

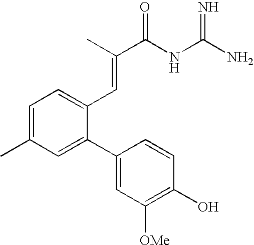 <Step 1>

Intermediate 2 (50 mg, 0.122 mmol) and 2-methoxy-4-(4,4,5,5-tetramethyl-1,3,2-dioxaborolane-2-yl)phenol (36.6 mg, 0.0146 mmol) were dissolved in a mixed solution of dioxane ad water (v/v=3/1, 2.0 mL). Pd(PPh₃)₄ (7.00 mg, 6.10 μmol) and Na₂CO₃ (38.8 mg, 0.366 mmol) were added to the solution and then stirred at 90° C. for 2.5 hours. After cooling it to room temperature, the solvent was eliminated in vacuo and then purified by reversed phase HPLC (0.1% TFA in water/CH₃CN) to obtain the compound of Example 66 (1.7 mg, 3%).

MS: 388

### Example 67

### Synthesis of N-[(E)-3-(4′-hydroxy-6-methyl-biphenyl-2-yl)-2-methyl-acryloyl]-guanidine

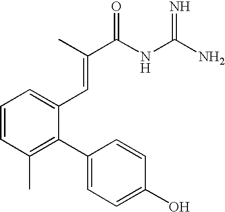 <Step 1>

2-methyl-3-bromobenzoic acid (1 g, 4.65 mmol) and triethylamine (0.97 mL, 6.78 mmol) were dissolved in THF (20 mL), chloroformic acid ethyl (0.49 mL, 6.11 mmol) was added thereto while cooling it by ice and then stirred for 15 minutes. The precipitate was then eliminated by suction filtration, 1 g of ice and sodium borohydride (260 mg, 6.78 mmol) were added to the resulting filtrate while cooling it by ice and then stirred overnight. It was washed with water and saturated saline and then dried over anhydrous MgSO₄. The solvent was then eliminated in vacuo to obtain a residue. The resulting residue was dissolved in chloroform (50 mL), manganese dioxide (2 g, 22.5 mmol) was added thereto and then stirred overnight. After filtration, the solvent was eliminated in vacuo and then purified by reversed phase HPLC (0.1% TFA in water/CH₃CN) to obtain an aldehyde (640 mg, 69%).

MS: 199

<Step 2>

NaH (60% assay, 193 mg, 4.82 mmol) was suspended in DMF (10 mL) and then cooled to 0° C. Triethyl 2-phosphonopropionate (1.05 mg, 4.82 mmol) in DMF (10 mL) was added dropwise in a slow manner to the solution and then stirred for 15 minutes. Then, the aldehyde obtained from Step 1 (640 mg, 3.22 mmol) in DMF (3 mL) was added thereto in a slow manner and then stirred for 18 hours while gradually heating it from 0° C. to room temperature. EtOAc was then added to the reaction solution, washed with water and saturated saline and then dried over anhydrous MgSO₄. The solvent was eliminated in vacuo to obtain a residue. The resulting residue was then dissolved in THF (10 mL) and MeOH (4 mL), 2 N NaOH (8 mL, 8 mmol) was added thereto and then stirred at room temperature for 8 hours. The solvent was then eliminated in vacuo, 2 N HCl was added to acidify the solution and then the crystals precipitated were filtrated to obtain white crystals of the objective carboxylic acid (600 mg, 93%).

MS: 256

<Step 3>

The carboxylic acid obtained from Step 2 (600 mg, 3.0 mmol) was dissolved in DMF (10 mL), CDI (610 mg, 3.8 mmol) was added thereto and then stirred at room temperature for 30 minutes. N-Boc-guanidine (716 mg, 4.5 mmol) was added to the solution and then stirred for 16 hours. After the solvent was eliminated in vacuo, TFA (10 mL) was added to the residue and then stirred for 1.5 hours. After concentrating the solvent in vacuo, it was purified by reversed phase HPLC (0.1% TFA in water/CH₃CN) to obtain the objective acylguanidine (250 mg, 20%)

MS: 297

<Step 4>

The acylguanidine obtained from Step 3 (50 mg, 0.122 mmol) and 4-hydroxyphenyl boronic acid (18.5 mg, 0.134 mmol) were dissolved in a mixed solution of dioxane and water (v/v=3/1, 4.0 mL). Pd(PPh₃)₄ (7.06 mg, 6.10 μmol) and Na₂CO₃ (40.3 mg, 0.366 mmol) were added to the solution and then stirred at 90° C. for 14 hours. After cooling it to room temperature, the solvent was eliminated in vacuo and then purified by reversed phase HPLC (0.1% TFA in water/CH₃CN) to obtain the compound of Example 67 (20.0 mg, 38.8%).

MS: 310

### Example 68

### Synthesis of N-[(E)-3-(3′-hydroxy-6-methyl-biphenyl-2-yl)-2-methyl-acryloyl]-guanidine

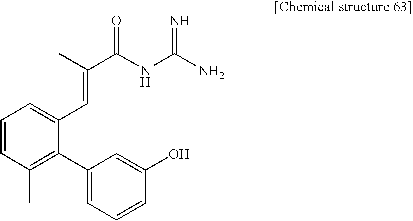 <Step 1>

The intermediate obtained from Step 3 in Example 67 (50 mg, 0.122 mmol) and 3-hydroxyphenyl boronic acid (18.5 mg, 0.134 mmol) were dissolved in a mixed solution of dioxane and water (v/v=3/1, 2.0 mL). Pd(PPh₃)₄ (7.06 mg, 4.56 μmol) and Na₂CO₃ (40.3 mg, 0.366 mmol) were added to the solution and then stirred at 90° C. overnight. After cooling it to room temperature, the solvent was eliminated in vacuo and then purified by reversed phase HPLC (0.1% TFA in water/CH₃CN) to obtain the compound of Example 68 (20.9 mg, 40.5%).

MS: 310

### Example 69

### Synthesis of N-[(E)-3-(3′,4′-dihydroxy-5-methyl-biphenyl-2-yl)-2-methyl-acryloyl]-guanidine

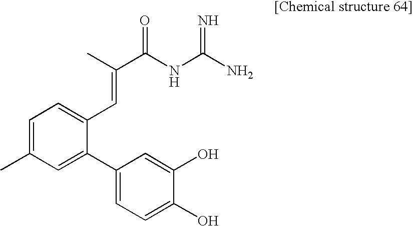 <Step 1>

Intermediate 2 and 2,3-dihydroxyphenyl boronic acid were reacted in the same manner as described in Example 1 to obtain the compound of Example 69.

MS: 326

### Example 70

### Synthesis of N-[(E)-3-(3-bromo-4′-hydroxy-biphenyl-2-yl)-2-methyl-acryloyl]-guanidine

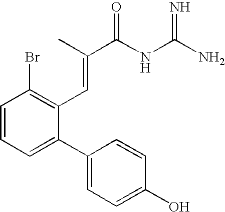 <Step 1>

Intermediate 3 (100 mg, 0.210 mmol) and 4-hydroxyphenyl boronic acid (29.1 mg, 0.210 mmol) were dissolved in a mixed solution of dioxane and water (v/v=4/1, 2.5 mL). PdCl₂ (dppf) (8.5 mg, 11.0 μmol) and Na₂CO₃ (89.0 mg, 0.840 mmol) were added to the solution and then stirred at 90° C. for 12 hours. After cooling it to room temperature, the solvent was eliminated in vacuo and purified by reversed phase HPLC (0.1% TFA in water/CH₃CN) to obtain the compound of Example 70 (85.0 mg).

MS: 376

### Example 71

### Synthesis of N-{(E)-3-[4′-hydroxy-5-(4-hydroxy-phenoxy)-biphenyl-2-yl]-2-methyl-acryloyl}-guanidine

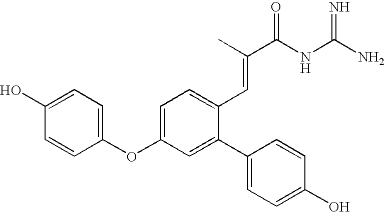 <Step 1>

4-methoxy-phenol (283 mg, 2.28 mmol) and 2-bromo-4-fluorobenzaldehyde (386 mg, 1.90 mmol) were dissolved in DMF (10 mL), K₂CO₃ (315 mg, 2.28 mmol) was added to the solution and then stirred at 100° C. for 2 hours. Then, the reaction solution was cooled, EtOAc was added thereto, washed with water and saturated saline and then dried over anhydrous MgSO₄. The solvent was then eliminated in vacuo to obtain a residue. NaH (60% assay, 114 mg, 2.85 mmol) was suspended in DMF (10 mL) and then cooled to 0° C. Triethyl 2-phosphonopropionate (0.62 mL, 2.85 mmol) in DMF (10 mL) was added dropwise in a slow manner to the solution and then stirred for 15 minutes. Then, the resulting residue in DMF (3 mL) was added in a slow manner and then stirred for 18 hours while gradually heating it from 0° C. to room temperature. EtOAc was then added to the reaction solution, washed with water and saturated saline and then dried over anhydrous MgSO₄. The residue obtained by eliminating the solvent in vacuo was then purified by silica gel column chromatography to obtain an ester (230 mg, 30%).

MS: 392

The resulting ester (230 mg, 0.59 mmol) was dissolved in THF (5 mL) and MeOH (2 mL), 1 N NaOH (4 mL, 4 mmol) was added thereto and then stirred at room temperature for 8 hours. The solvent was then eliminated in vacuo, 2N HCl was added to acidify the solution and then the crystals precipitated were filtrated to obtain white crystals of the objective carboxylic acid (210 mg, 98%).

MS: 364

<Step 2>

The carboxylic acid obtained from Step 1 (210 mg, 0.58 mmol) was dissolved in DMF (10 mL), CDI (113 mg, 0.70 mmol) was added thereto and then stirred at room temperature for 30 minutes. N-Boc-guanidine (138 mg, 0.87 mmol) was added to the solution and then stirred for 16 hours. After the solvent was eliminated in vacuo, TFA (10 mL) was added to the residue at 0° C. and then stirred for 1.5 hours. After concentrating the solvent in vacuo, it was purified by reversed phase HPLC (0.1% TFA in water/CH₃CN) to obtain the objective acylguanidine (120 mg, 52%).

MS: 282

<Step 3>

The acylguanidine obtained from Step 2 (50 mg, 0.096 mmol) and 4-hydroxyphenyl boronic acid (14.6 mg, 0.106 mmol) were dissolved in a mixed solution of dioxane and water (v/v=3/1, 4.0 mL). Then, Pd(PPh₃)₄ (5.55 mg, 4.80 μmol) and Na₂CO₃ (30.5 mg, 0.288 mmol) were added to the solution and then stirred at 90° C. for 5 hours. After cooling it to room temperature, the solvent was eliminated in vacuo and then purified by reversed phase HPLC (0.1% TFA in water/CH₃CN) to obtain the coupling product (34.1 mg, 66.8%).

1H-NMR (d-DMSO, 400 MHz) σ 2.03 (s, 3H), 3.76 (s, 3H), 6.80 (d, 2H, J=8.6 Hz), 6.89 (d, 1H, J=2.7 Hz), 6.94 (dd, 1H, J=2.7, 8.6 Hz), 7.00 (d, 2H, J=9.0 Hz), 7.08 (d, 2H, J=8.6 Hz), 7.12 (d, 2H, J=9.0 Hz), 7.27 (s, 1H), 7.45 (d, 1H, J=8.6 Hz), 8.24 (bs, 4H), 9.67 (s, 1H)

MS: 418

<Step 4>

The coupling product obtained from Step 3 (30 mg, 0.0564 mmol) was dissolved in CH₂Cl₂ (1.0 mL), 1.0 mol/L BBr₃ dichloromethane solution (0.50 mL, 0.50 mmol) was added thereto and then stirred at room temperature for 3 hours. After the solvent was eliminated in vacuo, it was purified by reversed phase HPLC (0.1% TFA in water/CH₃CN) to obtain the compound of Example 71 (22.5 mg, 77.1%).

1H-NMR (d-DMSO, 400 MHz) σ 2.02 (d, 3H, J=1.2 Hz), 6.79 (d, 2H, J=8.6 Hz), 6.81 (d, 2H, J=9.0 Hz), 6.86 (d, 1H, J=2.7 Hz), 6.91 (dd, 1H, J=2.7, 8.6 Hz), 6.98 (d, 2H, J=9.0 Hz), 7.09 (d, 2H, J=8.6 Hz), 7.22 (s, 1H), 7.43 (d, 1H, J=8.6 Hz), 8.33 (bs, 4H), 9.45 (s, 1H), 9.68 (s, 1H)

MS: 404

### Example 72

### Synthesis of N-[(E)-3-(2,4-di(4-hydroxyphenyl)-phenyl)-2-methyl-acryloyl]-guanidine

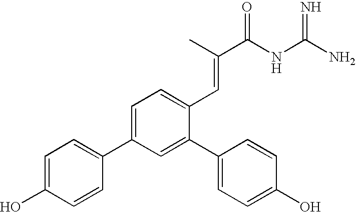 <Step 1>

Intermediate 4 (31 mg, 0.0653 mmol) and 4-hydroxyphenyl boronic acid (21.6 mg, 0.157 mmol) were dissolved in a mixed solution of dioxane and water (v/v=3/1, 2.0 mL). Pd(PPh₃)₄ (7.5 mg, 6.53 μmol) and Na₂CO₃ (41.5 mg, 0.392 mmol) were added to the solution and then stirred at 90° C. for 2 hours. After cooling it to room temperature, the solvent was eliminated in vacuo and then purified by reversed phase HPLC (0.1% TFA in water/CH₃CN) to obtain the compound of Example 72 (14.1 mg, 43%).

MS: 388

### Example 73

### Synthesis of N-[(E)-3-(4′-hydroxy-5-methoxy-biphenyl-2-yl)-2-methyl-acryloyl]-guanidine

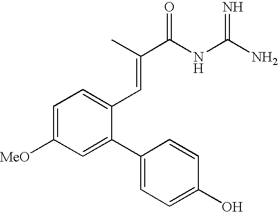 <Step 1>

NaOMe in MeOH (0.46 mL, 2.28 mmol) was dissolved in DMF (10 mL), K₂CO₃ (315 mg, 2.28 mmol) was added thereto and stirred for 15 minutes. 2-bromo-4-fluorobenzaldehyde (386 mg, 1.9 mmol) was added to the solution and then stirred at 100° C. for 2 hours. After cooling it to room temperature, EtOAc was added thereto, washed with NaHCO₃ solution and saturated saline and then dried over anhydrous MgSO₄. Then, the solvent was eliminated in vacuo to obtain the objective aldehyde.

MS: 216

<Step 2>

NaH (60% assay, 114 mg, 2.85 mmol) was suspended in DMF (10 mL) and then cooled to 0° C. Triethyl-2-phosphonopropionate (0.62 mL, 2.85 mmol) in DMF (5 mL) was added dropwise in a slow manner to the solution and then stirred for 15 minutes. Then, the aldehyde obtained from Step 1 in DMF (3 mL) was added thereto in a slow manner and then stirred for 18 hours while gradually heating it from 0° C. to room temperature. EtOAc was added to the reaction solution, washed with water and saturated saline and then dried over anhydrous MgSO₄. Then, the solvent was eliminated in vacuo to obtain a residue. The resulting residue was dissolved in THF (5 mL) and MeOH (2 mL), 1 N NaOH (4 mL, 4 mmol) was added thereto and then stirred at room temperature for 8 hours. Then, the solvent was eliminated in vacuo, 2 N HCl was added to acidify the solution and then the crystals precipitated were filtrated to obtain white crystals of the objective carboxylic acid (90 mg, 17%).

MS: 272

<Step 3>

The carboxylic acid obtained from Step 2 (90 mg, 0.33 mmol) was dissolved in DMF (4 mL), CDI (75 mg, 0.45 mmol) was added thereto and then stirred at room temperature for 30 minutes. N-Boc-guanidine (73 mg, 0.45 mmol) was added to the solution and then stirred for 16 hours. After the solvent was eliminated in vacuo, TFA (10 mL) was added to the residue and then stirred for 1.5 hours. After concentrating the solvent in vacuo, it was purified by reversed phase HPLC (0.1% TFA in water/CH₃CN) to obtain the objective acylguanidine (80 mg, 57.0%).

MS: 313

<Step 4>

The acylguanidine obtained from Step 3 (33.8 mg, 0.079 mmol) and 4-hydroxyphenyl boronic acid (36.0 mg, 0.261 mmol) were dissolved in a mixed solution of dioxane and water (v/v=3/1, 2.0 mL). Pd(PPh₃)₄ (13.5 mg, 11.7 μmol) and Na₂CO₃ (75.3 mg, 0.711 mmol) were added to the solution and then stirred at 90° C. for 6 hours. After cooling it to room temperature, the solvent was eliminated in vacuo and then purified by reversed phase HPLC (0.1% TFA in water/CH₃CN) to obtain the compound of Example 73 (11.0 mg, 31.7%).

1H-NMR (d-DMSO, 400 MHz) σ 2.04 (d, 3H, J=1.2 Hz), 3.84 (s, 3H), 6.79-6.86 (m, 3H), 7.00 (dd, 2H, J=2.7, 8.6 Hz), 7.15 (d, 2H, J=8.6 Hz), 7.27 (s, 1H), 7.42 (d, 1H, J=8.6 Hz), 8.38 (bs, 4H), 9.67 (s, 1H)

MS: 326

### Example 74

### Synthesis of N-[(E)-3-(3′-hydroxy-4′-methoxy-5-methyl-biphenyl-2-yl)-2-methyl-acryloyl]-guanidine

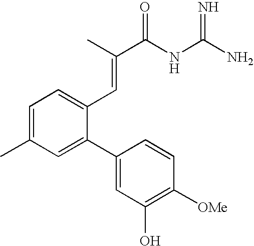 <Step 1>

Intermediate 2 (100 mg, 0.244 mmol) and 3-hydroxy-4-methoxyphenyl boronic acid pinacol ester (73.2 mg, 0.293 mmol) were dissolved in a mixed solution of dioxane and water (v/v=3/1, 4.0 mL). Pd(PPh₃)₄ (14.1 mg, 0.0122 mmol) and Na₂CO₃ (155.1 mg, 1.463 mmol) were added to the solution and then stirred at 90° C. for 2 hours. After cooling it to room temperature, the solvent was eliminated in vacuo and then purified by reversed phase HPLC (0.1% TFA in water/CH₃CN) to obtain the compound of Example 74 (11.6 mg, 10%).

MS: 340

### Example 75

### Synthesis of N-[(E)-3-(2,4-di(3,5-dimethyl-4-hydroxyphenyl)-phenyl)-2-methyl-acryloyl]-guanidine

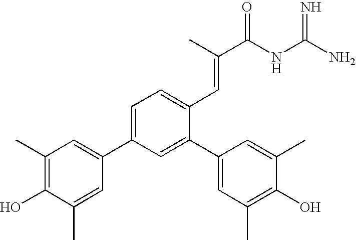 <Step 1>

Intermediate 4 (50 mg, 0.105 mmol) and 2,6-dimethyl-4-(4,4,5,5-tetramethyl-1,3,2-dioxaborolane-2-yl)-phenol (62.7 mg, 0.253 mmol) were dissolved in a mixed solution of dioxane and water (v/v=3/1, 2.0 mL). Pd(PPh₃)₄ (12.2 mg, 0.0105 mmol) and Na₂CO₃ (133.8 mg, 1.262 mmol) were added to the solution and then stirred at 90° C. for 2 hours. After cooling it to room temperature, the solvent was eliminated in vacuo and then purified by reversed phase HPLC (0.1% TFA in water/CH₃CN) to obtain the compound of Example 75 (7.9 mg, 13%).

MS: 444

### Example 76

### Synthesis of N-[(E)-3-(2,4-di(3-hydroxy-4-fluorophenyl)-phenyl)-2-methyl-acryloyl]-guanidine

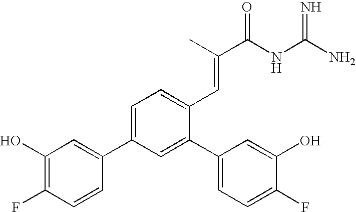 <Step 1>

Intermediate 4 (50 mg, 0.105 mmol) and 4-fluoro-3-hydroxyphenyl boronic acid (39.4 mg, 0.253 mmol) were dissolved in a mixed solution of dioxane and water (v/v=3/1, 2.0 mL). Pd(PPh₃)₄ (12.2 mg, 0.0105 mmol) and Na₂CO₃ (133.8 mg, 1.262 mmol) were added to the solution and then stirred at 90° C. for 2 hours. After cooling it to room temperature, the solvent was eliminated in vacuo and then purified by reversed phase HPLC (0.1% TFA in water/CH₃CN) to obtain the compound of Example 76 (26.4 mg, 47%).

MS: 424

### Example 77

### Synthesis of N-[(E)-3-(2,4-di(3-hydroxyphenyl)-phenyl)-2-methyl-acryloyl]-guanidine

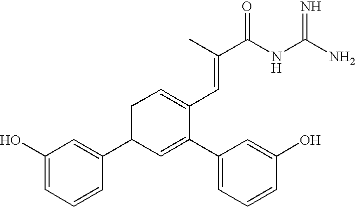 <Step 1>

Intermediate 4 (50 mg, 0.105 mmol) and 3-hydroxyphenyl boronic acid (34.8 mg, 0.253 mmol) were dissolved in a mixed solution of dioxane and water (v/v=3/1, 2.0 mL). Pd(PPh₃)₄ (12.2 mg, 0.0105 mmol) and Na₂CO₃ (133.8 mg, 1.262 mmol) were added to the solution and then stirred at 90° C. for 2 hours. After cooling it to room temperature, the solvent was eliminated in vacuo and then purified by reversed phase HPLC (0.1% TFA in water/CH₃CN) to obtain the compound of Example 77 (20.9 mg, 40%).

MS: 388

### Example 78

### Synthesis of N-[(E)-3-(2,4-di(3-methoxy-4-hydroxyphenyl)-phenyl)-2-methyl-acryloyl]-guanidine

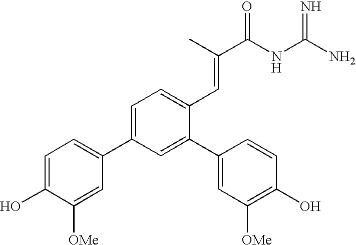 <Step 1>

Intermediate 4 (50 mg, 0.105 mmol) and 2-methoxy-4-(4,4,5,5-tetramethyl-1,3,2-dioxaborolane-2-yl) phenol (63.2 mg, 0.253 mmol) were dissolved in a mixed solution of dioxane and water (v/v=3/1, 2.0 mL). PdCl₂ (dppf) CH₂Cl₂ (8.6 mg, 0.0105 mmol) and Na₂CO₃ (133.8 mg, 1.262 mmol) were added to the solution and then stirred at 90° C. for 2 hours. After cooling it to room temperature, the solvent was eliminated in vacuo and then purified by reversed phase HPLC (0.1% TFA in water/CH₃CN) to obtain the compound of Example 78 (2.1 mg, 4%).

MS: 448

### Example 79

### Synthesis of (E)-3-fluoro-2′-(3-guanidino-2-methyl-3-oxo-propenyl)-5′-methyl-biphenyl-4-carboxamidine

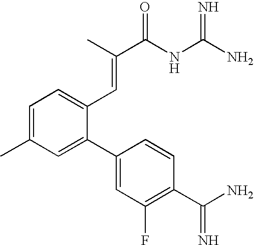 <Step 1>

Intermediate 2 (100 mg, 0.244 mmol) and 3-fluoro-4-cyanophenyl boronic acid (60.3 mg, 0.366 mmol) were dissolved in a mixed solution of dioxane and water (v/v=3/1, 4.0 mL). Pd(PPh₃)₄ (14.1 mg, 12.0 μmol) and Na₂CO₃ (103.5 mg, 0.976 mmol) were added to the solution and then stirred at 80° C. for 6 hours. After cooling it to room temperature, the solvent was eliminated in vacuo and then purified by reversed phase HPLC (0.1% TFA in water/CH₃CN) to obtain the objective intermediate (80 mg, 73%).

MS: 337

<Step 2>

The intermediate obtained from Step 1 (60 mg, 0.133 mmol) was dissolved in EtOH (0.4 mL). 4 N HCl in dioxane (2.0 mL) was added to the solution and stirred at room temperature for 36 hours. After the solvent was eliminated in vacuo, it was dissolved in EtOH (2.0 mL), (NH₄)₂CO₃ (200 mg, 2.08 mmol) was added thereto and then stirred at room temperature for 5 hours. After the solvent was eliminated in vacuo, it was purified by reversed phase HPLC (0.1% TFA in water/CH₃CN) to obtain the compound of Example 79 (7.0 mg, 11%).

MS: 355

### Example 80

### Synthesis of N-[(E)-3-(4,4″-dihydroxy-[1,1′;3′,1″]terphenyl-2′-yl)-2-methyl-acryloyl]-guanidine

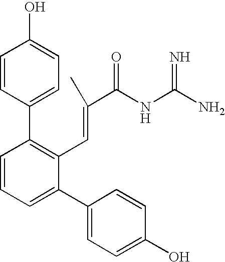 <Step 1>

Intermediate 3 (20 mg, 0.042 mmol) and 4-hydroxyphenyl boronic acid (14.6 mg, 0.106 mmol) were dissolved in a mixed solution of dioxane and water (v/v=3/1, 4.0 mL). Pd(PPh₃)₄ (5.55 mg, 4.80 μmol) and Na₂CO₃ (30.5 mg, 0.288 mmol) were added to the solution and then stirred at 90° C. for 5 hours. After cooling it to room temperature, the solvent was eliminated in vacuo and then purified by reversed phase HPLC (0.1% TFA in water/CH₃CN) to obtain the compound of Example 80 (6.5 mg, 30%).

MS: 388

### Example 81

### Synthesis of N-[(E)-3-(4′-hydroxy-5-trifluoromethyl-biphenyl-2-yl)-2-methyl-acryloyl]-guanidine

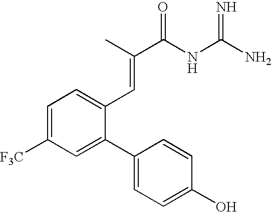 <Step 1>

NaH (60% assay, 237 mg, 5.92 mmol) was suspended in DMF (50 mL) and then cooled to 0° C. Triethyl 2-phosphonopropionate (1.29 mL, 5.92 mmol) in DMF (20 mL) was added dropwise in a slow manner to the resulting solution and stirred for 15 minutes. Then, 2-bromo-4-trifluoromethylbenzaldehyde (1.00 g, 3.95 mmol) in DMF (5 mL) was added thereto in a slow manner and stirred for 18 hours while gradually heating it from 0° C. to room temperature. EtOAc was added to the reaction solution, washed with water and saturated saline and then dried over anhydrous MgSO₄. The solvent was eliminated in vacuo to obtain a residue.

The resulting residue was dissolved in THF (30 mL) and MeOH (20 mL), 1 N NaOH (10 ml, 10 mmol) was added thereto and stirred at room temperature for 8 hours. The solvent was eliminated in vacuo, 2N HCl was added to acidify the solution and then the precipitated crystals were filtrated to obtain white crystals of the objective carboxylic acid (460 mg, 38%).

MS: 310

<Step 2>

The carboxylic acid obtained from Step 2 (460 mg, 1.49 mmol) was dissolved in DMF (20 mL), CDI (289 mg, 1.78 mmol) was added thereto and then stirred at room temperature for 30 minutes. N-Boc-guanidine (355 mg, 2.24 mmol) was added to the solution and then stirred for 16 hours. After the solvent was eliminated in vacuo, TFA (10 mL) was added to the residue at 0° C. and then stirred for 1.5 hours. After concentrating the solvent in vacuo, it was purified by reversed phase HPLC (0.1% TFA in water/CH₃CN) to obtain the objective acylguanidine (228 mg, 33%).

MS: 351

<Step 3>

The acylguanidine obtained from Step 3 (50 mg, 0.108 mmol) and 4-hydroxyphenyl boronic acid (16.4 mg, 0.119 mmol) were dissolved in a mixed solution of dioxane and water (v/v=3/1, 2.0 mL). Pd(PPh₃)₄ (6.24 mg, 5.40 μmol) and Na₂CO₃ (35.6 mg, 0.324 mmol) were added to the solution and then stirred at 90° C. for 2 hours. After cooling it to room temperature, the solvent was eliminated in vacuo and then purified by reversed phase HPLC (0.1% TFA in water/CH₃CN) to obtain the compound of Example 81 (19.2 mg, 37.3%).

1H-NMR (d-DMSO, 400 MHz) σ 1.96 (s, 3H), 6.63 (d, 2H, J=8.5 Hz), 7.21 (d, 3H, J=8.5 Hz), 7.37 (s, 1H), 7.64 (d, 2H, J=8.5 Hz), 7.68 (s, 1H), 7.76 (d, 1H, J=8.5 Hz), 8.36 (bs, 4H), 9.79 (s, 1H)

MS: 364

### Example 82

### Synthesis of N-[(E)-3-(3-fluoro-4′-hydroxy-biphenyl-2-yl)-2-methyl-acryloyl]-guanidine

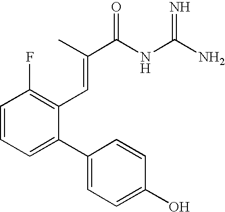 <Step 1>

An intermediate was obtained from 2-bromo-6-fluoro-benzaldehyde in the same manner as described for Intermediate 1.

MS: 300

<Step 2>

The intermediate obtained from Step 1 (93 mg, 0.225 mmol) and 4-hydroxyphenyl boronic acid (46.5 mg, 0.337 mmol) were dissolved in a mixed solution of dioxane and water (v/v=3/1, 4.0 mL). Pd(PPh₃)₄ (13.0 mg, 11.0 μmol) and Na₂CO₃ (95.4 mg, 0.90 mmol) were added to the solution and then stirred at 90° C. for 12 hours. After cooling it to room temperature, the solvent was eliminated in vacuo and then purified by reversed phase HPLC (0.1% TFA in water/CH₃CN) to obtain the compound of Example 82 (46.0 mg, 48%).

MS: 314

### Example 83

### Synthesis of N-{(E)-2-methyl-3-[2-(4-methyl-thiophene-3-yl)-phenyl]-acryloyl}-guanidine

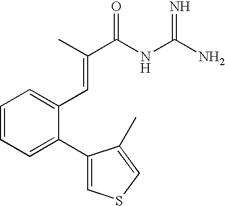 <Step 1>

Intermediate 1 (100 mg, 0.253 mmol) and 4-methylthiophene-3-boronic acid pinacol ester (85.0 mg, 0.379 mmol) were dissolved in a mixed solution of dioxane and water (v/v=3/1, 4.0 mL). Pd(PPh₃)₄ (14.6 mg, 13.0 μmol) and Na₂CO₃ (107.3 mg, 1.012 mmol) were added to the solution and then stirred at 80° C. for 12 hours. After cooling it to room temperature, the solvent was eliminated in vacuo and then purified by reversed phase HPLC (0.1% TFA in water/CH₃CN) to obtain the compound of Example 83.

MS: 300

### Example 84

### Synthesis of N-{(E)-2-methyl-3-[2-(1H-pyrrole-3-yl)-phenyl]-acryloyl}-guanidine

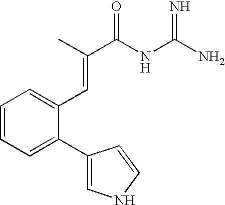 <Step 1>

Intermediate 1 (100 mg, 0.253 mmol) and 1-Boc-pyrrole-3-boronic acid pinacol ester (111 mg, 0.379 mmol) were dissolved in a mixed solution of dioxane and water (v/v=3/1, 4.0 mL). Pd(PPh₃)₄ (14.6 mg, 13.0 μmol) and Na₂CO₃ (107.3 mg, 1.012 mmol) were added to the solution and then stirred at 80° C. for 12 hours. After cooling it to room temperature, the solvent was eliminated in vacuo to obtain a residue. The resulting residue was stirred in TFA (5.0 mL) at room temperature for 30 minutes. After the solvent was eliminated in vacuo, it was purified by reversed phase HPLC (0.1% TFA in water/CH₃CN) to obtain the compound of Example 84.

MS: 269

### Example 85

### Synthesis of N-[(E)-3-(4-furan-3-yl-phenyl)-2-methyl-acryloyl]-guanidine

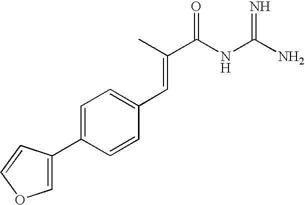 <Step 1>

Intermediate 5 (100 mg, 0.253 mmol) and furan-3-boronic acid (43.0 mg, 0.379 mmol) were dissolved in a mixed solution of dioxane and water (v/v=3/1, 4.0 mL). Pd(PPh₃)₄ (14.6 mg, 13.0 μmol) and Na₂CO₃ (107 mg, 1.01 mmol) were added to the solution and then stirred at 80° C. for 12 hours. After cooling it to room temperature, the solvent was eliminated in vacuo and then purified by reversed phase HPLC (0.1% TFA in water/CH—₃CN) to obtain the compound of Example 85 (54 mg, 55%).

MS: 270

### Example 86

### Synthesis of N-[(E)-3-(4′-hydroxy-2′-methyl-biphenyl-4-yl)-2-methyl-acryloyl]-guanidine

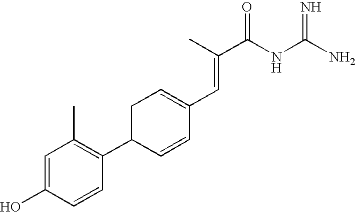 <Step 1>

Intermediate 5 (100 mg, 0.253 mmol) and 2-methyl-4-methoxyphenyl boronic acid (46.1 mg, 0.278 mmol) were dissolved in a mixed solution of dioxane and water (v/v=3/1, 4.0 mL). Pd(PPh₃)₄ (14.6 mg, 12.7 μmol) and Na₂CO₃ (80.5 mg, 0.759 mmol) were added to the solution and then stirred at 90° C. for 1.5 hours. After cooling it to room temperature, the solvent was eliminated in vacuo, EtOAc was added thereto, and the organic phase was washed with saturated NaHCO₃ solution, saturated saline and water and then dried over anhydrous MgSO₄. Then, the solvent was eliminated in vacuo to obtain a residue.

<Step 2>

CH₂Cl₂ (2.0 mL) was added to the residue obtained from Step 1 to dissolve, 1.0 mol/L BBr₃ dichloromethane solution (0.50 mL, 0.50 mmol) was added to the solution and then stirred at room temperature for 3 hours. After concentrating the solvent in vacuo, it was purified by reversed phase HPLC (0.1% TFA in water/CH₃CN) to obtain the compound of Example 86 (22.3 mg, 20.8%).

1H-NMR (d-DMSO, 400 MHz), σ 2.16 (d, 3H, J=1.2 Hz), 2.20 (s, 3H), 6.68 (dd, 1H, J=2.5, 8.3), 6.71 (dd, 1H, J=1.2, 2.2 Hz), 7.05 (d, 1H, J=8.3 Hz), 7.41 (d, 2H, J=8.3 Hz), 7.52 (s, 1H), 7.56 (d, 2H, J=8.3 Hz), 8.39 (bs, 4H), 9.45 (s, 1H), 11.1 (s, 1H)

MS: 310

### Example 87

### Synthesis of N-[(E)-3-(3′-hydroxy-biphenyl-4-yl)-2-methyl-acryloyl]-guanidine

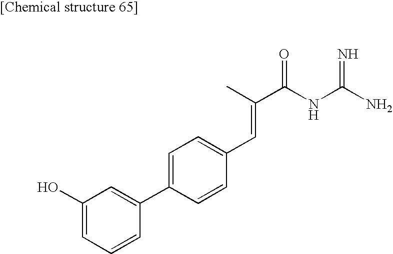 <Step 1>

Intermediate 5 and 3-hydroxyphenyl boronic acid were reacted to obtain the compound of Example 87 in the same manner as described for Example 1.

MS: 296

### Example 88

### Synthesis of N-[3-(2′-hydroxy-biphenyl-4-yl)-2-methyl-acryloyl]-guanidine

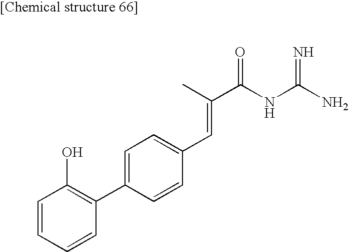 <Step 1>

Intermediate 5 and 2-hydroxyphenyl boronic acid were reacted to obtain the compound of Example 88 in the same manner as described for Example 1.

MS: 296

### Example 89

### Synthesis of N-[(E)-3-(4′-fluoro-3′-hydroxy-biphenyl-4-yl)-2-methyl-acryloyl]-guanidine

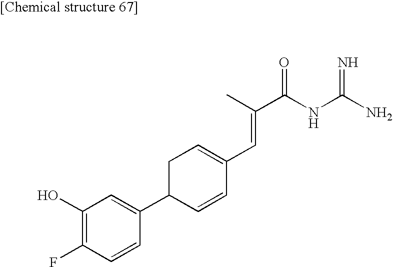 <Step 1>

Intermediate 5 (50 mg, 0.126 mmol) and 4-fluoro-3-hydroxyphenyl boronic acid (21.7 mg, 0.139 mmol) were dissolved in a mixed solution of dioxane and water (v/v=3/1, 2.0 mL). Pd(PPh₃)₄ (7.23 mg, 6.3 μmol) and Na₂CO₃ (40.1 mg, 0.378 mmol) were added to the solution and then stirred at 90° C. for 2 hours. After cooling it to room temperature, the solvent was eliminated in vacuo and then purified by reversed phase HPLC (0.1% TFA in water/CH₃CN) to obtain the compound of Example 89 (10.2 mg, 18.9%).

MS: 314

### Example 90

### Synthesis of N-[(E)-3-(4′-hydroxy-2′-methyl-biphenyl-4-yl)-2-methyl-acryloyl]-guanidine

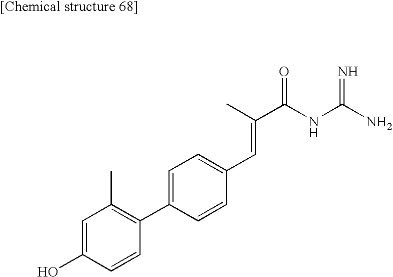 <Step 1>

Intermediate 5 (100 mg, 0.253 mmol) and 2-methyl-4-methoxyphenyl boronic acid (46.1 mg, 0.278 mmol) were dissolved in a mixed solution of dioxane and water (v/v=3/1, 4.0 mL). Pd(PPh₃)₄ (14.6 mg, 12.7 μmol) and Na₂CO₃ (80.5 mg, 0.759 mmol) were added to the solution and then stirred at 90° C. for 1.5 hours. After cooling it to room temperature, the solvent was eliminated in vacuo, EtOAc was added thereto, and the organic phase was washed with saturated NaHCO₃ solution and saturated saline and then dried over anhydrous MgSO₄. Then, the solvent was eliminated in vacuo to obtain a residue. CH₂Cl₂ (2.0 mL) was added to the resulting residue, 1.0 mol/L BBr₃ dichloromethane solution (0.50 mL, 0.50 mmol) was added to the solution and then stirred at room temperature for 3 hours. After concentrating the solvent in vacuo, it was purified by reversed phase HPLC (0.1% TFA in water/CH₃CN) to obtain the compound of Example 90 (22.3 mg, 20.8%).

MS: 310

### Example 91

### Synthesis of N-[(E)-3-(2′-fluoro-5′-hydroxy-biphenyl-4-yl)-2-methyl-acryloyl]-guanidine

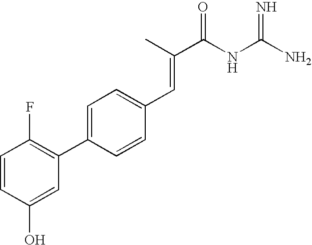 <Step 1>

Intermediate 5 (100 mg, 0.253 mmol) and 2-fluoro-5-methoxuphenyl boronic acid (64.4 mg, 0.379 mmol) were dissolved in a mixed solution of dioxane and water (v/v=3/1, 4.0 mL). Pd(PPh₃)₄ (14.6 mg, 13.0 μmol) and Na₂CO₃ (107 mg, 1.01 mmol) were added to the solution and then stirred at 80° C. for 12 hours. After cooling it to room temperature, the solvent was eliminated in vacuo and then it was purified by reversed phase HPLC (0.1% TFA in water/CH₃CN) to obtain the objective intermediate (112 mg, 100%).

MS: 328

<Step 2>

1.0 mol/L BBr₃ dichloromethane solution (2.53 mL, 2.53 mmol) was added at 0° C. to the intermediate obtained from Step 1 (112 mg, 0.253 mmol) and then stirred at room temperature for 3 hours. After cooling it to 0° C., it was diluted with dichloromethane and then water was added to terminate the reaction. After the solvent was eliminated in vacuo, it was purified by reversed phase HPLC (0.1% TFA in water/CH₃CN) to obtain the compound of Example 91 (77.0 mg, 70%).

MS: 314

### Example 92

### Synthesis of N-[(E)-3-(3′,4′-dihydroxy-biphenyl-4-yl)-2-methyl-acryloyl]-guanidine

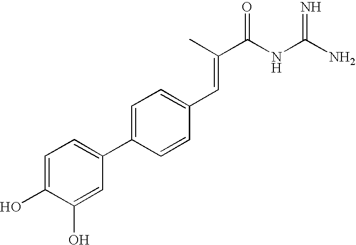 <Step 1>

Intermediate 5 (100 mg, 0.253 mmol) and 3-methoxy-4-hydroxyphenyl boronic acid pinacol ester (95.3 mg, 0.381 mmol) were dissolved in a mixed solution of dioxane and water (v/v=3/1, 4.0 mL). Pd(PPh₃)₄ (14.6 mg, 13.0 μmol) and Na₂CO₃ (107 mg, 1.01 mmol) were added to the solution and then stirred at 80° C. for 6 hours. After cooling it to room temperature, the solvent was eliminated in vacuo and then it was purified by reversed phase HPLC (0.1% TFA in water/CH₃CN) to obtain the objective intermediate (60.0 mg, 54%).

MS: 326

<Step 2>

1.0 mol/L BBr₃ dichloromethane solution (0.46 mL, 0.46 mmol) was added at 0° C. to the intermediate obtained from Step 1 (20 mg, 0.046 mmol) and then stirred at room temperature for 12 hours. After cooling it to 0° C., it was diluted with dichloromethane and then water was added to terminate the reaction. After the solvent was eliminated in vacuo, it was purified by reversed phase HPLC (0.1% TFA in water/CH₃CN) to obtain the compound of Example 92 (10.0 mg, 51%).

MS: 312

### Example 93

### Synthesis of N-[(E)-3-(4′-hydroxy-biphenyl-4-yl)-2-methyl-acryloyl]-guanidine

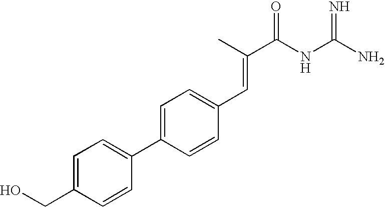 <Step 1>

Intermediate 5 (50 mg, 0.127 mmol) and 4-hydroxymethylphenyl boronic acid (29.0 mg, 0.190 mmol) were dissolved in a mixed solution of dioxane and water (v/v=3/1, 2.5 mL). Pd(PPh₃)₄ (7.50 mg, 7.0 μmol) and Na₂CO₃ (54.0 mg, 0.51 mmol) were added to the solution and then stirred at 80° C. for 12 hours. After cooling it to room temperature, the solvent was eliminated in vacuo and then it was purified by reversed phase HPLC (0.1% TFA in water/CH₃CN) to obtain the compound of Example 93 (43 mg, 80%).

MS: 310

### Example 94

### Synthesis of N-[(E)-3-(4′-hydroxy-3-morpholine-4-yl-biphenyl-4-yl)-2-methyl-acryloyl]-guanidine

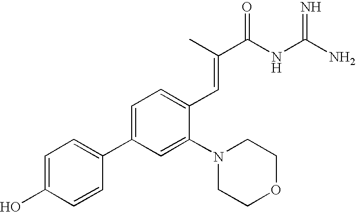

NaH (60% assay, 222 mg, 5.55 mmol) was suspended in THF (20 mL), and triethyl-2-phosphonopropionate (1.19 mL, 5.55 mmol) was added dropwise in a slow manner to the solution and then stirred for 30 minutes. Then, 4-bromo-2-(N-morpholino)-benzaldehyde (500 mg, 1.85 mmol) was added in a slow manner and then stirred for 12 hours. EtOAc was then added to the reaction solution, washed with water and saturated saline and then dried over anhydrous MgSO₄. Then, the solvent was eliminated in vacuo to obtain an ester which was a crud, product. The resulting compound was dissolved in THF (5 mL) and MeOH (3 mL), 2 N NaOH (3 mL, 16.0 mmol) was added thereto and then stirred at 50° C. for 30 minutes. Then, the solvent was eliminated in vacuo, 2 N HCl was added to acidify the solution and then extracted with dichloromethane. After the solvent was eliminated, it was dried over Na₂SO₄ to obtain the objective carboxylic acid.

MS: 326

<Step 2>

The carboxylic acid obtained from Step 1 was dissolved in DMF (15.0 mL), CDI (360 mg, 2.22 mmol) was added thereto and then stirred at room temperature for 30 minutes. N-Boc-guanidine (355 mg, 2.22 mmol) was added to the solution and then stirred for 12 hours. Then, EtOAc was added thereto, washed with water and saturated saline and then dried over Na₂SO₄. TFA (15 mL) was added to the residue and then stirred for 1 hour. After concentrating the solvent in vacuo, it was purified by reversed phase HPLC (0.1% TFA in water/CH₃CN) to obtain an intermediate (300 mg, 34%).

MS: 367

<Step 3>

The intermediate obtained from Step 2 (50.0 mg, 0.104 mmol) and 4-hydroxyphenyl boronic acid (21.5 mg, 0.156 mmol) were dissolved in a mixed solution of dioxane and water (v/v=4/1, 2.5 mL). Pd(PPh₃)₄ (6.0 mg, 5.0 μmol) and Na₂CO₃ (44.0 mg, 0.416 mmol) were added to the solution and then stirred at 80° C. for 12 hours. After cooling it to room temperature, the solvent was eliminated in vacuo and then it was purified by reversed phase HPLC (0.1% TFA in water/CH₃CN) to obtain the compound of Example 94 (41.0 mg, 80%).

MS: 381

### Example 95

### Synthesis of N-[(E)-3-(4′-hydroxy-3-methyl-biphenyl-4-yl)-2-methyl-acryloyl]-guanidine

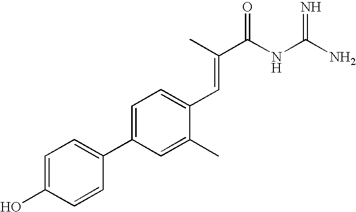 <Step 1>

Intermediate 6 (50.0 mg, 0.122 mmol) and 4-hydroxyphenyl boronic acid (25.2 mg, 0.183 mmol) were dissolved in a mixed solution of dioxane and water (v/v=3/1, 4.0 mL). Pd(PPh₃)_{4 (}7.0 mg, 6.0 μmol) and Na₂CO₃ (51.7 mg, 0.488 mmol) were added to the solution and then stirred at 80° C. for 12 hours. After cooling it to room temperature, the solvent was eliminated in vacuo and then it was purified by reversed phase HPLC (0.1% TFA in water/CH₃CN) to obtain the compound of Example 95 (31 mg, 60%).

MS: 324

### Example 96

### Synthesis of N-[(E)-3-(4′-hydroxy-3,2′-dimethyl-biphenyl-4-yl)-2-methyl-acryloyl]-guanidine

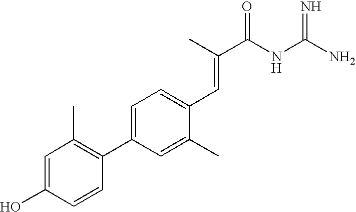 <Step 1>

Intermediate 6 (100 mg, 0.244 mmol) and 2-methyl-4-methoxyphenyl boronic acid (60.8 mg, 0.366 mmol) were dissolved in a mixed solution of dioxane and water (v/v=3/1, 4.0 mL). Pd(PPh₃)₄ (14.0 mg, 12.0 μmol) and Na₂CO₃ (103.4 mg, 0.976 mmol) were added to the solution and then stirred at 80° C. for 12 hours. After cooling it to room temperature, the solvent was eliminated in vacuo and then it was purified by reversed phase HPLC (0.1% TFA in water/CH₃CN) to obtain the objective intermediate (55 mg, 50%).

MS: 338

<Step 2>

1.0 mol/L BBr₃ dichloromethane solution (1.10 mL, 1.10 mmol) was added to the intermediate obtained from Step 1 (50 mg, 0.11 mmol) at 0° C. and then stirred at room temperature for 3 hours. After cooling it to 0° C., it was diluted with dichloromethane and then water was added to terminate the reaction. After the solvent was eliminated in vacuo, it was purified by reversed phase HPLC (0.1% TFA in water/CH₃CN) to obtain the compound of Example 96 (24.0 mg, 50%).

MS: 324

### Example 97

### Synthesis of (E)-2-methyl-2-phenyl-propionic acid 2′-(3-guanidino-2-methyl-3-oxo-propenyl)-biphenyl-4-yl ester

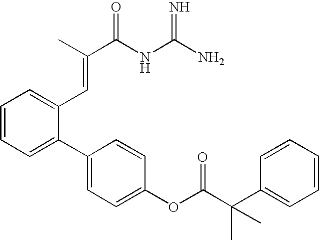 <Step 1>

NaH (60% assay, 944 mg, 23.6 mmol) was suspended in DMF (50 mL) and then cooled to 0° C. 2-(diethoxy-phosphoryl)-propionic acid tert-butyl ester (5.9 mL, 23.6 mmol) in DMF (10 mL) was added dropwise in a slow manner to the resulting solution and stirred for 15 minutes. Then, 2-bromobenzaldehyde (3.5 g, 18.9 mmol) in DMF (3 mL) was added thereto in a slow manner thereto and stirred for 18 hours while gradually heating it from 0° C. to room temperature. EtOAc was added to the reaction solution, washed with water and saturated saline and then dried over anhydrous MgSO₄. After the solvent was eliminated in vacuo, it was purified by silica gel column chromatography (SiO₂, Hexane/EtOAc) to obtain the objective ester compound (4.84 g, 86%).

MS: 298

<Step 2>

The ester obtained from Step 1 (3.59 g, 12 mmol) and 4-hydroxyphenyl boronic acid (2.0 g, 14.5 mmol) were dissolved in a mixed solution of dioxane and water (v/v=3/1, 2.4 mL). Pd(PPh₃)₄ (168 mg, 145 μmol) and Na₂CO₃ (2.5 g, 24 mmol) were added to the solution and then stirred at 90° C. for 15.5 hours. After cooling it to room temperature, the solvent was eliminated in vacuo and then it was purified by reversed phase HPLC (0.1% TFA in water/CH₃CN) to obtain a coupling product (1.9 g, 44%).

MS: 241

<Step 3>

Dichloromethane (5 mL), HATU (170 mg, 0.46 mmol), triethylamine (0.09 mL, 0.778 mmol) and 2-methyl-2-phenylpropionic acid (67 mg, 0.41 mmol) were added to the coupling product obtained from Step 2 (115 mg, 0.322 mmol) and then stirred overnight at room temperature. After the solvent was eliminated in vacuo, the resulting residue was dissolved in TFA (3.0 mL) at 0° C. and then stirred at room temperature for 2 hours. After the solvent was eliminated in vacuo, ethylacetate was added thereto; and the organic phase was washed with saturated NaHCO₃ solution and saturated saline and then dried over anhydrous MgSO₄. A crude product was obtained by eliminating the solvent in vacuo. The resulting crude product was then dissolved in DMF (3.0 mL), CDI (38 mg, 0.23 mmol) was added thereto and then stirred at room temperature for 30 minutes. N-Boc-guanidine (48 mg, 0.25 mmol) was added to the solution and then stirred overnight. After the solvent was eliminated in vacuo, TFA (3.0 mL) was added to the residue and then stirred for 4.5 hours. After concentrating the solvent in vacuo, it was purified by reversed phase HPLC (0.1% TFA in water/CH₃CN) to obtain the compound of Example 97 (5 mg, 3%).

MS: 442

Structural formulae of the compounds demonstrated in Examples will be shown in Tables 1, 2, 3, 4, 5 and 6.

TABLE 1

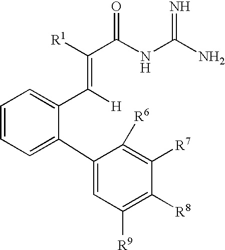

| Example | R¹ | R⁶ | R⁷ | R⁸ | R⁹ | MS |
|---|---|---|---|---|---|---|
| 1 | Me | H | H | Cl | H | 314 |
| 2 | Me | H | H | OH | H | 296 |
| 3 | Me | H | H | OMe | H | 310 |
| 4 | Me | H | H | OEt | H | 324 |
| 5 | Me | H | H | COMe | H | 322 |
| 6 | Me | H | H | CH₂OH | H | 310 |
| 7 | Me | H | H | CO₂Me | H | 338 |
| 8 | Me | H | H | NHSO₂Me | H | 373 |
| 9 | Me | H | H | CONH₂ | H | 323 |
| 10 | Me | H | H | CO₂H | H | 324 |
| 11 | Me | H | H | B(OH)₂ | H | 324 |
| 12 | Me | H | H | NO₂ | H | 325 |
| 13 | Me | H | OH | H | H | 296 |
| 14 | Me | H | 1-hydroxy- | H | H | 324 |
| | | | ethyl | | | |
| 15 | Me | H | COMe | H | H | 322 |
| 16 | Me | H | CO₂Me | H | H | 338 |
| 17 | Me | H | NHSO₂Me | H | H | 373 |
| 18 | Me | H | CONH₂ | H | H | 323 |
| 19 | Me | H | CO₂H | H | H | 324 |
| 20 | Me | H | CN | H | H | 305 |
| 21 | Me | OH | H | H | H | 296 |
| 22 | Me | H | Me | OH | Me | 324 |
| 23 | Me | H | OMe | OH | H | 326 |
| 24 | Me | H | F | OH | H | 314 |
| 25 | H | H | F | OH | F | 318 |
| 26 | Me | H | OH | OH | H | 312 |
| 27 | Me | H | OH | H | OH | 312 |
| 28 | Me | H | OH | OH | OH | 328 |
| 29 | Me | Me | H | OH | H | 296 |
| 30 | Me | H | —OCH₂O— | | H | 324 |
| 31 | Me | H | —OCH₂CH₂O— | | H | 338 |
| 32 | H | H | H | OH | H | 282 |
| 33 | H | H | OH | H | H | 282 |
| 34 | Et | H | OH | H | H | 310 |
| 51 | Me | NHSO₂Me | H | H | H | 373 |
| 52 | Me | OMe | OMe | H | H | 340 |
| 53 | Me | OH | H | OH | H | 312 |
| 54 | Me | F | H | OH | F | 332 |
| 55 | Me | H | CONHOH | H | H | 339 |
| 56 | Me | H | H | CNHNH₂ | H | 322 |
| 57 | Me | Me | H | CNHNH₂ | H | 336 |
| 58 | Me | H | F | CNHNH₂ | H | 340 |
| 59 | Me | F | H | OH | H | 314 |
| 60 | Me | Cl | H | OH | H | 331 |
| 61 | Me | H | Me | OH | H | 297 |

TABLE 2

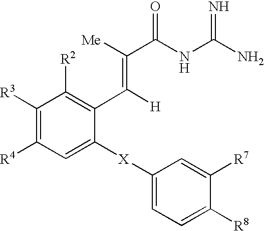

| Example | R² | R³ | R⁴ | R⁷ | R⁸ | X | MS |
|---|---|---|---|---|---|---|---|
| 35 | H | OH | H | OH | H | — | 312 |
| 36 | H | OH | OH | OH | H | — | 328 |
| 37 | H | H | Me | OH | H | — | 310 |
| 38 | H | Me | H | OH | H | — | 310 |
| 39 | H | OMe | OMe | OH | H | — | 356 |
| 40 | 3-HO—Ph | H | H | OH | H | — | 388 |
| 41 | H | 3-HO—Ph | H | OH | H | — | 388 |
| 42 | H | H | F | OH | H | — | 314 |
| 43 | H | H | F | H | OH | — | 314 |
| 44 | H | OH | H | H | OH | — | 312 |
| 45 | H | OMe | OMe | H | OH | — | 356 |
| 46 | H | OH | OH | H | OH | — | 328 |
| 47 | H | H | Me | H | OH | — | 310 |
| 48 | H | 4-HO—Ph | H | H | OH | — | 388 |
| 49 | H | H | H | OMe | H | O | 326 |
| 50 | H | H | H | OH | H | O | 312 |

TABLE 3

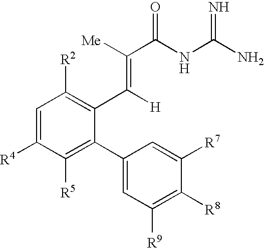

| Example | R² | R⁴ | R⁵ | R⁷ | R⁸ | R⁹ | MS |
|---|---|---|---|---|---|---|---|
| 62 | H | Me | H | F | OH | H | 328 |
| 63 | H | Me | H | OH | F | H | 328 |
| 64 | H | 4-SO₂NH₂—PhO— | H | OH | H | H | 477 |
| 65 | H | 4-SO₂NH₂—PhO— | H | H | OH | H | 477 |
| 66 | H | Me | H | OMe | OH | H | 340 |
| 67 | H | H | Me | H | OH | H | 310 |
| 68 | H | H | Me | OH | H | H | 310 |
| 69 | H | Me | H | OH | OH | H | 326 |
| 70 | Br | H | H | H | OH | H | 376 |
| 71 | H | 4-OH—PhO— | H | H | OH | H | 404 |
| 72 | H | 4-OH—Ph— | H | H | OH | H | 388 |
| 73 | H | OMe | H | H | OH | H | 326 |
| 74 | H | Me | H | OH | OMe | H | 340 |
| 75 | H | 3,5-diMe-4-OH—Ph— | H | Me | OH | Me | 444 |
| 76 | H | 3-OH-4-F—Ph— | H | OH | F | H | 424 |
| 77 | H | 3-OH—Ph— | H | OH | H | H | 388 |
| 78 | H | 3-OMe-4-OH—Ph— | H | OMe | OH | H | 448 |
| 79 | H | Me | H | F | CHNHNH₂ | H | 354 |
| 80 | 4-OH—Ph— | H | H | H | OH | H | 388 |
| 81 | H | CF₃ | H | H | OH | H | 364 |
| 82 | F | H | H | H | OH | H | 314 |

TABLE 4

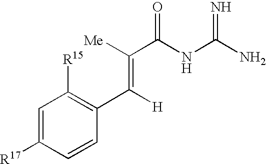

| Example | R¹⁵ | R¹⁷ | MS |
|---|---|---|---|
| 83 | 4-methyl-thiophen-3-yl | H | 300 |
| 84 | 1H-pyrrol-3-yl | H | 269 |
| 85 | H | 4-furan-3-yl | 270 |

TABLE 5

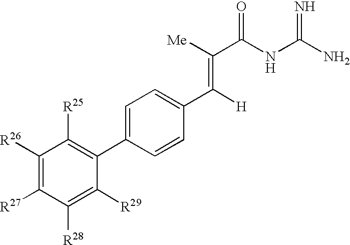

| Example | R²⁵ | R²⁶ | R²⁷ | R²⁸ | MS |
|---|---|---|---|---|---|
| 86 | H | MeSO₂NH— | H | H | 373 |
| 87 | H | OH | H | H | 296 |
| 88 | OH | H | H | H | 296 |
| 89 | H | OH | F | H | 314 |
| 90 | Me | H | OH | H | 310 |
| 91 | F | H | H | OH | 314 |
| 92 | H | OH | OH | H | 312 |
| 93 | H | H | OH₂OH | H | 310 |

TABLE 6

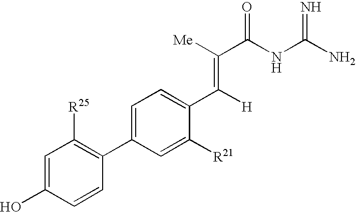

| Example | R²¹ | R²⁵ | MS |
|---|---|---|---|
| 94 | morphorine-4-yl | H | 381 |
| 95 | Me | H | 310 |
| 96 | Me | Me | 324 |

Compounds according to the present invention were examined for NHE inhibitory activities by using the following methods.

Pharmacological Test Example 1: Measurements of NHE1 Inhibitory Activities

HLF cells (Human hepatoma cell line) were used as cells for the test. After 1×10⁴ cells/well were seeded to a 96-well plate and cultured for 3 days, they were cultured overnight under a serum-free medium. Then, the cells were incubated in a stain solution of Tetramethylammonium (TMA) Buffer (130 mM TMA-C1, 5 mM KCl, 2 mM CaCl₂, 1 mM MgSO₂, 25 mM glucose, 20 mM HEPES; pH 7.4) containing 40 mM NH₄Cl and 1 μg/mL pH-sensitive fluorescent indicator BCECF-AM at 37° C. for 40 minutes and then BCECF was introduced into the cells. After the cells were washed once with TMA Buffer and incubated in TMA+40 mM NH₄Cl solution at 37° C. for 15 minutes, the solution was removed and 20 μL/well of TMA Buffer and 10 μL/well of each test compound solution prepared with TMA Buffer were added to each well. The measurements were carried out by adding 200 μL/well of Na Buffer (130 mM, NaCl, 5 mM KCl, 2 mM CaCl₂, 1 mM MgSO₂, 1 mM NaH₂PO₄, 25 mM glucose, 20 mM HEPES; pH 7.4) or TMA Buffer (for base measurement), immediately placing it on FlexStation (Molecular Device) and then, after 10 minutes, measuring, it the two wavelengths of 505 nm (excitation wavelength)/530 nm (emission wavelength) (measurement value) and 440 nm (excitation wavelength)/530 nm (emission wavelength) (isosbestic point value). A NHE activity was calculated by dividing a measurement value by an isosbestic point value and a NHE1 inhibitory activity was calculated by the following equation.
NHE1 inhibitory activity (%)=100×(1−(Measurement value [addition of each compound]−Base [addition of TMA Buffer])/(Control [addition of Na Buffer]−Base [addition of TMA Buffer]))

Evaluation results of NHE1 inhibitory activities for representative compounds according to the present invention are shown in Table 7.

TABLE 7

| Example | hNHE1 IC₅₀(uM) |
|---|---|
| 11 | 1.2 |
| 15 | 1.2 |
| 22 | 3.1 |
| 23 | 2.6 |
| 24 | 1.2 |
| 26 | 0.5 |
| 37 | 1.6 |
| 47 | 1.6 |
| 53 | 2.9 |
| 54 | 7.6 |
| 55 | 2.9 |
| 56 | 3.4 |
| 57 | 1.1 |
| 58 | 0.2 |
| 59 | 3.2 |
| 60 | 0.91 |
| 61 | 4.3 |
| 62 | 1.3 |
| 63 | 9.9 |
| 64 | 1.3 |
| 65 | 1.5 |
| 67 | 2.1 |
| 68 | 1.7 |
| 69 | 0.58 |
| 71 | 1.3 |
| 72 | 1.1 |
| 74 | 1.4 |
| 75 | 1.8 |
| 76 | 1.1 |
| 77 | 0.94 |
| 78 | 3.3 |
| 79 | 5.2 |
| 80 | 7.7 |
| 81 | 5.2 |
| 82 | 6.6 |
| 83 | 2.2 |
| 84 | 2.1 |
| 94 | 2.1 |

Pharmacological Test Example 2: Measurements of NHE3 Inhibitory Activities.

OK26 cells (OK (opossum kidney) cells in which human NHE3 genes are overexpressed) were used as cells for the test. The measurements were carried out in the same manner as described in Pharmacological Test Example 1 except that the measurement time was 5 minutes (at 37° C.) to calculate NHE3 inhibitory activities.

Evaluation results of NHE3 inhibitory activities for representative compounds according to the present invention are shown in Table 8.

### Example 8

| Example | hNHE3 IC₅₀(uM) |
|---|---|
| 2 | 0.11 |
| 7 | >10 |
| 10 | 1.6 |
| 11 | 0.23 |
| 13 | 0.21 |
| 15 | 0.61 |
| 22 | 0.23 |
| 24 | 0.47 |
| 26 | 0.23 |
| 36 | 0.32 |
| 37 | 0.18 |
| 47 | 0.19 |
| 86 | 0.23 |
| 87 | 0.27 |
| 88 | 0.48 |
| 89 | 0.86 |

Pharmacological Test Example 3: Improved Version of Measurements of NHE3 Inhibitory Activities

OK26 cells express endogeneous NHE1 (opNHE1). In order to evaluate NHE3 inhibitory activities more precisely, OK26 ND cell lines in which the expression level of opNHE1 is reduced by 90% were established. Measurements of NEE inhibitory activities were carried out by using the established cell lines in the same manner as described in Pharmacological Test Example 1 except that the measurement time was 8 minutes (at 26° C.) to calculate more precise NHE3 inhibitory activities.

Evaluation results of NHE3 inhibitory activities for representative compounds according to the present invention are shown in Table 9.

### Example 9

| Example | hNHE3 IC₅₀(uM) |
|---|---|
| 2 | 0.083 |
| 6 | 0.4 |
| 11 | 0.1 |
| 13 | 0.11 |
| 15 | 0.36 |
| 22 | 0.23 |
| 23 | 0.61 |
| 24 | 0.21 |
| 26 | 0.23 |
| 36 | 0.23 |
| 37 | 0.038 |
| 47 | 0.06 |
| 53 | 0.74 |
| 54 | 0.24 |
| 55 | 0.44 |
| 56 | 0.4 |
| 57 | 0.1 |
| 58 | 0.009 |
| 59 | 0.28 |
| 60 | 0.31 |
| 61 | 0.4 |
| 62 | 0.092 |
| 63 | 0.33 |
| 64 | 0.15 |
| 65 | 0.21 |
| 66 | 0.13 |
| 67 | 0.12 |
| 68 | 0.12 |
| 69 | 0.1 |
| 70 | 0.96 |
| 71 | 0.13 |
| 72 | 0.087 |
| 73 | 0.12 |
| 74 | 0.2 |
| 75 | 0.1 |
| 76 | 0.4 |
| 77 | 0.061 |
| 78 | 0.15 |
| 79 | 0.7 |
| 80 | 0.2 |
| 81 | 0.63 |
| 82 | 0.35 |
| 83 | 0.54 |
| 84 | 0.18 |
| 85 | 0.47 |
| 90 | 0.3 |
| 91 | 0.19 |
| 92 | 0.15 |
| 93 | 0.25 |
| 94 | 0.32 |
| 95 | 0.24 |
| 96 | 0.2 |

Evaluations for membrane permeability of the present compound were conducted by using MDCK (MADIN-DARBY Canine Kidney) cells.

Pharmacological Test Example 4: MDCK Membrane Permeability

1×10⁶ MDCK (MADIN-DARBY Canine Kidney) cells were seeded to each well and cultivated on a trans-well for 4 days (Mixed medium; DMEM: F12=1:1). The trans-well consists of a upper chamber into which cells are seeded and a lower chamber which is separated by a porous membrane and each test compound added into the upper chamber penetrates through the porous membrane to be detected in the lower chamber. The trans-well system has been used as a model for cell membrane permeability.

Buffer solution (pH 6.5) (138 mM NaCl, 2.7 mM KCl, 25 mM D-Glucose, 20 mM MES, 1.25 mM CaCl₂, 0.5 mM MgCl₂; pH was adjusted with KOH) was added into the upper chamber (Apical side) while Buffer solution (pH 7.4) (138 mM NaCl, 2.7 mM KCl, 25 mM D-Glucose, 20 mM HEPES, 1.25 mM CaCl₂, 0.5 mM MgCl₂; pH was adjusted with KOH) was added into the lower chamber (basal side). After it was pre-incubated at 37° C. for 20 minutes, 50 μM of each test compound was added thereto and then reacted at 37° C. for 1 hour. The solutions in the upper and lower chambers were collected and the concentrations of each test compound were determined by LC/MS to calculate membrane permeability values (Pₘ values) by the following equation.
Pₘ[cm/sec]=(Concentration of each test compound in the basal side×1.5 mL)/(3600 sec×1.12 cm²×Initial concentration of the added compound)

Membrane permeability values (Pₘ values) of the compounds of Examples 7 and 15 are shown in Table 10.

TABLE 10

| Example | Pₘ value (cm/sec) |
|---|---|
| 7 | 2 × 10⁻⁶ |
| 15 | 1 × 10⁻⁶ |

Continuous administration test by using renal dysfunction model rats was carried out to determine improving effects on renal dysfunction of the present compound.

Pharmacological Test Example 5: Continuous Administration Test in Renal Dysfunction Model Rats

After unilateral nephrectomy was performed on 7-week-old Wistar rats and the rats were habituated for 1 week, they were divided into 4 groups based on their body weights, Normal group, Vehicle group, 20 mg/kg of the compound of Example 7-administered group and 50 mg/kg of the compound of Example 7-administered group were assigned to 5 rats, respectively. After the rats were habituated in metabolic cages for 4 days, the test compound the compound of Example 7) dissolved in 0.5% methylcellulose solution was administered via gavage simultaneously with intraperitoneal administration of oleic acid-containing bovine serum albumin (OA-BSA) at a dose of 2 g/animal once daily for 4 days. Vehicles (0.5% methylcellulose solution) were administered to Normal group and Vehicle group instead of the test compound (the compound of Example 7). In addition, no oleic acid-containing bovine serum albumin was administered to Normal group and only unilateral nephrectomy was performed on the Normal group rats. Urine samples were collected from each rat on the last administration day to the next day of the last administration, and blood sampling and autopsy were conducted on the same day. A result for beta 2-microglobulin, which is a marker for tubular damage, after 4 days administration is shown in FIG. 1. Significant improvements in tubular damages were observed in 20 mg/kg and 50 mg/kg of the test compound (the compound of Example 7)-administered groups. Renal pathological images are shown in FIG. 2 and a graph of tubular damage score is shown in FIG. 3. Renal dysfunction images associated with OA-BSA administrations such as dilated renal tubule and appearances of urinary cast were observed in Vehicle group and the tubular damage score was significantly increased compared to Normal group. On the other hand, improvements in dilation of proximal renal tubules and significant reductions in appearances of urinary cast were observed in the test compound (the compound of Example 7)-administered groups. Reductions in tubular damage scores were also observed in the test compound administered groups. These results show that renal dysfunction was improved by the administration of the NHE3 inhibitors.

What is claimed is:

1. A compound of the following formula (I) or a pharmaceutically acceptable salt thereof: 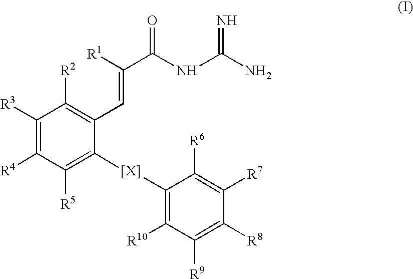
wherein
R¹ is a hydrogen atom, a halogen atom, a substituted or unsubstituted C₁₋₆-alkyl group;
R², R³, R⁴ and R⁵ are each independently selected from the group consisting of a hydrogen atom, a halogen atom, a hydroxy group, a substituted or unsubstituted C₁₋₆-alkyl group, a substituted or unsubstituted C₁₋₆-alkenyl group, a substituted or unsubstituted C₁₋₆-alkynyl group, a substituted or unsubstituted C₁₋₆-alkoxy group, a substituted or unsubstituted C₁₋₆-alkylthio group, a substituted or unsubstituted phenyloxy group and a substituted or unsubstituted phenyl group;
X is a single bond, —O— or —S—;
R⁶, R⁷, R⁸, R⁹ and R¹⁰ are each independently selected from the group consisting of a hydrogen atom, a halogen atom, a nitro group, a nitrile group, a carboxyl group, a hydroxy group, —B(OH)₂, a substituted or unsubstituted amidino group, a substituted or unsubstituted C₁₋₆-alkyl group, a substituted or unsubstituted C₁₋₆-alkenyl group, a substituted or unsubstituted C₁₋₆-alkynyl group, a substituted or unsubstituted C₁₋₆-alkoxy group, a substituted or unsubstituted C₁₋₆-alkylthio group, a substituted or unsubstituted aminocarbonyl, a substituted or unsubstituted C₁₋₆-alkyl-carbonyl group, a substituted or unsubstituted C₁₋₆-alkoxy-carbonyl group, a substituted or unsubstituted C₁₋₆-alkyl-S(═O)₂—NH group and —OP, or
two adjacent groups from R⁶, R⁷, R⁸ and R⁹ together may form a 5-membered or 6-membered heterocyclic ring which has one or two oxygen atom(s) as a hetero atom(s) constituting the ring; and
P is selected from the group consisting of a substituted or unsubstituted C₁₋₆-acyl group, a substituted or unsubstituted C₁₋₆-alkoxycarbonyl group and a substituted or unsubstituted C₁₋₆-alkylaminocarbonyl group.

2. The compound according to claim 1 or a pharmaceutically acceptable salt thereof wherein R², R³, R⁴ and R⁵ are each independently selected from the group consisting of a hydrogen atom, a halogen atom, a hydroxy group, a substituted or unsubstituted C₁₋₆-alkyl group, a substituted or unsubstituted C₁₋₆-alkenyl group, a substituted or unsubstituted C₁₋₆-alkynyl group, a substituted or unsubstituted C₁₋₆-alkoxy group, a substituted or unsubstituted C₁₋₆-alkylthio group and a substituted or unsubstituted phenyl group; and
R⁶, R⁷, R⁸, R⁹ and R¹⁰ are each independently selected from the group consisting of a hydrogen atom, a halogen atom, a nitro group, a nitrile group, a carboxyl group, a hydroxy group, —B(OH)₂, a substituted or unsubstituted C₁₋₆-alkyl group, a substituted or unsubstituted C₁₋₆-alkenyl group, a substituted or unsubstituted C₁₋₆-alkynyl group, a substituted or unsubstituted C₁₋₆-alkoxy group, a substituted or unsubstituted C₁₋₆-alkylthio group, an aminocarbonyl, a substituted or unsubstituted C₁₋₆-alkylcarbonyl group, a substituted or unsubstituted C₁₋₆-alkoxycarbonyl group and a substituted or unsubstituted C₁₋₆-alkyl-S(═O)₂—NH group, or
two adjacent groups from R⁶, R⁷, R⁸ and R⁹ together may form a 5-membered or 6-membered heterocyclic ring which has one or two oxygen atom(s) as a hetero atom(s) constituting the ring.

3. The compound according to claim 1 or a pharmaceutically acceptable salt thereof wherein X is a single bond or —O—.

4. The compound according to claim 1 or a pharmaceutically acceptable salt thereof wherein X is a single bond.

5. The compound according to claim 1 or a pharmaceutically acceptable salt thereof wherein R⁵ is a hydrogen atom or a methyl group; and
R⁶ and R¹⁰ are each independently selected from the group consisting of a hydrogen atom, a halogen atom, a hydroxy group and a substituted or unsubstituted C₁₋₆-alkyl group.

6. The compound according to claim 5 or a pharmaceutically acceptable salt thereof wherein R⁵ is a hydrogen atom.

7. The compound according to claim 1 or a pharmaceutically acceptable salt thereof wherein R² is selected from the group consisting of a hydrogen atom, a methyl group, a halogen atom and a substituted phenyl group.

8. The compound according to claim 7 or a pharmaceutically acceptable salt thereof wherein R² is a hydrogen atom.

9. The compound according to claim 1 or a pharmaceutically acceptable salt thereof wherein R¹ is selected from the group consisting of a hydrogen group and a substituted or unsubstituted C₁₋₆-alkyl group.

10. The compound according to claim 1 or a pharmaceutically acceptable salt thereof wherein R³ is selected from the group consisting of a hydrogen atom, a hydroxy group, a substituted or unsubstituted C₁₋₆-alkyl group, a substituted or unsubstituted C₁₋₆-alkoxy group, a substituted or unsubstituted phenyloxy group and a substituted or unsubstituted phenyl group; and
R⁴ is selected from the group consisting of a hydrogen atom, a fluorine atom, a hydroxy group, a substituted or unsubstituted C₁₋₆-alkyl group and a substituted or unsubstituted C₁₋₆-alkoxy group.

11. The compound according to claim 10 or a pharmaceutically acceptable salt thereof wherein R³ is selected from the group consisting of a hydrogen atom, a hydroxy group, a substituted or unsubstituted C₁₋₆-alkyl group, a substituted or unsubstituted C₁₋₆-alkoxy group and a substituted or unsubstituted phenyl group.

12. The compound according to claim 1 or a pharmaceutically acceptable salt thereof wherein, in the definitions for each substitutent, the substituted or unsubstituted phenyl group is selected from the group consisting of a unsubstituted phenyl group and a hydroxy phenyl group, or
the substituted or unsubstituted phenyloxy group is selected from the group consisting of a unsubstituted phenyloxy group and a hydroxyphenyloxy group.

13. The compound according to claim 12 or a pharmaceutically acceptable salt thereof wherein, in the definitions for each substitutent, the substituted or unsubstituted phenyl group is selected from the group consisting of a unsubstituted phenyl group and a hydroxyphenyl group.

14. A compound of the following formula (II) or a pharmaceutically acceptable salt thereof: 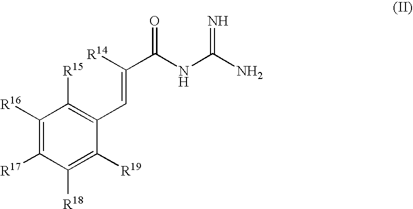
wherein
R¹⁴ is selected from the group consisting of a hydrogen atom, a halogen atom and a substituted or unsubstituted C₁₋₆-alkyl group;
R¹⁵ and R¹⁷ are each independently selected from the group consisting of a hydrogen atom, a halogen atom, a hydroxy group, a substituted or unsubstituted C₁₋₆-alkyl group, a substituted or unsubstituted C₁₋₆-alkoxy group, a substituted or unsubstituted phenyloxy group, a substituted or unsubstituted phenyl group and a substituted or unsubstituted, 5-membered or 6-membered heterocyclic ring having one or more hetero atom(s) selected from the group consisting of a nitrogen atom, an oxygen atom and a sulfur atom, the heterocyclic ring(s) being selected from the group consisting of a pyrrole ring, a furan ring, a thiophene ring, a thiazole ring, an isothiazole ring, an oxazole ring, an isoxazole ring, an imidazole ring, a pyrazole ring, a triazole ring, a tetrazole ring, a pyrimidine ring, a piperazine ring and a morpholine ring, provided that at least one of R¹⁵ and R¹⁷ is a heterocyclic ring; and
R¹⁶, R¹⁸ and R¹⁹ are each independently selected from the group consisting of a hydrogen atom, a halogen atom, a hydroxy group, a substituted or unsubstituted C₁₋₆-alkyl group, a substituted or unsubstituted C₁₋₆-alkoxy group, a substituted or unsubstituted phenyloxy group and a substituted or unsubstituted phenyl group.

15. A compound of the following formula (III) or a pharmaceutically acceptable salt thereof: 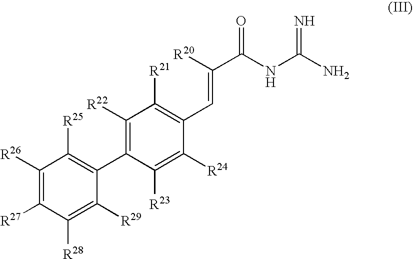
wherein
R²⁰ is selected from the group consisting of a hydrogen atom, a halogen atom and a substituted or unsubstituted C₁₋₆-alkyl group;
R²¹, R²², R²³ and R²⁴ are each independently selected from the group consisting of a hydrogen atom, a halogen atom, a hydroxy group, a substituted or unsubstituted C₁₋₆-alkyl group, a substituted or unsubstituted C₁₋₆-alkoxy group, a substituted or unsubstituted morpholine group and a substituted or unsubstituted piperazine group; and
R²⁵, R²⁶, R²⁷, R²⁸ and R²⁹ are each independently selected from the group consisting of a hydrogen atom, a halogen atom, a nitro group, a nitrile group, a carboxyl group, a hydroxy group, —B(OH)₂, a substituted or unsubstituted amidino group, a substituted or unsubstituted C₁₋₆-alkyl group, a substituted or unsubstituted C₁₋₆-alkenyl group, a substituted or unsubstituted C₁₋₆-alkynyl group, a substituted or unsubstituted C₁₋₆-alkoxy group, a substituted or unsubstituted aminocarbonyl group, a substituted or unsubstituted C₁₋₆-alkylcarbonyl group, a substituted or unsubstituted C₁₋₆-alkoxycarbonyl group and a substituted or unsubstituted C₁₋₆-alkyl-S(═O)₂—NH group, or
two adjacent groups from R²⁶, R²⁷, R²⁸ and R²⁹ together may form a 5-membered or 6-membered heterocyclic ring which has one or two oxygen atom(s) as a hetero atom(s) constituting the ring.

16. The compound according to claim 15 or a pharmaceutically acceptable salt thereof wherein R²² and R²³ are a hydrogen atom.

17. The compound according to claim 15 or a pharmaceutically acceptable salt thereof wherein R²⁰ is selected from the group consisting of a hydrogen atom and a substituted or unsubstituted C₁₋₆-alkyl group.

18. A pharmaceutical composition comprising a compound according to claim 1 or a pharmaceutically acceptable salt thereof and optionally a pharmaceutically acceptable carrier.

19. A pharmaceutical composition for treating or preventing a disease or condition of an organ in which NHE3 is expressed, which comprises a compound according to claim 1 or a pharmaceutically acceptable salt thereof and optionally a pharmaceutically acceptable carrier.

20. A NHE3 inhibitor comprising a compound according to claim 1 or a pharmaceutically acceptable salt thereof and optionally a pharmaceutically acceptable carrier.
